(19) United States
(12) Patent Application Publication
(10) Pub. No.: US 2008/0138800 A1
(43) Pub. Date: Jun. 12, 2008

(54) Multiplexed analysis of polymorphic loci by concurrent interrogation and enzyme-mediated detection

(76) Inventors: Alice Xiang Li, Ithaca, NY (US); Ghazala Hashmi, Lolmdel, NJ (US); Michael Seul, Fanwood, NJ (US)

Correspondence Address: ERIC P. MIRABEZ, BIOARRY SOLUTIONS LLC, 35 TECHNOLOGY DRIVE, WARREN, NJ, 07059, US

(21) Appl. No.: 11/438,740

(22) Filed: May 22, 2006

Related U.S. Application Data
(62) Division of application No. 10/271,602, filed on Oct. 15, 2002.
(60) Provisional application No. 60/329,427, filed on Oct. 15, 2001.
(60) Provisional application No. 60/329,428, filed on Oct. 15, 2001.
(60) Provisional application No. 60/329,619, filed on Oct. 15, 2001.
(60) Provisional application No. 60/329,620, filed on Oct. 15, 2001.

(51) Int. Cl. C12Q 1/68

(52) U.S. Cl. 435/6

(57) ABSTRACT

The invention provides methods and processes for the identification of polymorphisms at one or more designated sites, without interference from non-designated sites located within proximity of such designated sites. Probes are provided capable of interrogation of such designated sites in order to determine the composition of each such designated site. By the methods of this invention, one or more mutations within the CFTR gene and the HLA gene complex can be can be identified.

### CROSS-REFERENCE TO RELATED APPLICATIONS

This application is a divisional application and claims priority to U.S. application Ser. No. 10/271,602, filed Oct. 15, 2002, which claims priority to from U.S. Provisional Application Ser. No. 60/329,427 filed Oct. 14, 2001, U.S. Provisional Application Ser. No. 60/329,620, filed Oct. 15, 2001, U.S. Provisional Application Ser. No. 60/329,428, filed Oct. 14, 2001 and U.S. Provisional Application Ser. No. 60/329,619 filed Oct. 15, 2001. This application is related to PCT application Serial Number PCT/US02/xxxx of the same title filed concurrently herewith. All the above-referenced applications are expressly incorporated herein by reference.

### FIELD OF THE INVENTION

The present invention generally relates to molecular diagnostics and genetic typing or profiling. The invention relates to methods, processes and probes for the multiplexed analysis of highly polymorphic genes. The invention also relates to the molecular typing and profiling of the Human Leukocyte Antigen (HLA) gene complex and the Cystic Fibrosis Conductance Trans-membrane Regulator gene (CFTR) and to compositions, methods and designs relating thereto.

### BACKGROUND OF THE INVENTION

The ability to efficiently, rapidly and unambiguously analyze polymorphisms in the nucleic acid sequences of a gene of interest plays an important role in the development of molecular diagnostic assays, the applications of which includes genetic testing, carrier screening, genotyping or genetic profiling, and identity testing. For example, it is the objective of genetic testing and carrier screening to determine whether mutations associated with a particular disease are present in a gene of interest. The analysis of polymorphic loci, whether or not these comprise mutations known to cause disease, generally provides clinical benefit, as for example in the context of pharmacogenomic genotyping or in the context of HLA molecular typing, in which the degree of allele matching in the HLA loci of transplant donor and prospective recipient is determined in context of allogeneic tissue and bone marrow transplantation.

The multiplexed analysis of polymorphisms, while desirable in facilitating the analysis of a high volume of patient samples, faces a considerable level of complexity which will likely increase as new polymorphisms, genetic markers and mutations are identified and must be included in the analysis. The limitations of current methods to handle this complexity in a multiplexed format of analysis so as to ensure reliable assay performance while accommodating high sample volume, and the consequent need for novel methods of multiplexed analysis of polymorphisms and mutations is the subject of the present invention. By way of example, the genetic loci encoding Cystic Fibrosis Transmembrane Conductance (CFTR) channel and Human Leukocyte Antigens (HLA) are analyzed by the methods of the invention. Cystic fibrosis (CF) is one of the most common recessive disorders in Caucasians with a rate of occurrence in the US of 1 in 2000 live births. About 4% of the population carry one of the CF mutations. The CFTR gene is highly variable: more than 900 mutations have been identified to date (see the Cystic Fibrosis Mutation Database, maintained by the laboratory of Lap-Chee Tsui (available online) http://www.genet.sickkids.on.ca/cftr, which is incorporated herein by reference). The characterization of the CFTR gene provides the key to the molecular diagnosis of CF by facilitating the development of sequence-specific probes (Rommens et al., 1989; Riordan, et al., 1989; Kerem et al., 1989, each of which is incorporated herein by reference). The National Institutes of Health (NIH)-sponsored consensus development conference recommended carrier screening for CFTR mutations for adults with a positive family history of CF (NIH 1997). The committee on carrier screening of the American College of Medical Genetics (ACMG) has recommended for use in general population carrier screening a pan-ethnic mutation panel that includes a set of 25 disease-causing CF mutations with an allele frequency of >0.1% in the general population of United States (see the American College of Medical Genetics website http://www.faseb.org/genetics/acmg, which is incorporated herein by reference). The mutations in the ACMG panel also include the most common mutations in Ashkenazi Jewish and African-American populations.

Several methods have been described for the detection of CFTR mutations including the following: [[:]]denaturing gradient gel electrophoresis (Devoto et al., 1991); single strand conformation polymorphism analysis (Plieth et al., 1992); RFLP (Friedman et al., 1991); amplification with allele-specific primers (ASPS) (Gremonesi et al., 1992), and probing with allele specific oligonucleotides (ASO) (Saiki et al., 1986). A widely used method involves PCR amplification followed by blotting of amplified target strands onto a membrane and probing of strands with oligonucleotides designed to match either the normal (“wild type”) or mutant configuration. Specifically, multiplex PCR has been used in conjunction with ASO hybridization in this dot blot format to screen 12 CF mutations (Shuber et al., 1993). In several instances, arrays of substrate-immobilized oligonucleotide probes were used to facilitate the detection of known genomic DNA sequence variations (Saiki, R K et al., 1989) in a “reverse dot blot” format An array of short oligonucleotides synthesized in-situ by photolithographic processes was used to detect known mutations in the coding region of the CFTR gene (Cronin, M T., et al., 1996). Primer extension using reverse transcriptase has been reported as a method for detecting the Δ508 mutation in CFTR (Pastinen, T., 2000). This approach was described as early as 1989 (Wu, D. Y. et al, Proc. Natl. Acad. Sci. USA. 86:2757-2760 (1989), Newton, C. R. et al., Nucleic Acids Res. 17:2503-2506 (1989)). As further discussed herein below, while providing reasonable detection in a research laboratory setting, these methods require significant labor, provide only slow turnaround, offer only low sample throughput, and hence require a high cost per sample.

In connection with the spotted microarrays, several methods of spotting have been described, along with many substrate materials and methods of probe immobilization. However, the spotted arrays of current methods exhibit not only significant array-to-array variability but also significant spot-to-spot variability, an aspect that leads to limitations in assay reliability and sensitivity. In addition, spotted arrays are difficult to miniaturize beyond their current spot dimensions of typically 100 μm diameter on 500 μm centers, thereby increasing total sample volumes and contributing to slow assay kinetics limiting the performance of hybridization assays whose completion on spotted arrays may require as much as 18 hours. Further, use of spotted arrays involves readout via highly specialized confocal laser scanning apparatus. In an alternative approach, oligonucleotide arrays synthesized in-situ by a photolithographic process have been described. The complexity of array fabrication, however, limits routine customization and combines considerable expense with lack of flexibility for diagnostic applications.

The major histocompatibility complex (MHC) includes the human leukocyte antigen (HLA) gene complex, located on the short arm of human chromosome six. This region encodes cell-surface proteins which regulate the cell-cell interactions underlying immune response. The various HLA Class I loci encode 44,000-dalton polypeptides which associate with β-2 microglobulin at the cell surface and mediate the recognition of target cells by cytotoxic T lymphocytes. HLA Class II loci encode cell surface heterodimers, composed of a 29,000-dalton and a 34,000-dalton polypeptide which mediate the recognition of target cells by helper T lymphocytes. HLA antigens, by presenting foreign pathogenic peptides to T-cells in the context of a “self” protein, mediate the initiation of an immune response. Consequently, a large repertoire of peptides is desirable because it increases the immune response potential of the host. On the other hand, the correspondingly high degree of immunogenetic polymorphism represents significant difficulties in allotransplantation, with a mismatch in HLA loci representing one of the main causes of allograft rejection. The degree of allele matching in the HLA loci of a donor and prospective recipient is a major factor in the success of allogeneic tissue and bone marrow transplantation.

The HLA-A, HLA-B, and HLA-C loci of the HLA Class I region as well as the HLA-DRB, HLA-DQB, HLA-DQA, HLA-DPB and HLA-DPA loci of the HLA Class II region exhibit an extremely high degree of polymorphism. To date, the WHO nomenclature committee for factors of the HLA system has designated 225 alleles of HLA A (HLA A*0101, A*0201, etc.), 444 alleles of HLA-B, and 111 alleles of HLA-C, 358 HLA-DRB alleles, 22 HLA-DQA alleles, 47 HLA-DQB alleles, 20 HLA-DPA alleles and 96 HLA-DPB alleles (See see IMGT/HLA Sequence Database, European Bioinformatics Institute (available online) http://www3.ebi.ac.uk:80/imgt/hla/indcx.html) and Schreuder, G. M. Th. et al, Tissue Antigens,[[.]] 54:409-437 (1999)[[)]], both of which are hereby incorporated by reference.)

HLA typing is a routine procedure that is used to determine the immunogenetic profile of transplant donors. The objective of HLA typing is the determination of the patient's allele configuration at the requisite level of resolution, based on the analysis of a set of designated polymorphisms within the genetic locus of interest. Increasingly, molecular typing of HLA is the method of choice over traditional serological typing, because it eliminates the requirement for viable cells, offers higher allelic resolution, and extends HLA typing to Class II for which serology has not been adequate (Erlich, H. A. et al, Immunity. 14:347-356 (2001)).

One method currently applied to clinical HLA typing uses the polymerase chain reaction (PCR) in conjunction with sequence-specific oligonucleotide probes (SSO or SSOP), which are allowed to hybridize to amplified target sequences to produce a pattern as a basis for HLA typing.

The availability of sequence information for all available HLA alleles has permitted the design of sequence-specific oligonucleotides (SSO) and allele-specific oligonucleotides (ASO) for the characterization of known HLA polymorphisms as well as for sequencing by hybridization (Saiki, R. K. Nature 324:163-166 (1986), Cao, K. et al, Rev Immunogenetics, 1999:1:177-208).

In one embodiment of SSO analysis, also referred to as a “dot blot format”, DNA samples are extracted from patients, amplified and blotted onto a set of nylon membranes in an 8×12 grid format. One radio-labeled oligonucleotide probe is added to each spot on each such membrane; following hybridization, spots are inspected by autoradiography and scored either positive (1) or negative (O). For each patient sample, the string of 1's and 0's constructed from the analysis of all membranes defines the allele configuration. A multiplexed format of SSO analysis in the “reverse dot blot format” employs sets of oligonucleotide probes immobilized on planar supports (Saiki, R. et al, Immunological Rev. 167: 193-199 (1989), Erlich, H. A. Eur. J. Immunogenet. 18: 33-55 (1991)).

Another method of HLA typing uses the polymerase-catalyzed elongation of sequence-specific primers (SSPs) to discriminate between alleles. The high specificity of DNA polymerase generally endows this method with superior specificity. In the SSP method, PCR amplification is performed with a specific primer pair for each polymorphic sequence motif or pair of motifs and a DNA polymerase lacking 3′->5′ exonuclease activity so that elongation (and hence amplification) occurs only for that primer whose 3′ terminus is perfectly complementary (“matched”) to the template. The presence of the corresponding PCR product is ascertained by gel electrophoretic analysis. An example of a highly polymorphic locus is the 280 nt DNA fragment of the HLA class II DR gene which features a high incidence of polymorphisms

HLA typing based on the use of sequence-specific probes (SSP), also referred to as phototyping (Dupont, B. Tissue Antigen. 46: 353-354 (1995)), has been developed as a commercial technology that is in routine use for class I and class II typing (Bunce, M. et al, Tissue Antigens. 46:355-367 (1995), Krausa, P. and Browning, M. J., Tissue Antigens. 47: 237-244 (1996), Bunce, M. et al, Tissue Antigens. 45:81-90 (1995)). However, the requirement of the SSP methods of the prior art for extensive gel electrophoretic analysis for individual detection of amplicons represents a significant impediment to the implementation of multiplexed assay formats that can achieve high throughput. This disadvantage is overcome by the methods of the present invention.

In the context of elongation reactions, highly polymorphic loci and the effect of non-designated polymorphic sites as interfering polymorphisms were not considered in previous applications, especially in multiplexed format. Thus, there is a need to provide for methods, compositions and processes for the multiplexed analysis of polymorphic loci that would enable the detection of designated while accommodating the presence of no-designated sites and without interference from such non-designated sites.

### SUMMARY OF THE INVENTION

The present invention provides methods and processes for the concurrent interrogation of multiple designated polymorphic sites in the presence of non-designated polymorphic sites and without interference from such non-designated sites. Sets of probes are provided which facilitate such concurrent interrogation. The present invention also provides methods, processes, and probes for the identification of polymorphisms of the HLA gene complex and the CFTR gene.

The specificity of methods of detection using probe extension or elongation is intrinsically superior to that of methods using hybridization, particularly in a multiplexed format, because the discrimination of sequence configurations no longer depends on differential hybridization but on the fidelity of enzymatic recognition. To date, the overwhelming majority of applications of enzyme-mediated analysis use single base probe extension. However, probe elongation, in analogy to that used in the SSP method of HLA typing, offers several advantages for the multiplexed analysis of polymorphisms, as disclosed herein. Thus, single-nucleotide as well as multi-nucleotide polymorphisms are readily accommodated. The method, as described herein, is generally practiced with only single label detection, accommodates concurrent as well as consecutive interrogation of polymorphic loci and incorporates complexity in the probe design.

One aspect of this invention provides a method of concurrent determination of nucleotide composition at designated polymorphic sites located within one or more target nucleotide sequences. This method comprises the following steps: (a) providing one or more sets of probes, each probe capable of annealing to a subsequence of the one or more target nucleotide sequences located within a range of proximity to a designated polymorphic site; (b) contacting the set of probes with the one or more target nucleotide sequences so as to permit formation of hybridization complexes by placing an interrogation site within a probe sequence in direct alignment with the designated polymorphic site; (c) for each hybridization complex, determining the presence of a match or a mismatch between the interrogation site and a designated polymorphic site; and (d) determining the composition of the designated polymorphic site.

Another aspect of this invention is to provide a method of sequence-specific amplification of assay signals produced in the analysis of a nucleic acid sequence of interest in a biological sample. This method comprises the following steps: (a) providing a set of immobilized probes capable of forming a hybridization complex with the sequence of interest; (b) contacting said set of immobilized probes with the biological sample containing the sequence of interest under conditions which permit the sequence of interest to anneal to at least one of the immobilized probes to form a hybridization complex; (c) contacting the hybridization complex with a polymerase to allow elongation or extension of the probes contained within the hybridization complex; (d) converting elongation or extension of the probes into an optical-signal; and (e) recording the optical signal from the set of immobilized probes in real time.

Yet another aspect of this invention is to provide a method of forming a covering probe set for the concurrent interrogation of a designated polymorphic site located in one or more target nucleic acid sequences. This method comprises the steps of: (a) determining the sequence of an elongation probe capable of alignment of the interrogation site of the probe with a designated polymorphic site; (b) further determining a complete set of degenerate probes to accommodate all non-designated as well as non-selected designated polymorphic sites while maintaining alignment of the interrogation site of the probe with the designated polymorphic site; and (c) reducing the degree of degeneracy by removing all tolerated polymorphisms.

One aspect of this invention is to provide a method for identifying polymorphisms at one or more designated sites within a target polynucleotide sequence. This the method comprise the following steps: (a) providing one or more probes capable of interrogating said designated sites; (b) assigning a value to each such designated site while accommodating non-designated polymorphic sites located within a range of proximity to each such polymorphism.

Another aspect of this invention is to provide a method for determining a polymorphism at one or more designated sites in a target polynucleotide sequence. This method comprises providing a probe set for the designated sites and grouping the probe set in different probe subsets according to the terminal elongation initiation of each probe.

Another aspect of this invention is to provide a method for the concurrent interrogation of a multiplicity of polymorphic sites comprising the step of conducting a multiplexed elongation assay by applying one or more temperature cycles to achieve linear amplification of such target.

Yet another aspect of this invention is to provide a method for the concurrent interrogation of a multiplicity of polymorphic sites. This method comprises the step of conducting a multiplexed elongation assay by applying a combination of annealing and elongation steps under temperature-controlled conditions.

Another aspect of this invention is to provide a method of concurrent interrogation of nucleotide composition at S polymorphic sites, P_{S}:={c_{P}(s); 1≦s≦S} located within one or more contiguous target sequences, said method assigning to each cₚ one of a limited set of possible values by performing the following steps: (a) providing a set of designated immobilized oligonucleotide probes, also known as elongation probes, each probe capable of annealing in a preferred alignment to a subsequence of the target located proximal to a designated polymorphic site, the preferred alignment placing an interrogation site within the probe sequence in direct juxtaposition to the designated polymorphic site, the probes further containing a terminal elongation initiation (TEI) region capable of initiating an elongation or extension reaction; (b) permitting the one or more target sequences to anneal to the set of immobilized oligonucleotide probes so as form probe-target hybridization complexes; and (c) for each probe-target hybridization complex, calling a match or a mismatch in composition between interrogation site and corresponding designated polymorphic site.

Other objects, features and advantages of the invention will be more clearly understood when taken together with the following detailed description of an embodiment, which will be understood as being illustrative only.

### BRIEF DESCRIPTION OF THE DRAWINGS

FIG. 1a is an illustration of probe sets designed to interrogate designated sites in HLA-DR and an internal control.

FIG. 1b is an illustration of a staggered primer design.

FIG. 2 is an illustration of a modification of allele binding pattern based on tolerance effect.

FIG. 3 is an illustration of the use of linked primer structure to separate the anchoring sequence and polymorphism detection sequence.

FIG. 4 shows simulated ambiguity in allele identification due to allele combination.

FIG. 5 shows one method for decreasing the ambiguity in allele identification that arises from allele combination.

FIG. 6 is an illustration of a combination of hybridization and elongation.

FIG. 7 shows a model reaction using synthetic oligonucleotides as targets.

FIG. 8 shows results obtained using testing real patient sample in an eMAP format.

FIG. 9 shows results obtained from eMAP primer extension for DR locus.

FIG. 10 shows results obtained from eMAP for DR locus.

FIG. 11 shows results obtained from eMAP for A locus Exon 3.

FIG. 12 shows results obtained from eMAP SSP for A locus Exon 3 and is an example of tolerance for the non-designated polymorphism.

FIG. 13 is an illustration of bead immobilized probe elongation of variable mutant sites.

FIG. 14 is an illustration of PCR using primers immobilized on the surface of beads.

FIG. 15 is an illustration of elongation of multiple probes using combined PCR products.

FIG. 16 is an illustration of results for probe elongation of a multiplexed CF mutation.

FIG. 16a is an illustration of probe elongation using a synthetic target. FIG. 16b is an illustration of probe elongation using beads in a PCR reaction.

FIG. 17 is an illustration of one-step elongation with temperature-controlled cycling results.

FIG. 18 is an illustration of primer elongation with labeled dNTP and three other unlabeled dNTPs.

FIG. 19 is an illustration of primer elongation with labeled ddNTP and three other unlabeled dNTPs.

FIG. 20 is an illustration of primer elongation, where four unlabeled dNTPs are used for elongation and the product is detected by a labeled oligonucleotide probe, which hybridizes to the extended unlabeled product.

FIG. 21 is an illustration of a primer extension in which a labeled target and four unlabeled dNTPs are added. This illustration which shows that only with the extended product can the labeled target be retained with the beads when high temperature is applied to the chip.

FIG. 22 is an illustration of linear amplification where sequence specific probes are immobilized.

FIG. 23 is an illustration of the utilization of hairpin probes.

FIG. 24 is an illustration of applying this invention to the analysis of cystic fibrosis and Ashkenazi Jewish disease mutations.

### DETAILED DESCRIPTION OF THE INVENTION

This invention provides compositions, methods and designs for the multiplexed analysis of highly polymorphic loci; that is, loci featuring a high density of specific (“designated”) polymorphic sites, as well as interfering non-designated polymorphic sites. The multiplexed analysis of such sites thus generally involves significant overlap in the sequences of probes directed to adjacent sites on the same target, such that probes designed for any specific or designated site generally also will cover neighboring polymorphic sites. The interference in the analysis of important genes including CFTR and HLA has not been addressed in the prior art. To exemplify the methods of the methods of the invention, the HLA gene complex and the CFTR gene are analyzed.

The present invention provides compositions and methods for the parallel or multiplexed analysis of polymorphisms (“MAP”) in nucleic acid sequences displaying a high density of polymorphic sites. In a given nucleic acid sequence, each polymorphic site comprises a difference comprising one or more nucleotides.

This invention provides methods and compositions for the concurrent interrogation of an entire set of designated polymorphisms within a nucleic acid sequence. This invention provides compositions, methods and designs to determine the composition at each such site and thereby provide the requisite information to select, from the set of possible configurations for the sequence of interest, the actual configuration in a given specific sample. The invention also serves to narrow the set of possible sequences in that sample. Accordingly, in certain embodiments, it will be useful or necessary to determine sequence composition by assigning to a designated site one of the possible values corresponding to nucleotide identity. In other embodiments, it will be sufficient to determine the site composition to be either matching or non-matching with respect to a known reference sequence, as in the assignment of “wild-type” or “mutation” in the context mutation analysis. The capability of sequence determination thereby afforded is referred to herein as confirmatory sequencing or resequencing. In a preferred embodiment, the present invention provides elongation-mediated multiplexed analysis of polymorphisms (eMAP) of the Cystic Fibrosis Transmembrane Conductance Regulator (CFTR) gene and for the Human Leukocyte Antigen (HLA) gene complex.

The methods and compositions of this invention are useful for improving the reliability and accuracy of polymorphism analysis of target regions which contain polymorphic sites in addition to the polymorphic sites designated for interrogation. These non-designated sites represent a source of interference in the analysis. Depending on the specific assay applications, one or more probes of differing composition may be designated for the same polymorphic site, as elaborated in several Examples provided herein. It is a specific objective of the present invention to provide compositions and methods for efficient, rapid and unambiguous analysis of polymorphisms in genes of interest. This analysis is useful in molecular diagnostic assays, such as those designed, for example, for genetic testing, carrier screening, genotyping or genetic profiling, identity testing, paternity testing and forensics.

Preparation of target sequences may be carried out using methods known in the art. In a non-limiting example, a sample of cells or tissue is obtained from a patient. The nucleic acid regions containing target sequences (e.g., Exons 2 and 3 of HLA) are then amplified using standard techniques such as PCR (e.g., asymmetric PCR).

Probes for detecting polymorphic sites function as the point of initiation of a polymerase-catalyzed elongation reaction when the composition of a polymorphic site being analyzed is complementary (“matched”) to that of the aligned site in the probe. Generally, the probes of the invention should be sufficiently long to avoid annealing to unrelated DNA target sequences. In certain embodiments, the length of the probe may be about 10 to 50 bases, more preferably about 15 to 25, and more preferably 18 to 20 bases. Probes may be immobilized on the solid supports via linker moieties using methods and compositions well known in the art.

As used herein, the term “nucleic acid” or “oligonucleotide” refers to deoxyribonucleic acid or ribonucleic acid in a single or double-stranded form. The term also covers nucleic-acid-like structures with synthetic backbones. DNA backbone analogues include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs). See Oligonucleotides and Analogues, A Practical Approach (Editor: F. Eckstein), IRL Press at Oxford University Press (1991); Antisense Strategies, Annals of the New York Academy of Sciences, vol. 600, Eds.; Baserga and Denhardt (NYAS 1992); Milligan, J. Med. Chem., vol. 36, pp. 1923-1937; Antisense Research and Applications (1993, CRC Press). PNAs contain non-ionic backbones, such as N-2(2-aminoethyl) glycine units. Phosphorothioate linkages are described in WO 97/03211; WO 96/39159; and Mata, Toxicol. Appl. Pharmacol. 144: 189-197 (1997). Other synthetic backbones encompassed by the term include methyl-phosphonate linkages or alternating methylphosphonate and phosphodiester linkages (Strauss-Soukup, Biochemistry, 36: 8692-8698 (1997), and benzylphosphonate linkages (Samstag, Antisense Nucleic Acid Drug Dev., 6: 153-156 (1996)). The term nucleic acid includes genes, cDNAs, and mRNAs.

As used herein, the term “hybridization” refers to the binding, duplexing, or hybridizing of a nucleic acid molecule preferentially to a particular nucleotide sequence under stringent conditions. The term “stringent conditions” refers to conditions under which a probe will hybridize preferentially to the corresponding target sequence, and to a lesser extent or not at all to other sequences. A “stringent hybridization” is sequence dependent, and is different under different conditions. An extensive guide to the hybridization of nucleic acids may be found in, e.g. Tijssen, Laboratory Techniques in Biochemistry and Molecular Biology, Elsevier, NY (1993). Generally, highly stringent hybridization and wash conditions are selected to about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected by conducting the assay at a temperature set to be equal to the Tₘ for a particular probe. An example of highly stringent wash condition is 0.15 M NaCl at 72° C. for about 15 minutes. An example of stringent wash conditions is a 0.2× SSC wash at 65° C. for 15 minutes. See Sambrook, Molecular Cloning: A Laboratory Manual (2^{nd} Ed), vol. 1-3 (1989).

As used herein, the term “designated site” is defined as a polymorphic site of interest (i.e., a polymorphic site that one intends to identify) on a given nucleic acid. The term “non-designated site” refers to any polymorphic site that co-exists with a designated site or sites on a given nucleic acid but is not of interest.

As used herein, the term “correlated designated sites” refers to polymorphic sites with correlated occurrences. Typically, each member of such a set of polymorphic sites must be identified in order to identify the allele to which the set belongs.

As used herein, the term “selected designated site” refers to a polymorphic site of interest on a given nucleic acid that also overlaps with the 3′ end of a probe sequence of this invention. A “non-selected designated site” refers to a polymorphic site of interest that does not overlap with a 3′ end of a probe sequence of this invention.

As used herein, an “interfering non-designated site” refers to a non-designated polymorphic site that is within 1-5 bases from the 3′ end of a probe sequence of this invention. A “non-interfering non-designated site” refers to a non-designated site that is greater than 5 bases from the 3′ end of a probe sequence of this invention. The non-interfering non-designated site may be closer to the 5′ end of the probe sequence than to the 3′ end.

In certain embodiments, the probes of this invention comprise a “terminal elongation initiation” region (also referred to as a “TEI” region) and a Duplex Anchoring (“DA”) region. The TEI region refers a section of the probe sequence, typically the three or four 3′ terminal positions of the probe. The TEI region is designed to align with a portion of the target nucleic acid sequence at a designated polymorphic site so as to initiate the polymerase-catalyzed elongation of the probe. The DA region[[,]] typically comprises the remaining positions within the probe sequence and is preferably designed to align with a portion of the target sequence in a region located close (within 3-5 bases) to the designated polymorphism.

As used herein, the term a “close range of proximity” refers to a distance of between 1-5 bases along a given nucleic acid strand. A “range of proximity” refers to a distance within 1-10 bases along a given nucleic acid strand. The term “range of tolerance” refers to the total number of mismatches in the TEI region of a probe hybridized to a target sequence that still permits annealing and elongation of the probe. Typically, more than 2 mismatches in the TEI region of a hybridized probe is beyond the range of tolerance.

The terms “microspheres”, “microparticles”, “beads”, and “particles” are herein used interchangeably. The composition of the beads includes, but is not limited to, plastics, ceramics, glass, polystyrene, methylstyrene, acrylic polymers, paramagnetic materials, thoria sol, carbon graphite, titanium dioxide, latex or cross-linked dextrans such as sepharose, cellulose, nylon, cross-linked micelles and Teflon. See “Microsphere Detection Guide” from Bangs Laboratories, Fishers IN. The particles need not be spherical and may be porous. The bead sizes may range from nanometers (e.g., 100 nm) to millimeters (e.g., 1 mm), with beads from about 0.2 micron to about 200 microns being preferred, more preferably from about 0.5 to about 5 micron being particularly preferred.

This invention provides for the concurrent interrogation of a set of designated polymorphic sites within one or more target strands by first annealing a set of immobilized sequence specific oligonucleotide probes to target nucleic acid strands and by probing the configuration of designated polymorphic sites by way of polymerase-catalyzed elongation of the annealed set of immobilized sequence-specific oligonucleotide probes. An elongation probe is designed to interrogate a designated site by annealing to a sequence in a given target, thereby forming a hybridization complex (“duplex”). The probe's 3′ terminus is placed at or near the designated site within the target and polymerase-catalyzed probe elongation is initiated if the 3′ terminal probe composition matches (i.e., is complementary to) that of the target at the interrogation site. As described herein, the probe may be designed to anneal in a manner such that the designated site is within a range of proximity of the 3′ terminus.

In one embodiment of the invention, two or more probes may be provided for interrogation of a specific designated site. The probes are designed to take into account the possibility of polymorphisms or mutations at the interrogation site and non-designated polymorphic sites within a certain range of proximity of the designated polymorphic site. In this context, the term “polymorphism” refers to any variation in a nucleic acid sequence, while the term “mutation” refers to a sequence variation in a gene that is associated or believed to be associated with a phenotype. In a preferred embodiment, this multiplicity of probe sequences contains at least one probe that matches the specific target sequence in all positions within the range of proximity to ensure elongation.

In certain embodiments, the invention discloses compositions and methods for the parallel interrogation of S polymorphic sites selected from a target sequence of length N by a set of L≧S oligonucleotide primers.

In accordance with the requirements of specific assay applications, one or more probes of differing composition may be designated for the same polymorphic site, as elaborated in several Examples provided herein.

Each designated probe is composed of a nucleotide sequence of length M which contains an interrogation site (one that, upon hybridization, aligns with the polymorphic site being analyzed) at or near the 3′ terminus. Although 3′ end is preferred, those within 3-4 bases from the 3′ end may be used. The primer is immobilized on a solid phase carrier (may be linked via a linker sequence or other linker moiety) and is identified by its association with that carrier. The probe sequence is designed to permit annealing of the primer with the target so as to form a hybridization complex between probe and target and to ensure the alignment of the interrogation site with the designated polymorphic site, the preferred configuration providing an interrogation site at the probe's 3′ terminus and alignment of the 3′ terminus with the designated polymorphic site. The step of interrogating the nucleotide composition of the designated polymorphic site with a designated probe of given interrogation site composition assigns to that site one of two values, namely matched, numerically represented by 1, or non-matched, numerically represented by 0. In HLA molecular typing, the resulting binary string of length L identifies an allele to a desired typing resolution.

In a preferred embodiment, the interrogation step uses the extension of the designated probe. This reaction, catalyzed by a polymerase, produces an extended hybridization complex by adding to the probe sequence one or more nucleoside triphosphates in the order reflecting the sequence of the target sequence in the existing hybridization complex. In order for this extension reaction to proceed, a designated primer of length M must contain a terminal extension initiation region of length M*≦M, herein also referred to as terminal extension initiation sequence (or TEI sequence), which contains the interrogation site. Extension proceeds if the composition of the designated interrogation site matches that of the designated polymorphic site.

Methods of the prior art of detecting successful extension have been described which involve the use labeled deoxy nucleoside triphosphates (dNTPs) or dideoxy nucleoside triphosphates (ddNTPs). The present invention also discloses novel methods of providing optical signatures for detection of successful extension eliminating the need for labeled dNTPs or ddNTPs, an advantage arising from the reduction in the efficiency of available polymerases in accommodating labeled dNTPs or ddNTPs.

However, the density of polymorphic sites in highly polymorphic loci considered in connection with the present invention makes it likely that designated primers directed to selected polymorphic sites, when annealing to the target subsequence proximal to the designated polymorphic site, will overlap adjacent polymorphic sites.

That is, an oligonucleotide probe, designed to interrogate the configuration of the target at one of the selected polymorphic sites, and constructed with sufficient length to ensure specificity and thermal stability in annealing to the correct target subsequence, will align with other nearby polymorphic sites. These interfering polymorphic sites may include the non-designated sites as well as non-selected designated sites in the target sequence.

In a multiplexed SSP reaction carried out in solution, the partial overlap between designated probes directed to nearby selected polymorphisms may lead to mutual competition between probes for the same target. The present invention significantly reduces this complication by way of probe immobilization.

As with multiplexed differential hybridization generally, the mismatch in one or more positions between a designated probe and target may affect the thermal stability of the hybridization complex. That is, any set of annealing conditions applied to the entire reaction mixture may produce varying degrees of annealing between probe and target and may affect the outcome of the subsequent probe extension reaction, thereby introducing ambiguities in the assay which may require subsequent resequencing.

Non-designated polymorphic sites located in immediate proximity to the interrogation site near or at the 3′ terminus of the designated probe are particularly deleterious to the effectiveness of the probe's TEI sequence in initiating the extension reaction.

The power of currently available polymerase enzymes catalyzing the extension reaction to discriminate between a match and a mismatch in composition between the interrogation site within the designated primer and the polymorphic site depends on the displacement of the interrogation site from the primer's 3′ terminus, considering single nucleotide as well as multiple nucleotide polymorphisms.

In a preferred embodiment yielding optimal discriminating power, the interrogation site is provided at the probe's 3′ terminus. Given a probe sequence of length M designated for a selected site s* in the representation P_{M(s*)}:={c_{P(m)}; 1≦m≦M}, the index m increasing in the primer's 5′ to 3′ direction, this configuration provides for alignment of the designated site s* with position M in the probe sequence; in the case of multiple nucleotide polymorphisms, positions M-1 (for a dinucleotide polymorphism) and M-2 (for a trinucleotide polymorphism), etc. also are implicated.

Under these circumstances as they are anticipated in the multiplexed analysis of highly polymorphic loci, the advantage of enhanced specificity afforded by the application of a polymerase-catalyzed extension reaction is greatly diminished or lost as a result of complications arising from “sub-optimal” annealing conditions closely related to those limiting the performance of SSO analysis.

In connection with the optimization of the design of multiple probe sequences sharing the same interrogation site composition for any given designated polymorphic site, it will be useful to consider the concept of tolerance of interfering polymorphisms. Considering without limitation of generality the example of the single nucleotide polymorphism, a shift in alignment of s* away from the 3′ terminus to positions M-1, M-2, . . . , M-m* leads to a gradually diminished discriminatory power. That is, when the designated polymorphic site is aligned with an interior probe position, m*, the extension reaction no longer discriminates between match and mismatch. Conversely, in the preferred embodiment of placing the interrogation site at the probe's 3′ terminus, the deleterious effect of nearby non-designated polymorphisms on the effectiveness of the extension reaction likewise decreases with distance from the 3′ terminus. That is, non-designated polymorphisms aligned with position between 1 and m* will not affect the extension reaction.

The terminal sequence of length M-m*+1 within the probe is herein referred to as the TEI sequence of a given primer. In general, 1<m*<M, and the TEI sequence may comprise only small number of terminal probe positions; in certain cases, m*=1, so that the probe sequence encompasses the entire probe sequence.

The present invention accommodates the presence of interfering polymorphic sites within the length of a designated probe sequence by taking into account these known sequence variations in the design of multiple probes. In particular, the number of alternate probe sequence configurations to be provided for given probe length M is significantly reduced as a result of the existence of a TEI sequence of length M−m*+1. That is, in order to ensure effective discriminatory power of the extension reaction, it is sufficient to restrict the anticipatory alternate probe sequence configurations to the length of the TEI sequence. In a preferred embodiment, all possible alternative sequences are anticipated so that one of these alternate probe sequences will match the target in all of the positions m*, m*+1, . . . M−1, M.

Providing, for each selected polymorphic site, a multiplicity of designated probes with anticipatory sequences increases the complexity of coding if all of these probes are separately encoded by the unique association with coded solid phase carriers. However, this complexity is reduced by placing this set of probes on a common solid phase carrier. That is, only the interrogation site composition of any designated probes is encoded, a concept herein referred to as TEI sequence pooling or probe pooling. Complete probe sequence pooling reduces the coding complexity to that of the original design in which no anticipatory probe sequences were provided. Partial pooling also is possible.

In certain preferred embodiments, the polymerase used in probe elongation is a DNA polymerase that lacks 3′ to 5′ exonuclease activity. Examples of such polymerases include T7 DNA polymerase, T4 DNA polymerase, ThermoSequenase and Taq polymerase. When the target nucleic acid sequence is RNA, reverse transcriptase may be used. In addition to polymerase, nucleoside triphosphates are added, preferably all four bases. For example dNTPs, or analogues, may be added. In certain other embodiments, ddNTPs may be added. Labeled nucleotide analogues, such as Cye3-dUTP may also be used to facilitate detection.

Prior art methods for detecting successful elongation have been described which use labeled deoxy nucleoside triphosphates (dNTPs) or dideoxy nucleoside triphosphates (ddNTPs). This invention discloses novel methods of providing optical signatures for detecting successful elongation, thus eliminating the need for labeled dNTPs or ddNTPs. This is advantageous because currently available polymerases are less efficient in accommodating labeled dNTPs or ddNTPs.

This invention provides methods and compositions for accurate polymorphism analysis of highly polymorphic target regions. As used herein, highly polymorphic sequences are those containing, within a portion of the sequence contacted by the probe, not only the designated or interrogated polymorphic site, but also non-designated polymorphic sites which represent a potential source of error in the analysis. Analogous considerations pertain to designs, compositions and methods of multiplexing PCR reactions. In a preferred embodiment, covering sets of PCR probes composed of priming and annealing subsequences are displayed on encoded microparticles to produce bead-displayed amplicons by probe elongation. Assemblies of beads may be formed on planar substrates, prior to or subsequent to amplification to facilitate decoding and imaging of probes.

In one embodiment, this invention provides probes that are designed to contain a 3′ terminal “priming” subsequence, also referred to herein as a Terminal Elongation Initiation (TEI) region, and an annealing subsequence, also referred to herein as a Duplex Anchoring (DA) region. The TEI region typically comprises the three or four 3′ terminal positions of a probe sequence. The TEI region is designed to align with a portion of the target sequence at a designated polymorphic site so as to initiate the polymerase-catalyzed elongation of the probe. Probe elongation indicates a perfect match in composition of the entire TEI region and the corresponding portion of the target sequence. The DA region, comprising remaining positions within the probe sequence, is preferably designed to align with a portion of the target sequence in a region located close (within 3-5 bases) to the designated polymorphism. The duplex anchoring region is designed to ensure specific and strong annealing, and is not designed for polymorphism analysis. As described herein, the DA and TEI regions may be located immediately adjacent to one another within the probe or may be linked by a molecular tether. The latter approach permits flexibility in the placement of DA region so as to avoid non-designated polymorphisms located immediately adjacent to the designated site. The composition and length of the DA region are chosen to facilitate the formation of a stable sequence-specific hybridization complex (“duplex”), while accommodating (i.e., taking into account) the presence of one or more non-designated polymorphisms located in that region of the target. The length of the annealing subsequence is chosen to minimize cross-hybridization by minimizing sequence homologies between probe and non-selected subsequences of the target. The length of the annealing subsequence generally exceeds that of the priming subsequence so that failure to form a duplex generally implies failure to produce an elongation product.

The elongation reaction provides high specificity in detecting polymorphisms located within the TEI region. For non-designated polymorphisms in the DA region, the elongation reaction will proceed at a level either comparable to, or lower than that of the perfect match under certain conditions. This is referred to as the tolerance effect of the elongation reaction. Tolerance is utilized in the design of probes to analyze designated and non-designated polymorphisms as described in examples herein.

The density of polymorphic sites in the highly polymorphic loci considered in certain embodiments of this invention makes it likely that probes directed to designated polymorphic sites will overlap adjacent polymorphic sites, when annealing to a target subsequence proximal to the designated polymorphic site. That is, an oligonucleotide probe designed to interrogate the configuration of the target at a selected designated polymorphic site, and constructed with sufficient length to ensure specificity and thermal stability in annealing to the correct target subsequence will align with nearby polymorphic sites. These interfering polymorphic sites may include non-designated sites in the target sequence as well as designated but not selected polymorphic sites

Specifically, non-designated polymorphisms as contemplated in the present invention may interfere with duplex formation, thereby interfering with or completely inhibiting probe elongation. In one embodiment, the present invention provides designs of covering probe sets to accommodate such non-designated polymorphisms. A covering probe set contains probes for concurrently interrogating a given multiplicity of designated polymorphic sites within a nucleic acid sequence. A covering probe set comprises, for each site, at least one probe capable of annealing to the target so as to permit, on the basis of a subsequent elongation reaction, assignment of one of two possible values to that site: “matched” (elongation) or “unmatched”, (no elongation).

The covering probe set associated with each designated site may contain two or more probes differing in one or more positions, also referred to herein as a degenerate set. In certain embodiments, the probe sequence may contain universal nucleotides capable of forming a base-pair with any of the nucleotides encountered in DNA. In certain embodiments, probes may be attached to encoded microparticles, and specifically, two or more of the probes in a covering set or degenerate set may be attached to the same type of microparticle. The process of attaching two or more probes to a microparticle or bead is referred to as “probe pooling”.

The design of covering probe sets is described herein in connection with elongation-mediated multiplexed analysis of polymorphisms in two representative areas of genetic analysis: (1)[[:]] the scoring of multiple uncorrelated designated polymorphisms and mutations, as in the case of mutation analysis for CF and Ashkenazi Jewish (AJ) disease carrier screening, and (2) the scoring of a correlated set of polymorphisms as in the case of HLA molecular typing. In the first instance, the covering set for the entire multiplicity of mutations contains multiple subsets, each subset being associated with one designated site. In such a case, two or more probes are provided to ascertain heterozygosity. For the purpose of general SNP identification and confirmatory sequencing, degenerate probe sets can be provided to contain up to four labeled (e.g., bead-displayed) probes per polymorphic site. In the second instance, the covering set contains subsets constructed to minimize the number of probes in the set, as elaborated herein. The set of designated probes is designed to identify allele-specific sequence configurations on the basis of the elongation pattern.

While this method of accommodating or identifying non-designated polymorphic sites is especially useful in connection with the multiplexed elongation of sequence specific probes, it also may be used in conjunction with single base extension of probes, also known as mini-sequencing (see e.g., Pastinen, et al. Genome Res. 7: 606-614 (1997), incorporated herein by reference).

The elongation-mediated method of analysis of the present invention, unlike the single-base probe extension method, may be used to detect not only SNPs, but also to detect other types of polymorphisms such as multiple (e.g., double, triple, etc.) nucleotide polymorphisms, as well as insertions and deletions commonly observed in the typing of highly polymorphic genetic loci such as HLA. In these complex systems, sequence-specific probe elongation in accordance with the methods of this invention, simplifies the detection step because two or more probes are provided for each polymorphic target location of interest and the detection step is performed only to determine which of the two or more probes was elongated, rather than to distinguish between two extended probes, as in the case of single-base probe extension. Thus, although the methods of this invention accommodate the use of multiple fluorophore or chromophore labels in the detection step, a single universal label generally will suffice for the sequence specific probe elongation. This is in contrast to single-base extension methods whose application in a multiplexed format requires at least two fluorophore or chromophore labels.

DNA methylation: In certain embodiments, methods and compositions for determining the methylation status of DNA are provided. Cytosine methylation has long been recognized as an important factor in the silencing of genes in mammalian cells. Cytosine methylation at single CpG dinucleotides within the recognition sites of a number of transcription factors is enough to block binding and related to several diseases. eMAP can be used to determine the methylation status of genomic DNA for diagnostic and other purposes. The DNA is modified by sodium bisulfite treatment converting unmethylated Cytosines to Uracil. Following removal of bisulfite and completion of the chemical conversion, this modified DNA is used as a template for PCR. A pair of probes is designed, one specific for DNA that was originally methylated for the gene of interest, and one specific for unmethylated DNA. eMAP is performed with DNA polymerase and one labeled dNTP and unlabeled mixture of 3 dNTPs or ddNTPs. The elongated product on the specific bead surface can indicate the methylation status.

Selective Sequencing: In certain other embodiments of this invention, selective sequencing (also referred to as “sequencing”) is used for concurrent interrogation of an entire set of designated polymorphisms within a nucleic acid sequence in order to determine the composition at each such site. Selective sequencing can be used to provide the requisite information to select, from the set of possible configurations for the sequence of interest, the actual configuration in a given specific sample or to narrow the set of possible sequences in that sample. In selective sequencing, the length of probes used in an extension reaction determine the length of the sequences that can be determined. For longer DNA sequences, staggered probe designs can be used to link the sequences together. Thus, known sequence combinations can be confirmed, while unknown sequence combinations can be identified as new alleles.

Cystic Fibrosis Carrier Screening—One practical application of this invention involves the analysis of a set of designated mutations within the context of a large set of non-designated mutations and polymorphisms in the Cystic Fibrosis Transmembrane Conductance (CFTR) gene. Each of the designated mutations in the set is associated with the disease and must be independently scored. In the simplest case of a point mutation, two encoded probes are provided to ensure alignment of their respective 3′ termini with the designated site, with one probe anticipating the wild-type, and the other anticipating the altered (“mutated”) target sequence.

However, to ensure elongation regardless of the specific target sequence configuration encountered near the designated site, additional probes are provided to match any of the possible or likely configurations, as described in several Example herein. In a preferred embodiment, the covering probe set is constructed to contain probes displaying TEI sequences corresponding to all known or likely variations of the corresponding target subsequence. This ensures elongation in the presence of otherwise elongation-inhibiting non-designated polymorphisms located within a range of proximity of the designated site.

In certain embodiments, the identification of the specific target configuration encountered in the non-designated sites is not necessary so long as one of the sequences provided in the covering probe set matches the target sequence sufficiently closely to ensure elongation, and thus matches the target sequence exactly within the TEI region. In this case, all or some of the covering probes sharing the same 3′ terminus may be assigned the same code In a preferred embodiment, such probes may be associated with the same solid support (“probe pooling”). Probe pooling reduces the number of distinguishable solid supports required to represent the requisite number of TEI sequences. In one particularly preferred embodiment, solid supports are provided in the form of a set or array of distinguishable microparticles which may be decoded in-situ. Inclusion of additional probes in the covering probe set to identify additional polymorphisms in the target region is a useful method to elucidate haplotypes for various populations.

HLA—Another application of this invention involves the genetic analysis of the Human Leukocyte Antigen (HLA) complex, allowing the identification of one or more alleles within regions of HLA encoding class I HLA antigens (preferably HLA-A, HLA-B, HLA-C or any combination thereof) and class II HLA antigens (preferably including HLA-DR, HLA-DQ, HLA-DP or any combination thereof). Class I and II gene loci also may be analyzed simultaneously. In contrast to the independent scoring of multiple uncorrelated designated mutations, identification of alleles (or groups of alleles) relies on the scoring of an entire set of elongation reactions. Each of these reactions involves one or more probes directed to a member of a selected set of designated polymorphic sites. The set of these elongation reactions produces a characteristic elongation signal pattern. In a preferred embodiment, a binary pattern is produced, assigning a value of “1” to matching (and hence elongated) probes, and a value of “0” to non-elongated probes. The binary pattern (“string”) of given length uniquely identifies an allele or a group of alleles.

The total number of probes required for HLA typing depends on the desired resolution. The term “resolution” is used here to indicate the degree of allelic discrimination. Preferably, the method of this invention allows typing of an HLA allele that is sufficient to distinguish different antigen groups. For example, A*01 and A*03 are different antigen groups that have to be distinguished in clinical applications. The National Marrow Donor Program (NMDP) recommended a panel for molecular typing of the donors. The low-to-medium resolution required by the NMDP panel means that different antigen groups should be distinguished at all times. Further, at least some of the alleles within one group should be distinguished, though not necessarily all alleles. In certain embodiments, the present invention allows typing of the HLA allele to a low to medium resolution, as defined by the NMDP standard (www.NMDPresearch.org), incorporated herein by reference.

With such resolution, A*01, A*03 etc., will always be identified. A*0101 and A*0102 may not be necessarily distinguishable. For the SSO method, the current NMDP panel contains 30 probes for HLA-A; 48 for HLA-B and 31 for HLA-DR-B. High resolution HLA typing refers to the situation when most of the alleles will be identified within each group. In this case, A*0101 and A*0102 will be distinguished. To reach such resolution, approximately 500 to 1000 probes will be required for both class I and class II typing. In certain embodiments, the method of the present invention provides high resolution HLA typing, at least to the degree described in Cao, et al., Rev. Immunogenetics, 1: 177-208 (1999), incorporated herein by reference.

This invention also provides strategies for designating sites and for designing probe sets for such designated sites in order to produce unique allele assignments based on the elongation reaction signal patterns. The design of covering probes explicitly takes into account the distinct respective functions of TEI and DA regions of each probe.

A covering set of probes associated with a given designated site is constructed to contain subsets. Each subset in turn contains probes displaying identical TEI regions. A mismatch in a single position within the TEI region, or a mismatch in three or more positions within the DA region precludes elongation. Accordingly, the elongation of two probes displaying such differences in composition generally will produce distinct elongation patterns. All such probes can be multiplexed in a parallel elongation reaction as long as they are individually encoded. In a preferred embodiment, encoding is accomplished by attaching probes to color-encoded beads.

Probes displaying identical TEI subsequences and displaying DA subsequences differing in not more than two positions generally will produce elongation reactions at a yield (and hence signal intensity) either comparable to, or lower than that of a perfect match. In the first case which indicates tolerance of the mismatch, the set of alleles matched by the probe in question will be expanded to include alleles that display the tolerated mismatched sequence configurations within the DA region. In the second case, indicating only partial tolerance, three approaches are described herein to further elucidate the allele matching pattern. In the first approach, probes displaying one or two nucleotide polymorphisms in their respective DA regions are included in the covering set. Information regarding the target sequence is obtained by quantitatively comparing the signal intensities produced by the different probes within the covering set. In the second approach, probes comprising separate TEI and DA regions joined by a tether are used to place the DA region farther away from the TEI region in order to avoid target polymorphisms. In the third approach, probes are optionally pooled in such cases offering only a modest expansion of the set of matched alleles.

In certain embodiments of this invention, probes preferably are designed to be complementary to certain target sequences that are known to correlate with allele combinations within the HLA gene locus. Known polymorphisms are those that have appeared in the literature or are available from a searchable database of sequences (e.g., www.NMDProcessing.org). In certain embodiments, the HLA gene of interest belongs to HLA class I group, (e.g., HLA-A, HLA-B or HLA-C or combination thereof). In certain other embodiments, the HLA gene of interest belongs to the HLA class II group, (e.g., DR, DQ, DP or combination thereof). The HLA class I and class II loci may be examined in combination and by way of concurrent interrogation.

Probes previously employed in the SSP/gel method also may be used in this invention. Preferably, the probes set forth in Bunce et al., Tissue Antigen, 46: 355-367 (1995) and/or Bunce et al., Tissue Antigen, 45:81-90 (1995), (each of which are hereby incorporated by reference) are used in preparing the probes for this invention. The probe sequences or HLA sequence information provided in WO 00/65088; European Application No. 98111696.5; WO 00/70006; and Erlich et al., Immunity, 14: 347-356 (2001), (each of which are hereby incorporated by reference) may be used in designing the probes for this invention.

The complexity of an encoded bead array is readily adjusted to accommodate the requisite typing resolution. For example, when 32 types of beads are used for each of four distinct subarrays, a total of 128 probes will be available to attain a medium level of resolution for HLA class I and class II typing in a multiplexed elongation reaction. Analogously, with 128 types of beads and four subarrays, or 64 types of beads and 8 subarrays, a total of 512 probes will be available to attain a high resolution of HLA class I and class II typing in a multiplexed elongation reaction.

The encoded bead array format is compatible with high throughput analysis. For example, certain embodiments of this invention provide a carrier that accommodates multiple samples in a format that is compatible with the dimensions of 96-well microplates, so that sample distribution may be handled by a standard robotic fluid handling apparatus. This format can accommodate multiple encoded bead arrays mounted on chips and permits the simultaneous completion of multiple typing reactions for each of multiple patient samples on a single multi-chip carrier In a 96-well carrier testing 128 types per patient, more than 10,000 genotypes can be determined at a rate of throughput that is not attainable by current SSP or SSO methodology.

In certain embodiments of this invention, the elongation reaction can be combined with a subsequent hybridization reaction to correlate subsequences on the same DNA target strand, a capability referred to herein as “phasing”. Phasing resolves ambiguities in allele assignment arising from the possibility that a given elongation pattern is generated by different combinations of alleles. Similarly, phasing is useful in the context of haplotyping to assign polymorphisms to the same DNA strand or chromosome.

In certain embodiments of this invention, the annealing and elongation steps of the elongation reaction can be combined as a one-step reaction. Furthermore, means to create continuous or discrete temperature variations can be incorporated into the system to accommodate multiple optimal conditions for probes with different melting temperatures in a multiplexed reaction.

In certain embodiments of this invention; encoded bead arrays are formed on solid substrates. These solid substrates may comprise any suitable solid material, such as glass or semiconductor, that has sufficient mechanical strength and can be subjected to fabrication steps, if desired. In some embodiments, the solid substrates are divided into discrete units known as “chips”. Chips comprising encoded bead arrays may be processed individually or in groups, if they are loaded into a multichip carrier. For example, standard methods of temperature control are readily applied to set the operating temperature of, or to apply a preprogrammed sequence of temperature changes to, single chips or to multichip carriers. Further, chips may be analyzed with the direct imaging capability of Random Encoded Array Detection (“READ”), as disclosed in PCT/US01/20179, the contents of which are incorporated herein by reference. Using READ, the multiplexed analysis of entire arrays of encoded beads on chips is possible. Furthermore, in the READ format, the application of preprogrammed temperature cycles provides real-time on-chip amplification of elongation products. Given genomic, mitochondrial or other DNA, linear on-chip amplification may obviate the need for pre-assay DNA amplification such as PCR, thereby dramatically shortening the time required to complete the entire typing assay. Time-sensitive applications such as cadaver typing are therefore possible. More importantly, this approach eliminates the complexities of PCR multiplexing, which is a limiting step in many genetic screening and polymorphism analyses. In a preferred embodiment, a fluidic cartridge provides for sample and reagent injection as well as temperature control.

In one embodiment, the invention provides a method for polymorphism analysis in which each target nucleic acid sequence is used as a template in multiple elongation reactions by applying one or more “annealing-extending-detecting-denaturing” temperature cycles. This method achieves linear amplification with in-situ detection of the elongation products. This additional capability obviates the need for a first step of sequence-specific amplification of a polynucleotide sample

Integration of assay procedure and signal amplification by way of cycling not only simplifies and accelerates the completion of genetic analysis, but also eliminates the need to develop, test and implement multiplexed PCR procedures. The methods of this invention also provide a high-throughput format for the simultaneous genetic analysis of multiple patient samples.

Several embodiments of this invention are provided for the multiplexed elongation of sequence-specific probes to permit simultaneous evaluation of a number of different targets. In certain embodiments, oligonucleotide probes are immobilized on a solid support to create dense patterns of probes on a single surface, e.g., silicon or glass surface. In certain embodiments, presynthesized oligonucleotide probes are immobilized on a solid support, examples of which include silicon, chemically modified silicon, glass, chemically modified glass or plastic. These solid supports may be in the form of microscopic beads. The resolution of the oligonucleotide array is determined by both spatial resolution of the delivery system and the physical space requirements of the delivered nucleotide solution volume. [See Guo, et al., Nucleic Acids Res. 22: 5456-5465 (1994); Fahy, et al., Nucleic Acid Res. 21: 1819-1826 (1993); Wolf, et al., Nuc. Acids Res. 15: 2911-2926 (1987); and Ghosh, et al., Nuc. Acids Res. 15: 5353-5372 (1987).]

This invention provides methods for multiplexed assays. In certain embodiments, sets of elongation probes are immobilized on a solid phase in a way that preserves their identity, e.g., by spatially separating different probes and/or by chemically encoding the probe identities. One or more solution-borne targets are then allowed to contact a multiplicity of immobilized probes in the annealing and elongation reactions. This spatial separation of probes from one another by immobilization reduces ambiguities in identifying elongation products. Thus, this invention offers advantages over the existing PCR-SSP method, which is not adaptable to a high throughput format because of (i) its requirement for two probes for each PCR amplification; (ii) the competition between overlapping probes for the highly polymorphic genes, such as HLA, in a multiplexed homogeneous reaction; and (iii) the difficulty in distinguishing between specific products in such a multiplexed reaction.

In a preferred embodiment, probes are attached, via their respective 5′ termini, to encoded microparticles (“beads”) having a chemically or physically distinguishable characteristic that uniquely identifies the attached probe. Probes capture target sequences of interest contained in a solution that contacts the beads. Elongation of the probe displayed on a particular bead produces an optically detectable signature or a chemical signature that may be converted into an optically detectable signature. In a multiplexed elongation reaction, the optical signature of each participating bead uniquely corresponds to the probe displayed on that bead. Subsequent to the probe elongation step, one may determine the identity of the probes by way of particle identification and detection, e.g., by flow cytometry.

In certain embodiments, beads may be arranged in a planar array on a substrate before the elongation step. Beads also may be assembled on a planar substrate to facilitate imaging after the elongation step. The process and system described herein provide a high throughput assay format permitting the instant imaging of an entire array of beads and the simultaneous genetic analysis of multiple patient samples.

The array of beads may be a random encoded array, in which a chemically or physically distinguishable characteristic of the beads within the array indicates the identity of oligonucleotide probes attached to the beads. The array may be formed according to the READ format

The bead array may be prepared by employing separate batch processes to produce application-specific substrates (e.g., a chip at the wafer scale). Beads that are encoded and attached to oligonucleotide probes (e.g., at the scale of about 10⁸ beads/100 μl suspension) are combined with a substrate (e.g., silicon chip) and assembled to form dense arrays on a designated area of the substrate. In certain embodiments, the bead array contains 4000 beads of 3.2 μm diameter and has a dimension of 300 μm by 300 μm. With beads of different size, the density will vary. Multiple bead arrays also can be formed simultaneously in discrete fluid compartments maintained on the same chip. Such methods are disclosed in U.S. application Ser. No. 10/192,351, filed Jul. 9, 2002, which is incorporated herein by reference in its entirety.

Bead arrays may be formed by the methods collectively referred to as “LEAPS”, as described in U.S. Pat. No. 6,251,691 and PCT International Application No. PCT/US00/25466), both of which are incorporated herein by reference.

The substrate (e.g., a chip) used in this invention may be in the form of a planar electrode patterned in accordance with the interfacial patterning methods of LEAPS. For example, the substrate may be patterned with oxide or other dielectric materials to create a desired configuration of impedance gradients in the presence of an applied AC electric field. Patterns may be designed so as to produce a desired configuration of AC field-induced fluid flow and corresponding particle transport. Substrates may be patterned on a wafer scale by using semiconductor processing technology. In addition, substrates may be compartmentalized by depositing a thin film of a UV-patternable, optically transparent polymer to affix to the substrate a desired layout of fluidic conduits and compartments. These conduits and compartments confine fluid in one or several discrete compartments, thereby accommodating multiple samples on a given substrate.

Bead arrays may be prepared using LEAPS by providing a first planar electrode that is in substantially parallel to a second planar electrode (“sandwich” configuration) with the two electrodes being separated by a gap and containing a polarizable liquid medium, such as an electrolyte solution. The surface or the interior of the second planar electrode may be patterned with the interfacial patterning method. The beads are introduced into the gap. When an AC voltage is applied to the gap, the beads form a random encoded array on the second electrode (e.g., a “chip”).

In another embodiment of LEAPS, an array of beads may be formed on a light-sensitive electrode (e.g., a “chip”). Preferably, the sandwich configuration described above is also used with a planar light sensitive electrode and another planar electrode. Once again, the two electrodes are separated by the a gap and contain an electrolyte solution. The functionalized and encoded beads are introduced into the gap. Upon application of an AC voltage in combination with light, the beads form an array on the light-sensitive electrode.

In certain embodiments of the present invention, beads may be associated with a chemically or physically distinguishable characteristic. This may be provided, for example, by staining beads with sets of optically distinguishable tags, such as those containing one or more fluorophore or chromophore dyes spectrally distinguishable by excitation wavelength, emission wavelength, excited-state lifetime or emission intensity. The optically distinguishable tags may be used to stain beads in specified ratios, as disclosed, for example, in Fulwyler, U.S. Pat. No. 4,717,655 (Jan. 5, 1988). Staining may also be accomplished by swelling of particles in accordance with methods known to those skilled in the art, (Molday, Dreyer, Rembaum & Yen, J. Mol Biol 64, 75-88 (1975); L. Bangs, “Uniform latex Particles, Seragen Diagnostics, 1984). For example, up to twelve types of beads were encoded by swelling and bulk staining with two colors, each individually in four intensity levels, and mixed in four nominal molar ratios. Alternatively, the methods of combinatorial color encoding described in International Application No. PCT/US 98/10719 (incorporated by reference in its entirety) can be used to endow the bead arrays with optically distinguishable tags. In addition to chemical encoding, beads may also be rendered magnetic by the processes described in PCT/US0/20179.

In addition to chemical encoding with dyes, beads having certain oligonucleotide primers may be spatially separated (“spatial encoding”), such that the location of the beads provides information as to the identity of the beads. Spatial encoding, for example, can be accomplished within a single fluid phase in the course of array assembly by using Light-controlled Electrokinetic Assembly of Particles near Surfaces (LEAPS). LEAPS can be used to assemble planar bead arrays in any desired configuration in response to alternating electric fields and/or in accordance with patterns of light projected onto the substrate.

LEAPS can be used to create lateral gradients in the impedance at the interface between a silicon chip and a solution to modulate the electrohydrodynamic forces that mediate array assembly.

Electrical requirements are modest: low AC voltages of typically less than 10Vₚₚ are applied across a fluid gap between two planar electrodes that is typically 100 μm. This assembly process is rapid and it is optically programmable: arrays containing thousands of beads are formed within seconds under an applied electric field. The formation of multiple subarrays can also occur in multiple fluid phases maintained on a compartmentalized chip surface.

Subsequent to the formation of an array, the array may be immobilized. For example, the bead arrays may be immobilized, for example, by application of a DC voltage to produce random encoded arrays. The DC voltage, set to typically 5-7 V (for beads in the range of 2-6 μm and for a gap size of 100-150 μm) and applied for <30s in “reverse bias” configuration so that an n-doped silicon substrate would form the anode, causes the array to be compressed to an extent facilitating contact between adjacent beads within the array and simultaneously causes beads to be moved toward the region of high electric field in immediate proximity of the electrode surface. Once in sufficiently close proximity, beads are anchored by van der Waals forces mediating physical adsorption. This adsorption process is facilitated by providing on the bead surface a population of “tethers” extending from the bead surface; polylysine and streptavidin have been used for this purpose.

In certain embodiments, the particle arrays may be immobilized by chemical means, e.g, by forming a composite gel-particle film. In one exemplary method for forming such gel-composite particle films, a suspension of microparticles is provided which also contains monomer, crosslinker and initiator for in-situ gel formation. The particles are assembled into a planar assembly on a substrate by using LEAPS. AC voltages of 1-20 Vₚ₋ₚ in a frequency range from 100's of hertz to several kilohertz are applied between the electrodes across the fluid gap. In the presence of the applied AC voltage, polymerization of the fluid phase is triggered after array assembly by thermally heating the cell to ˜40-45° C. using an infra-red (IR) lamp or photoinitiating the reaction using a mercury lamp source. The resultant gel effectively entraps the particle array. Gels may be composed of a mixture of acrylamide and bisacrylamide of varying monomer concentrations from 20% to 5% (acrylamide:bisacrylamide=37.5:1, molar ratio), but any other low viscosity water soluble monomer or monomer mixture may be used as well. Chemically immobilized functionalized microparticle arrays prepared by this process may be used for a variety of bioassays, e.g., ligand receptor binding assays.

In one example, thermal hydrogels are formed using azodiisobutyramidine dihydrochloride as a thermal initiator at a low concentration to ensure that the overall ionic strength of the polymerization mixture falls in the range of ˜0.1 mM to 1.0 mM. The initiator used for the UV polymerization is Irgacure 2959® (2-Hydroxy-4′-hydroxyethoxy-2-methylpropiophenone, Ciba Geigy, Tarrytown, N.Y.). The initiator is added to the monomer to give a 1.5% by weight solution.

In certain embodiments, the particle arrays may be immobilized by mechanical means. For example, an array of microwells may be produced by standard semiconductor processing methods in the low impedance regions of a silicon substrate. Particle arrays may be formed using such structures. In certain embodiments LEAPS mediated hydrodynamic and ponderomotive forces are utilized to transport and to accumulate particles on the hole arrays. The AC field is then switched off and particles are trapped into microwells and thus mechanically confined. Excess beads are removed leaving behind a spatially ordered random bead array on the substrate surface.

Substrates (e.g., chips) can be placed in one or more enclosed compartments that permit samples and reagents to be transported in and out of the compartments through fluidic interconnection. Reactions can also be performed in an open compartment format such as a microtiter plate. Reagents may be pipetted on top of the chip by robotic liquid handling equipment, and multiple samples may be processed simultaneously. Such a format accommodates standard sample processing and liquid handling for the existing microtiter plate format and integrates sample processing and array detection.

In certain embodiments of this invention, encoded beads are assembled on the substrate surface, but not in an array. For example, by spotting bead suspensions into multiple regions of the substrate and allowing beads to settle under gravity, assemblies of beads can be formed on the substrate. In contrast to the bead arrays formed by LEAPS, these assemblies generally assume disordered configurations of low-density or non-planar configurations involving stacking or clumping of beads, thereby preventing imaging of affected beads. However, the combination of spatial and color encoding attained by spotting mixtures of chemically encoded beads into a multiplicity of discrete positions on the substrate still allows multiplexing.

In certain embodiments, a comparison of an image of an array after the assay with a decoded image of the array can be used to reveal chemically or physically distinguishable characteristics, as well as the elongation of probes. This comparison can be achieved by using, for example, an optical microscope with an imaging detector and computerized image capture and analysis equipment. The assay image of the array is taken to detect the optical signature that indicates the probe elongation. The decoded image is taken to determine the chemically and/or physically distinguishable characteristics that uniquely identify the probe displayed on the bead surface. In this way, the identity of the probe on each particle in the array may be identified by a distinguishable characteristic.

Image analysis algorithms may be used in analyzing the data obtained from the decoding and the assay images. These algorithms may be used to obtain quantitative data for each bead within an array. The analysis software automatically locates bead centers using a bright-field image of the array as a template, groups beads according to type, assigns quantitative intensities to individual beads, rejects “blemishes” such as those produced by “matrix” materials of irregular shape in serum samples, analyzes background intensity statistics and evaluates the background-corrected mean intensities for all bead types along with the corresponding variances. Examples of such algorithms are set forth in PCT/US01/20179.

Probe elongation may be indicated by a change in the optical signature, or a change in chemical signature which may be converted to a change in optical signature, originating from the beads displaying elongated probes, for example. Direct and indirect labeling methods well known in the art are available for this purpose. Direct labeling refers to a change in optical signature resulting from the elongation; indirect labeling refers to a change introduced by elongation which requires one or more additional steps to produce a detectable optical signature. In certain embodiments, fluorophore or chromophore dyes may be attached to one of the nucleotides added as an ingredient of probe elongation, such that probe elongation changes the optical signature of beads by changing, for example, fluorescence intensities or by providing other changes in the optical signatures of beads displaying elongation products.

### EXAMPLES

The present invention will be better understood from the Examples which follow. It should be understood that these examples are for illustrative purposes and are not to be construed as limiting this invention in any manner.

### Example 1

### Staggered Probe Design for Multiplexed SSP Analysis

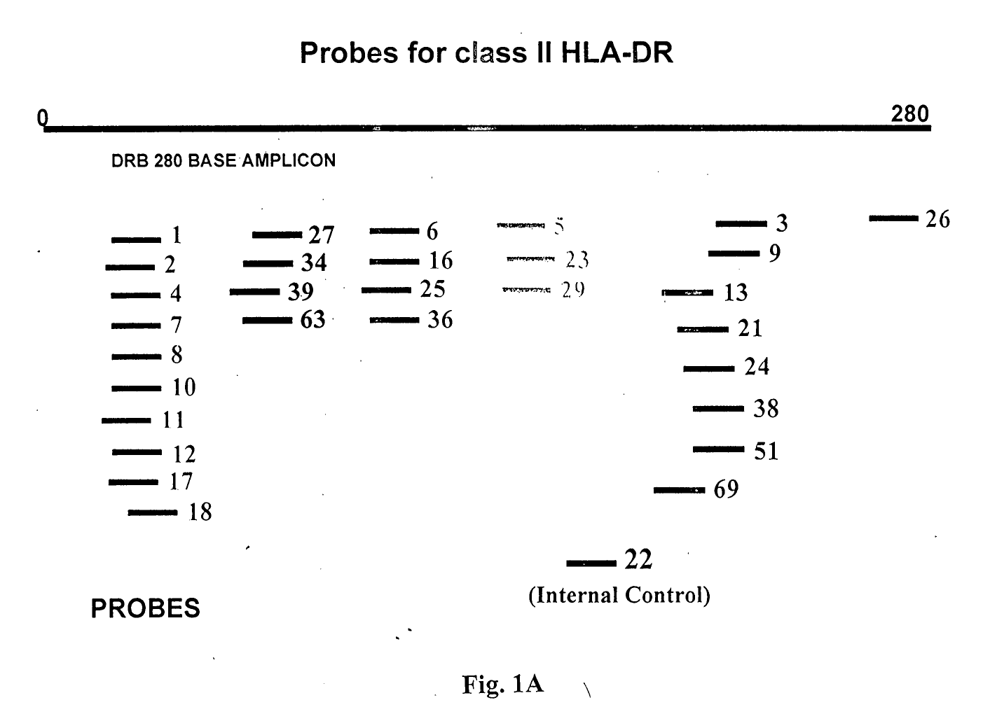

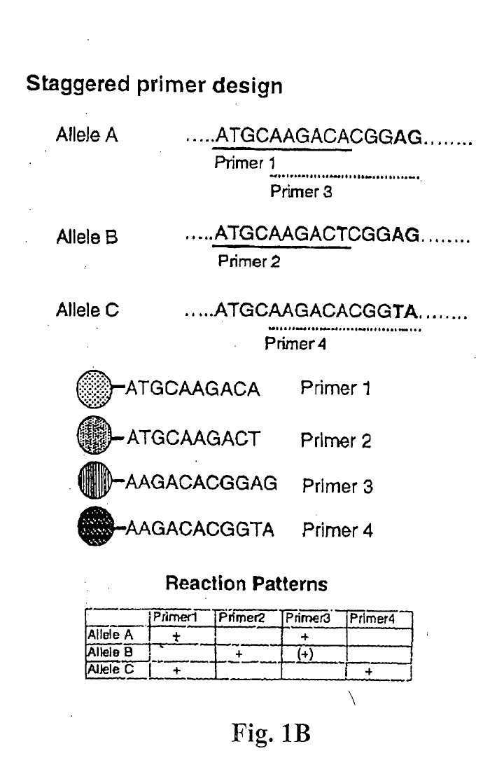

Probes for each polymorphism are immobilized on a solid phase carrier to provide a format in which multiple concurrent annealing and extension reactions can proceed with minimal mutual interference. Specifically, this method provides a design which accommodates overlapping probes, as illustrated in FIG. 1. In this example, we consider three alleles: allele A, allele B and allele C. Probes 1 and 2 detect SNPs that are aligned with their respective 3′ termini while probes 3 and 4 detect two-nucleotide polymorphisms that are aligned with their respective 3′ termini. The polymorphic sites targeted by probes 1 and 2 are located five nucleotides upstream of those targeted by probes 3 and 4. This design permits each probe to bind its corresponding target and permits elongation to proceed when there is a perfect match at the designated polymorphic site. Thus, probes 1 and 3 match allele A, probe 2 and possibly probe 3 match allele B, and probes 1 and 4 match allele C

### Example 2

### Probe Design for HLA Typing

To design probes for the analysis of the polymorphic region ranging from base 106 to base 125 of the DRB gene, twenty-two different types of sequences for the 20 base long fragment were located in the DRB database. These are listed in the table below:

| | | | | |
|---|---|---|---|---|
| 1 | 7 | DRB1*0101 | TTCTTGTGGC | (SEQ ID NO: 1) |
| | | | AGCTTAAGTT | |
| | 104 | DRB1*03011 | TTCTTGGAGT | (SEQ ID NO: 2) |
| | | | ACTCTACGTC | |
| | 26 | DRB1*04011 | TTCTTGGAGC | (SEQ ID NO: 3) |
| | | | AGGTTAAACA | |
| | 1 | DRB1*0434 | TTCTTGGAGC | (SEQ ID NO: 4) |
| | | | AGGTTAAACC | |
| | 3 | DRB1*07011 | TTCCTGTGGC | (SEQ ID NO: 5) |
| | | | AGGGTAAGTA | |
| | 1 | DRB1*07012 | TTCCTGTGGC | (SEQ ID NO: 6) |
| | | | AGGGTAAATA | |
| | 28 | DRB1*0801 | TTCTTGGAGT | (SEQ ID NO: 7) |
| | | | ACTCTACGGG | |
| | 1 | DRB1*0814 | TTCTTGGAGT | (SEQ ID NO: 8) |
| | | | ACTCTAGGGG | |
| | 1 | DRB1*0820 | TTCTTGGAGT | (SEQ ID NO: 9) |
| | | | ACTCTACGGC | |
| | 1 | DRB1*0821 | TTCTTGGAGT | (SEQ ID NO: 10) |
| | | | ACTCTATGGG | |
| | 1 | DRB1*09012 | TTCTTGAAGC | (SEQ ID NO: 11) |
| | | | AGGATAAGTT | |
| | 2 | DRB1*10011 | TTCTTGGAGG | (SEQ ID NO: 12) |
| | | | AGGTTAAGTT | |
| | 1 | DRB1*1122 | TTCTTGGAGC | (SEQ ID NO: 13) |
| | | | AGGCTACACA | |
| | 1 | DRB1*1130 | TTCTTGGAGT | (SEQ ID NO: 14) |
| | | | TCCTTAAGTC | |
| | 18 | DRB1*15011 | TTCCTGTGGC | (SEQ ID NO: 15) |
| | | | AGCCTAAGAG | |
| | 9 | DRB3*01011 | TTCTTGGAGC | (SEQ ID NO: 16) |
| | | | TGCGTAAGTC | |
| | 1 | DRB3*0102 | TTCTTGGAGC | (SEQ ID NO: 17) |
| | | | TGTGTAAGTC | |
| | 1 | DRB3*0104 | TTCTCGGAGC | (SEQ ID NO: 18) |
| | | | TGCGTAAGTC | |
| | 16 | DRB3*0201 | TTCTTGGAGC | (SEQ ID NO: 19) |
| | | | TGCTTAAGTC | |
| | 1 | DRB3*0212 | TTCTTGCAGC | (SEQ ID NO: 20) |
| | | | TGCTTAAGTC | |
| | 6 | DRB4*01011 | TTCTTGGAGC | (SEQ ID NO: 21) |
| | | | AGGCTAAGTG | |
| | 14 | DRB5*01011 | TTCTTGCAGC | (SEQ ID NO: 22) |
| | | | AGGATAAGTA | |

The first column contains the number of alleles sharing the sequence listed in the third column, and the second column contains one of the allele names. We selected the last three bases of the 20-base fragment as the TEI region and sorted the set of sequences according to their TEI region to obtain the following groups:

| | | | | | |
|---|---|---|---|---|---|
| 1 | 104 | DRB1*03011 | TTCTTGGAGT | e1 | (SEQ ID NO: 23) |
| | | | ACTCTACGTC | | |
| | 1 | DRB1*1130 | TTCTTGGAGT | | (SEQ ID NO: 24) |
| | | | gCctTAaGTC | | |
| | 9 | DRB3*01011 | TTCTTGGAGc | | (SEQ ID NO: 25) |
| | | | tgcgTAaGTC | | |
| | 1 | DRB3*0102 | TTCTTGGAGc | | (SEQ ID NO: 26) |
| | | | tgTgTAaGTC | | |
| | 1 | DRB3*0104 | TTCTcGGAGc | | (SEQ ID NO: 27) |
| | | | tgcgTAaGTC | | |
| | 16 | DRB3*0201 | TTCTTGGAGc | e2 | (SEQ ID NO: 28) |
| | | | tgctTAaGTC | | |
| | 1 | DRB3*0212 | TTCTTGcAGc | | (SEQ ID NO: 29) |
| | | | tgctTAaGTC | | |
| 2 | 7 | DRB1*0101 | TTCTTGTGGC | | (SEQ ID NO: 30) |
| | | | AGCTTAAGTT | | |
| | 1 | DRB1*09012 | TTCTTGaaGC | | (SEQ ID NO: 31) |
| | | | AGgaTAAGTT | | |
| | 2 | DRB1*10011 | TTCTTGgaGG | | (SEQ ID NO: 32) |
| | | | AGgTTAAGTT | | |
| 3 | 26 | DRB1*04011 | TTCTTGGAGC | | (SEQ ID NO: 33) |
| | | | AGGTTAAACA | | |
| | 1 | DRB1*1122 | TTCTTGGAGC | | (SEQ ID NO: 34) |
| | | | AGGcTAcACA | | |
| 4 | 1 | DRB1*0434 | TTCTTGGAGC | | (SEQ ID NO: 35) |
| | | | AGGTTAAACC | | |
| 5 | 3 | DRB1*07011 | TTCCTGTGGC | | (SEQ ID NO: 36) |
| | | | AGGGTAAGTA | | |
| | 14 | DRB5*01011 | TTCtTGcaGC | | (SEQ ID NO: 37) |
| | | | AGGaTAAGTA | | |
| 6 | 1 | DRB1*07012 | TTCCTGTGGC | | (SEQ ID NO: 38) |
| | | | AGGGTAAATA | | |
| 7 | 28 | DRB1*0801 | TTCTTGGAGT | e3 | (SEQ ID NO: 39) |
| | | | ACTCTACGGG | | |
| | 1 | DRB1*0814 | TTCTTGGAGT | | (SEQ ID NO: 40) |
| | | | ACTCTAgGGG | | |
| | 1 | DRB1*0821 | TTCTTGGAGT | | (SEQ ID NO: 41) |
| | | | ACTCTAtGGG | | |
| 8 | 1 | DRB1*0820 | TTCTTGGAGT | | (SEQ ID NO: 42) |
| | | | ACTCTACGGC | | |
| 9 | 18 | DRB1*15011 | TTCCTGTGGC | | (SEQ ID NO: 43) |
| | | | AGCCTAAGAG | | |
| 10 | 6 | DRB4*01011 | TTCTTGGAGC | | (SEQ ID NO: 44) |
| | | | AGGCTAAGTG | | |

For sequences in the same group, variations between the first sequence of the group and the rest are indicated in lower case. Three probe sequences are used to illustrate the application of our probe design rules. The first sequence in the first group is selected as probe e1; the 6th sequence in the first group is selected as probe e2; and the first group in the 7th sequence is selected as probe e3.

Due to requirement for perfect complementarity of the target and the probe's TEI region, sequences in group 2 to group 10 do not produce elongation products for e1 and e2. Similarly, sequences in groups other than the 7th group do not produce elongation products for e3. Each group is distinctive from the others with respect to elongation reaction patterns.

For sequences in the same group, there are two types of situations. For example, e1 and e2 differ by one nucleotide in 6 positions within the annealing region. Thus, targets matching e1 and e2 will not produce elongation products for the other sequences, and e1 and e2 are also distinct probes. Similarly, targets for the second to the 7th sequences in group 1 will not produce elongation products for probe e1.

Except for the target matching e1, the remaining 5 sequences only differ from e2 by one or two nucleotides as indicated below:

| | | | | |
|---|---|---|---|---|
| | | 1,2....... | | |
| | | .........M | | |
| 16 | DRB3*0201 | TTCTTGGAGC | e2 | (SEQ ID NO: 45) |
| | | TGCTTAAGTC | | |
| 1 | DRB1*1130 | TTCTTGGAGt | a | (SEQ ID NO: 46) |
| | | TcCTTAAGTC | | |
| 9 | DRB3*01011 | TTCTTGGAGC | b | (SEQ ID NO: 47) |
| | | TGCgTAAGTC | | |
| 1 | DRB3*0102 | TTCTTGGAGC | c | (SEQ ID NO: 48) |
| | | TGtgTAAGTC | | |
| 1 | DRB3*0104 | TTCTcGGAGC | d | (SEQ ID NO: 49) |
| | | TGCgTAAGTC | | |
| 1 | DRB3*0212 | TTCTTGcAGC | e | (SEQ ID NO: 50) |
| | | TGCTTAAGTC | | |

These sequences are cross-reactive. When targets for sequences b and e, which differ from e2 by one base at respective positions M-7 and M-14 anneal to probe e2, the non-designated polymorphism(s) in the annealing region will be tolerated and the elongation reaction will proceed to substantially the same degree as for perfectly matched sequences. When targets for sequences a, c, and d, which differ from e2 by two nucleotides anneal to probe e2, the elongation reaction will exhibit only partial tolerance of the non-designated polymoprhism(s). One approach to improve on this situation is to provide separate probes for a, c, and d, then quantitatively analyze the yield of elongation products by analyzing signals intensities to identify the correct sequences. An alternative would be to bridge the non-designated polymorphisms in the annealing region altogether by adding a physical linker (e.g., a tether) to the e2 probe to be able to separate annealing and TEI regions

For the sequences in the 7th group, the other two sequences will be partially tolerated by the e3 probe. These three sequences may be pooled. The e2 probe will yield elongation products for 30 alleles instead of 28 alleles.

### Example 3

### Utilizing Mismatch Tolerance to Modify Allele Binding Patterns

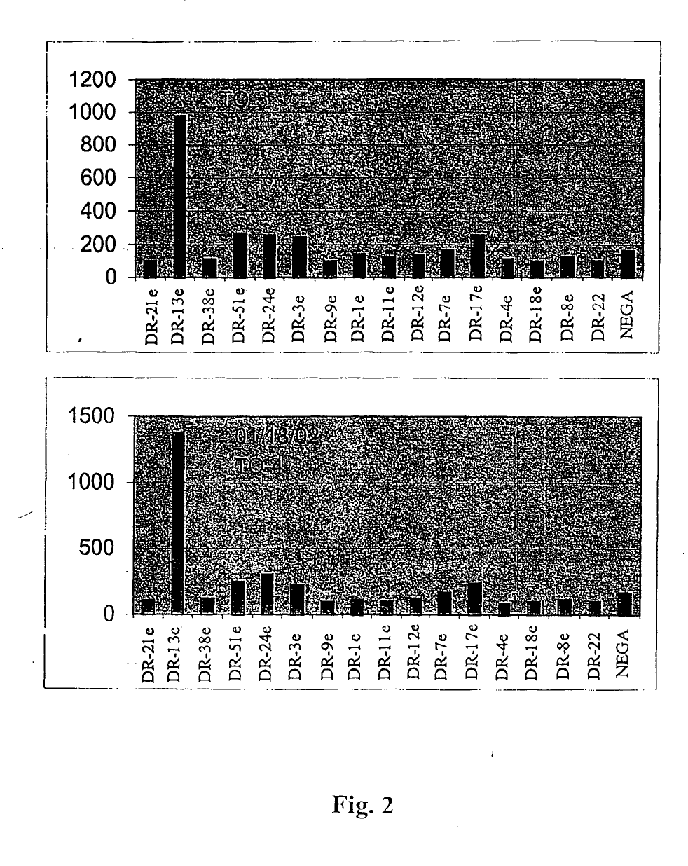

Probe DR-13e, GGACATCCTGGAAGACGA (SEQ ID NO: 51), was used to target the bases 281-299 of the DRB gene. Thirty-four alleles, including allele DRB1*0103, are perfectly matched to this sequence. Thus, in the binding pattern, 13e is positive for theses 34 alleles (that is, 13e will yield elongation products with these 34 alleles). Several additional alleles display the same TEI region but display non-designated polymorphisms in their respective annealing regions. For example, five alleles, such as DRB 1*0415, contain T in instead of A in position 4 while four alleles, such as DRB1*1136, contain C in the that position. Due to mismatch tolerance in the annealing region, target sequences complementary to these nine alleles will produce elongation reaction patterns similar to that of the perfectly matched sequence. The result is shown in FIG. 2. TO-3 and TO-4 are completely complementary sequences to allele *0415 and *1136, respectively.

| | | | |
|---|---|---|---|
| DRB1*0103 | GACATCCTGG | 34 alleles | (SEQ ID NO: 51) |
| | AAGACGA | | |
| DRB1*0415 | GACTTCCTGG | 5 alleles | (SEQ ID NO: 52) |
| | AAGACGA | | |
| DRB1*1136 | GACCTCCTGG | 4 alleles | (SEQ ID NO: 53) |
| | AAGACGA | | |

### Example 4

### Design of Linker Structure in the Probes to Bridge Non-designated Polymorphisms

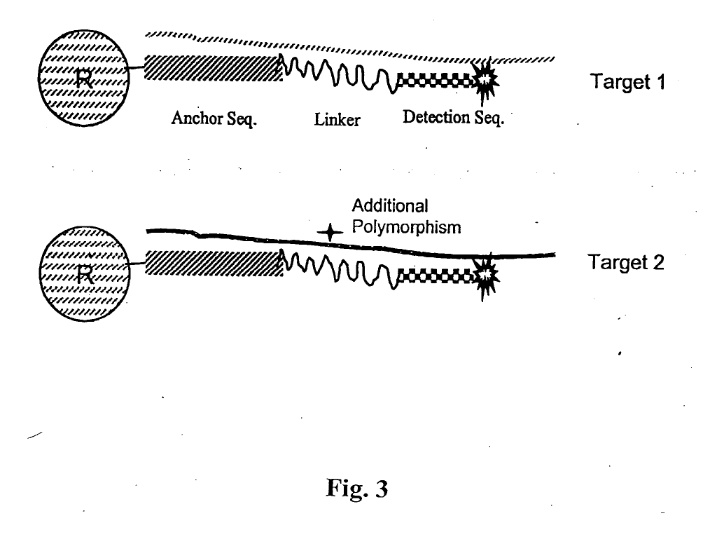

As illustrated in FIG. 3, an anchor sequence is derived from conserved sequence regions to ensure specific and strong annealing. It is not designed for polymorphism detection. For that purpose, a shorter sequence for polymorphism detection is attached to the anchoring sequence by way of a neutral chemical linker. The shorter length of the sequence designed for polymorphism detection will limit potential interference to non-designated polymorphisms in the immediate vicinity of the designated site and thus decreases the number of possible sequence combinations required to accommodate such interfering polymorphisms. This approach avoids highly dense polymorphic sites in certain situations. For example, it would be possible to distinguish between the sequences listed in Example 3 using a probe which takes into account the additional polymorphism(s). Illustrative designs of the linker and the sequences are listed below:

| | | |
|---|---|---|
| linker 13-5 | AGCCAGAAGGAC/ | (SEQ ID NO: 54) |
| | Spacer 18/spacer 18/ | |
| | GGAAGACGA | |
| linker 13-8 | AGCCAGAAGGAC/ | (SEQ ID NO: 54) |
| | Spacer 18/spacer 18/ | |
| | AGACGA | |
| linker 13-11 | AGCCAGAAGGAC/ | (SEQ ID NO: 54) |
| | Spacer 18/spacer 18/ | |
| | CGA | |

### Example 5

### Phasing

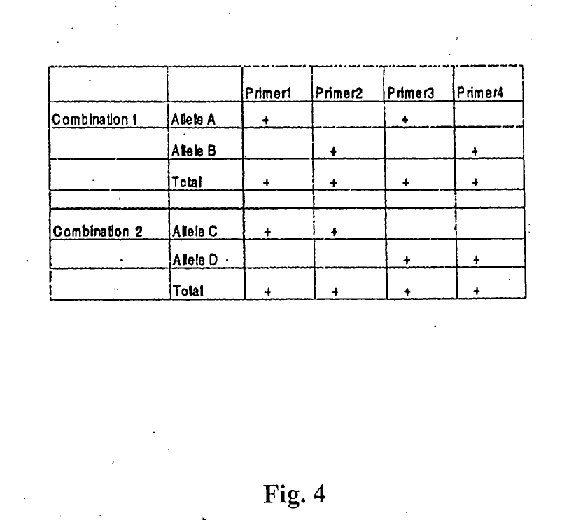

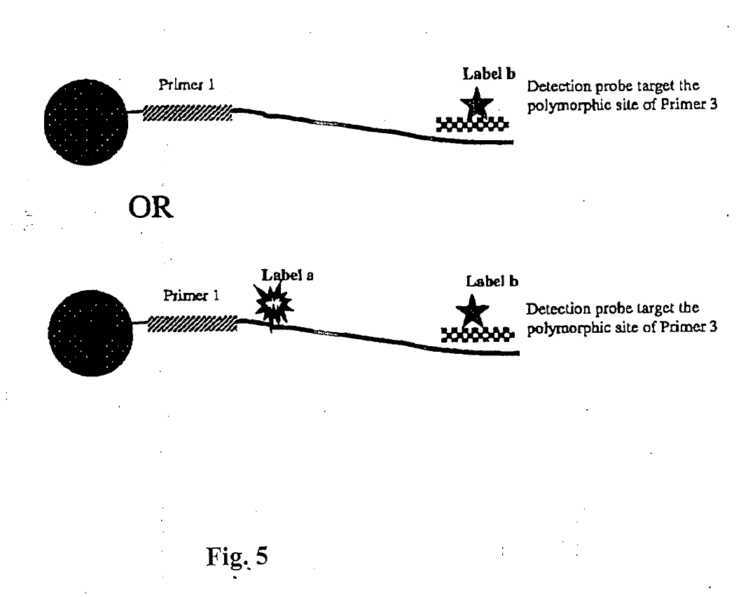

The present invention also is useful in reducing ambiguities that arise when two or more allele combinations can produce the same reaction pattern. In a simulated situation shown in FIGS. 4 and 5, allele A which matches—and hence produces an elongation product with—Probe 1 and Probe 3, and allele B, which matches Probe 2 and Probe 4 when present in the same multiplexed reaction, generate the same total reaction pattern as does the combination of allele C which matches Probe 1 and 2, and allele D which matches Probe 3 and Probe 4. Such ambiguity can be reduced or eliminated by using the detection methods provided in this invention to analyze the elongation product of Probe 1 by hybridization using a labeled detection probe that is designed to target the same polymorphic site as Probe 3. If the result of the analysis is positive, only one allele combination, namely combination 1, is possible because Probe I and Probe 3 are associated with the same allele. The detection probe can be labeled by using any of the methods disclosed in this invention or methods known in the art. If this identification detection step is performed together with the multiplexed elongation reaction detection, different labels are used for the elongation detection and probe hybridization detection as shown in the FIG. 5.

In this method, the ambiguity is resolved by assigning two or more polymorphisms to the same “phase” using elongation in conjunction with hybridization. Phasing is rapidly emerging as an important concern for haplotype analysis in other genetic studies designed in the art. More probes can be included by reacting them with the target sequentially, or they can be arranged in the same reaction with different labels for detection.

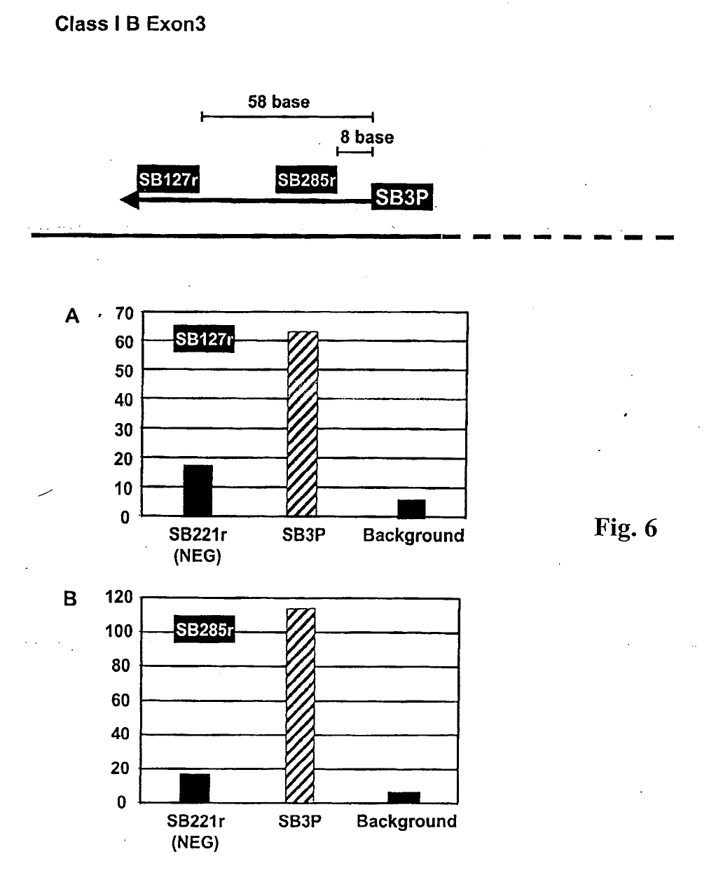

The capability of combining probe elongation and hybridization reactions is demonstrated in experiments using a sample sequence from HLA-B exon 3. The result is shown in FIG. 6. A probe SB3P was elongated in the reaction and the elongated product was detected using a labeled DNA probe. For the two samples presented in FIGS. 6A and 6B, SB 127r and SB3P, and SB285r and SB3P are in the same phase, respectively.

### Example 6

### Model HLA Typing Reaction Using Random Encoded Probe Arrays

To illustrate the discrimination of polymorphisms, a model reaction was performed using a synthetic single strand as the target. Color encoded, tosyl-functionalized beads of 3.2 μm diameter were used as solid phase carriers. A set of 32 distinguishable color codes was generated by staining particles using standard methods known in the art (Bangs. L. B., “Uniform Latex Particles”, Seragen Diagnostics Inc., p. 40) and using different combinations of blue dye (absorption/emission 419/466 nm) and green dye (absorption/emission 504/511). Stained beads were functionalized with Neutravidin (Pierce, Rockford, Ill.), a biotin binding protein, to mediate immobilization of biotinylated probes. In a typical small-scale coupling reaction, 200 μl of suspension containing 1% beads were washed three times with 500 μl of 100 mM phosphate buffer/pH 7.4 (buffer A) and resuspended in 500 μl of that buffer. After applying 20 μl of 5 mg/ml neutravidin to the bead suspension, the reaction was sealed and allowed to proceed overnight at 37° C. Coupled beads were then washed once with 500 μl of PBS/pH 7.4 with 10 mg/ml BSA (buffer B), resuspended in 500 μl of that buffer and reacted for 1 hour at 37° C. to block unreacted sites on bead surface. After blocking, beads were washed three times with buffer B and stored in 200 μl of that buffer.

In the model reaction system, two pairs of probes were synthesized to contain SNPs at their respective 3′ termini. The respective sequences were as follows:

| | | |
|---|---|---|
| SSP13: | AAGGACATCCTGGAAGACG; | (SEQ ID NO: 55) |
| SSP24: | AAGGACATCCTGGAAGACA; | (SEQ ID NO: 56) |
| SSP16: | ATAACCAGGAGGAGTTCC; | (SEQ ID NO: 57) |
| SSP36: | ATAACCAGGAGGAGTTCG. | (SEQ ID NO: 58) |

The probes were biotinylated at the 5′ end; a 15-carbon triethylene glycol linker was inserted between biotin and the oligonucleotide to minimize disruptive effects of the surface immobilization on the subsequent reactions. For each probe, coupling to encoded beads was performed using 50 μl of bead suspension. Beads were washed once with 500 μl of 20 mM Tris/pH 7.4, 0.5M NaCl (buffer C) and resuspended in 300 μl of that buffer. 2.5 μl of a 100 μM solution of probe were added to the bead suspension and allowed to react for 30 min at room temperature. Beads were then washed three times with 20 mM Tris/pH7.4, 150 mM NaCl, 0.01% triton and stored in 20 mM Tris/pH 7.4, 150 mM NaCl.

The following synthetic targets of 33 bases in length were provided:

| | | |
|---|---|---|
| TA16: | GTCGAAGCGCAGGAACTCCT | (SEQ ID NO: 59) |
| | CCTGGTTATGGAA | |
| TA36: | GTCGAAGCGCACGAACTCCT | (SEQ ID NO: 60) |
| | CCTGGTTATAGAA | |
| TA13: | GGCCCGCTCGTCTTCCAGGA | (SEQ ID NO: 61) |
| | TGTCCTTCTGGCT | |
| TA24: | GGCCCGCTTGTCTTCCAGGA | (SEQ ID NO: 62) |
| | TGTCCTTCTGGCT | |

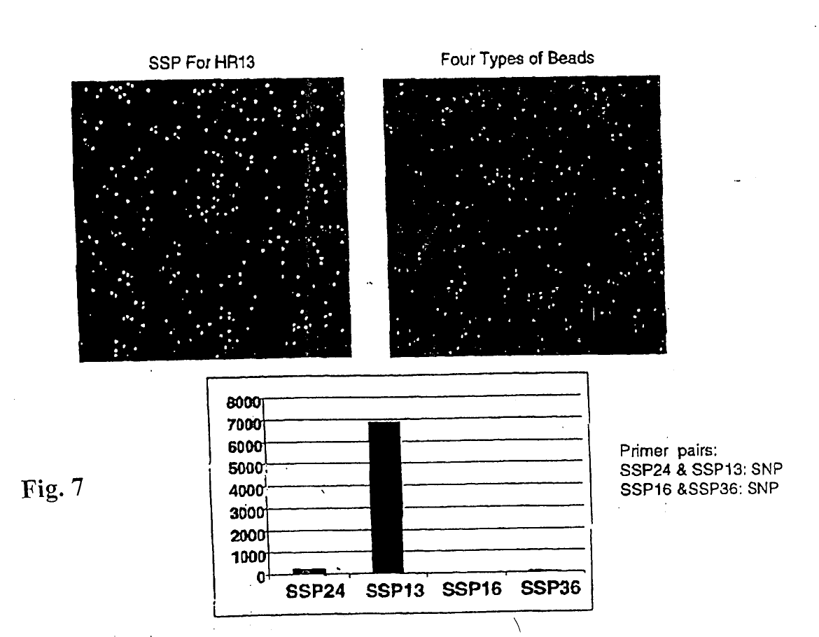

Targets were allowed to react with four probes (SSP13, SSP24, SSP16, SSP36) on the chip. An aliquot of 10 μl of a 100 nM solution of the target in annealing buffer of 0.2 M NaCl, 0.1% Triton X-100, 10 mM Tris/pH 8.0, 0.1 mM EDTA was applied to the chip and allowed to react for 15 min at 30° C. The chip was then washed once with the same buffer and was then covered with an extension reaction mixture including: 100 nM of TAMRA-ddCTP (absorption/emission: 550/580) (PerkinElmer Bioscience, Boston, Mass.), 10 μM dATP-dGTP-dTTP, ThermoSequenase (Amersham, Piscataway, N.J.) in the associated buffer supplied by the manufacturer. The reaction was allowed to proceed for 5 min at 60° C., and the chip was then washed in H₂O. Decoding and assay images of the chip were acquired using a Nikon fluorescence E800 microscope with an automated filter changer containing hydroxy coumarin, HQ narrow band GFP and HQ Cy3 filters for blue, green decoding images and for the assay image, respectively. An Apogee CCD KX85 (Apogee Instruments, Auburn, Calif.) was used for image acquisition. In each reaction, only the perfectly matching target was extended producing, in the case of the SNPs tested here, discrimination between matching and non-matching targets in the range from 13-fold to 30-fold; this is illustrated in FIG. 7 for TA13.

### Example 7

### HLA-DR Typing of Patient Sample

A DNA sample extracted from a patient was processed using a standard PCR protocol. The following primers were used for general DR amplification:

| | | |
|---|---|---|
| forward primer: | GATCCTTCGTGT | (SEQ ID NO: 63) |
| | CCCCACAGCACG | |
| reverse primer: | GCCGCTGCACTG | (SEQ ID NO: 64) |
| | TGAAGCTCTC | |

The PCR protocol was as follows: one cycle of 95° C. for 7 min, 35 cycles of 95° C. for 30 sec, 60° C. for 30 sec and 72° C. for 1 min and one cycle of 72° C. for 7 min.

The PCR product, 287 bases in length and covering the DR locus, was denatured at 100° C. for 5 min, chilled on ice and mixed with annealing buffer as described in Example 6 for the model reaction. An aliquot of 10 ul was applied to each chip and reacted at 40° C. for 15 min. The elongation reaction and subsequent image acquisition proceeded as in the previous Example 6.

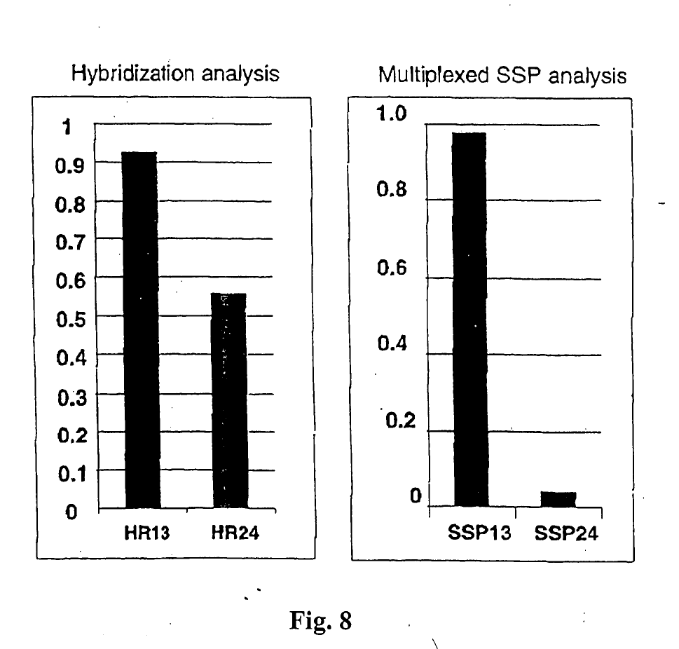

The multiplexed extension of sequence-specific probes using the PCR product produced from the patient sample produced results in accordance with the probe design. Of the four probes tested in parallel (SSP13, SSP16, SSP24, SSP36), SSP13 was elongated while the SNP probe SSP24 only showed background binding as did the unrelated SSP16 and SSP36 probes. As illustrated in FIG. 8, the multiplexed elongation of SSP significantly enhanced the discrimination between matching and non-matching SNPs from approximately two-fold for an analysis based on the hybridization of matching and non-matching sequence-specific oligonucleotide probes to at least 20-fold.

### Example 8

### Group-Specific Amplification

Primers for group-specific amplification (GSA) are most frequently used when multiplexed hybridization with SSOs yields ambiguous assignments of heterozygous allele combinations. In such a situation, GSA primers are selected to amplify selected sets of specific alleles so as to remove ambiguities, a labor-intensive additional assay step which delays the analysis. Using the methods of the present invention, preferably an embodiment of displaying probes on random encoded bead arrays, GSA primers may be incorporated as probes into the multiplexed reaction thereby eliminating an entire second step of analysis.

### Example 9

### Analysis of HLA-DR, -A and -B Loci Using Cell Lines

Probes for the elongation-mediated multiplexed analysis of HLA-DR, HLA-A and HLA-B were designed and tested using standard cell lines. The probes were derived from SSP probes previously reported in the literature (Bunce, M. et al, Tissue Antigens. 46:355-367 (1995), Krausa, P and Browning, M. J., Tissue Antigens. 47: 237-244 (1996), Bunce, M. et al, Tissue Antigens. 45:81-90 (1995)).

The probes used for DR were:

| | | |
|---|---|---|
| SR2: | ACGGAGCGGGTGCGGTTG | (SEQ ID NO: 65) |
| SR3: | GCTGTCGAAGCGCACGG | (SEQ ID NO: 66) |
| SR11: | CGCTGTCGAAGCGCACGTT | (SEQ ID NO: 67) |
| SR19: | GTTATGGAAGTATCTGTCCAGGT | (SEQ ID NO: 68) |
| SR23: | ACGTTTCTTGGAGCAGGTTAAAC | (SEQ ID NO: 69) |
| SR32: | CGTTTCCTGTGGCAGGGTAAGTATA | (SEQ ID NO: 70) |
| SR33: | TCGCTGTCGAAGCGCACGA | (SEQ ID NO: 71) |
| SR36: | CGTTTCTTGGAGTACTCTACGGG | (SEQ ID NO: 72) |
| SR39: | TCTGCAGTAGGTGTCCACCA | (SEQ ID NO: 73) |
| SR45: | CACGTTTCTTTGGAGCTGCG | (SEQ ID NO: 74) |
| SR46: | GGAGTACCGGGCGGTGAG | (SEQ ID NO: 75) |
| SR48: | GTGTCTGCAGTAATTGTCCACCT | (SEQ ID NO: 76) |
| SR52: | CTGTTCCAGGACTCGGCGA | (SEQ ID NO: 77) |
| SR57: | CTCTCCACAACCCCGTAGTTGTA | (SEQ ID NO: 78) |
| SR58: | CGTTTCCTGTGGCAGCCTAAGA | (SEQ ID NO: 79) |
| SR60: | CACCGCGGCCCGCGC | (SEQ ID NO: 80) |
| SR67: | GCTGTCGAAGCGCAAGTC | (SEQ ID NO: 81) |
| SR71: | GCTGTCGAAGCGCACGTA | (SEQ ID NO: 82) |
| NEG | AAAAAAAAAAAAAAAAAA | (SEQ ID NO: 83) |

Some of the probes have a SNP site at their respective 3′ termini, for example: SR3 and SR33 (G and A, respectively); SR11, SR67 and SR71 (T, C, and A, respectively). In addition, probes SR3 and 33 are staggered at the 3′-end with respect to probes the group of SR11, 67 and 71 by one base.

| | | |
|---|---|---|
| SR3 | GCTGTCGAAGCGCACGG | (SEQ ID NO: 84) |
| SR33 | TCGCTGTCGAAGCGCAGGA | (SEQ ID NO: 85) |
| SR11 | CGCTGTCGAAGCGGACGTT | (SEQ ID NO: 86) |
| SR67 | GCTGTCGAAGCGCAAGTC | (SEQ ID NO: 87) |
| SR71 | GCTGTCGAAGCGCACGTA | (SEQ ID NO: 88) |

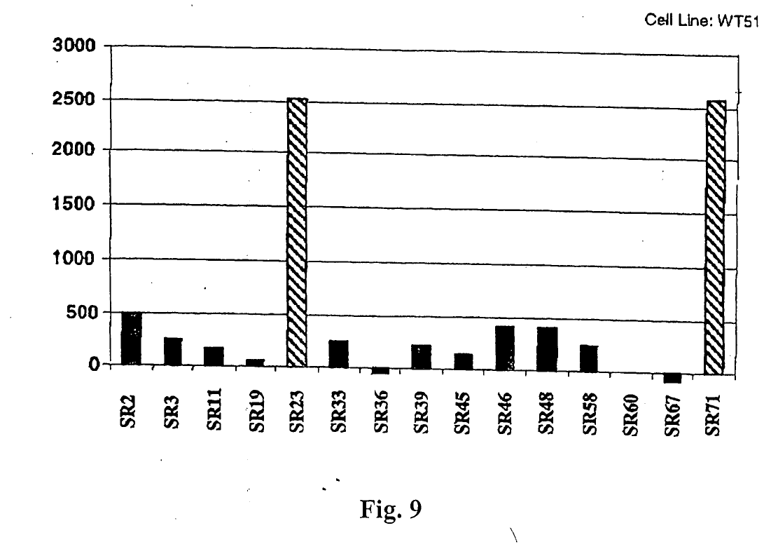

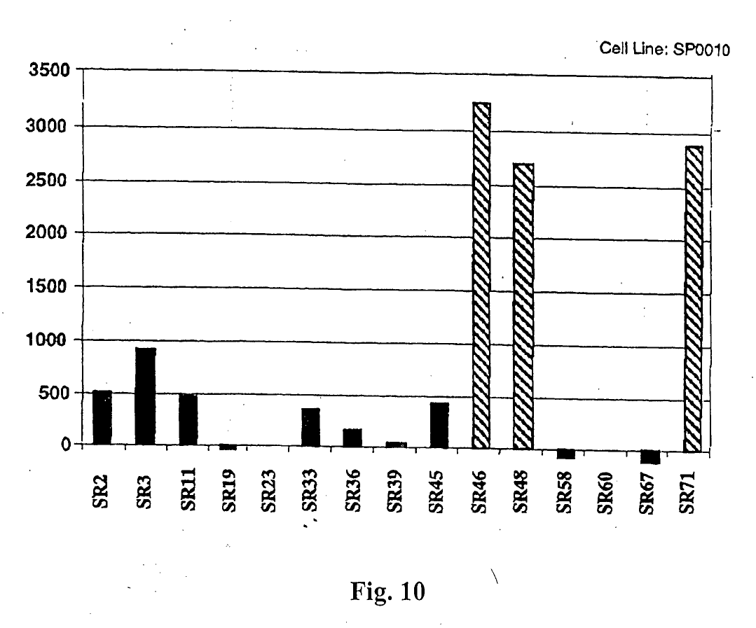

Reaction conditions were as described in Example 7 except that the annealing temperature was 55° C. instead of 40° C., and the extension temperature was 70° C. instead of 60° C. Double-stranded DNA was used as in Example 7. Single-stranded DNA generated better results under current conditions. Single-stranded DNA was generated by re-amplifying the initial PCR product in the same PCR program with only one of the probes. Results for two cell lines, W51 and SP0010, are shown in FIG. 9 and FIG. 10. NEG, a negative control, was coupled to a selected type of bead. Signal intensity for other probes minus NEG was considered to be real signal for the probe and the values were plotted in the figures. The Y axis unit was the signal unit from the camera used in the experiment. The distinction between the positive and negative probes was unambiguous for each sample. In particular, and in contrast to the situation typically encountered in SSO analysis, it was not necessary to make comparisons to other samples to determine a reliable threshold for each probe.

The probes used for HLA-A were:

| | | |
|---|---|---|
| SAD | CACTCCACGCACGTGCCA | (SEQ ID NO: 89) |
| SAF | GCGCAGGTCCTCGTTCAA | (SEQ ID NO: 90) |
| SAQ | CTCCAGGTAGGCTCTCAA | (SEQ ID NO: 91) |
| SAR | CTCCAGGTAGGCTCTCTG | (SEQ ID NO: 92) |
| SAX | GCCCGTCCAGGCACCG | (SEQ ID NO: 93) |
| SAZ | GGTATCTGCGGAGCCCG | (SEQ ID NO: 94) |
| SAAP | CATCCAGGTAGGCTCTCAA | (SEQ ID NO: 95) |
| SA8 | GCCGGAGTATTGGGACGA | (SEQ ID NO: 96) |
| SA13 | TGGATAGAGCAGGAGGGT | (SEQ ID NO: 97) |
| SA16 | GACCAGGAGACACGGAATA | (SEQ ID NO: 98) |

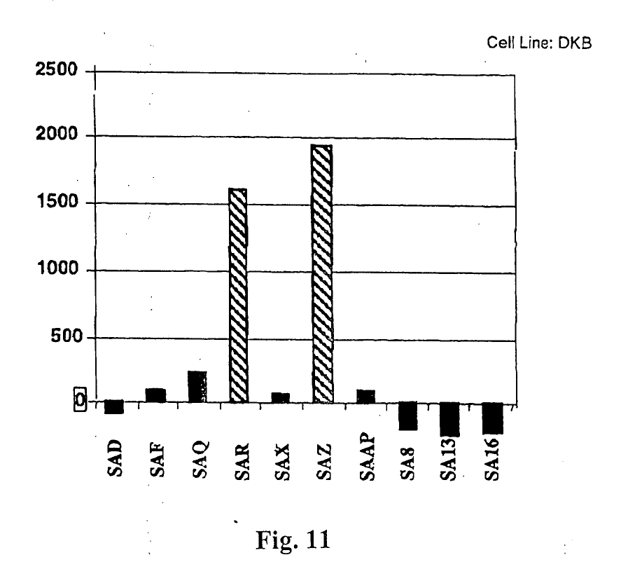

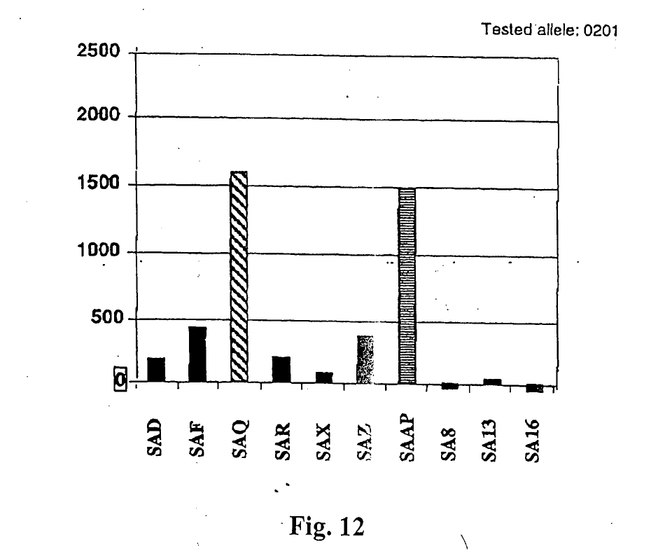

Results for A locus exon 3, shown in FIG. 11 and FIG. 12, also were unambiguous. FIG. 12 also shows an example of the mismatch tolerance for a non-designated polymorphism. That is, while allele 0201, displaying C instead of A at position M-18, is not perfectly matched to probe SAAP, the elongation reaction nonetheless proceeded because the polymerase detected a perfect match for the designated polymorphism at the probe's 3′ end and tolerated the mismatch at position M-18.

The probes used for HLA-B were:

| | | |
|---|---|---|
| SB220 | CCGCGCGCTCCAGCGTG | (SEQ ID NO: 99) |
| SB246 | CCACTCCATGAGGTATTTCC | (SEQ ID NO: 100) |
| SB229 | CTCCAACTTGCGCTGGGA | (SEQ ID NO: 101) |
| SB272 | CGCCACGAGTCCGAGGAA | (SEQ ID NO: 102) |
| SB285 | GTCGTAGGCGTCCTGGTC | (SEQ ID NO: 103) |
| SB221 | TACCAGCGCGCTCCAGCT | (SEQ ID NO: 104) |
| SB197 | AGCAGGAGGGGCCGGAA | (SEQ ID NO: 105) |
| SB127 | CGTCGCAGCCATACATCCA | (SEQ ID NO: 106) |
| SB187 | GCGCCGTGGATAGAGCAA | (SEQ ID NO: 107) |
| SB188 | GCCGCGAGTCCGAGGAC | (SEQ ID NO: 108) |
| SB195 | GACCGGAACACACAGATCTT | (SEQ ID NO: 109) |

Experiments using these probes for typing HLA-B exon 2 were performed using reference cell lines. As with HLA-A, unambiguous results (not shown here) were obtained.

### Example 10

### CF Mutation Analysis—Probe and Array Design for Probe Elongation

This Example describes the design and application of a planar array of probes, displayed on color-encoded particles, these probes designed to display several-most frequently two selected base compositions at or near their respective 3′ ends and designed to align with designated regions of interest within the CFTR target gene.

The CFTR gene sequence from Genebank (see the website of the National Center for Biotechnology Information, National Library of Medicine, National Institute of Health www.ncbi.nlm.nih.gov) was used to design sixteen-mer probes for the multiplexed analysis of the 25 CFTR mutations in the ACMG-CF mutation panel. Probe sequences were designed using PROBE 3.0 (see the website of the Broad Institute http://www.genome.wi.mit.edu) and aligned with respective exon sequences (Baylor College of Medicine Search Launcher: Pairwise Sequence Alignment (available online) http://searchlauncher.bcm.tmc.edu/seq search alignment.html). Oligonucleotides were designed to comprise 15 to 21 nucleotides, with a 30-50% G+C rich.base composition and synthesized to contain a 5′ biotin TEG (Synthegen TX); to handle small deletions, the variable sequence of the TEI region was placed at or within 3-5 positions of the probe's 3′ terminus. Probe compositions are listed in the table below.

A combination of 17 either pure blue or blue-green stained beads were used with CF mutation analysis. The 48-base-long Human .beta.-actin gene (Accession #X00351) was synthesized and used in each reaction as an internal positive control. Sixteen-base-long complementary probes were included on each array. The CFTR gene sequence from Genebank (www.ncbi.nlm.nih.gov) was used for probe design for analysis of 25 CFTR mutations in the ACMG-CF mutation panel. The probe sequences were designed by PROBE 3.0 (see the website of the Broad Institute http://www.genome.wi.mit.edu). Each probe sequence was aligned with respective exon sequences (Baylor College of Medicine Search Launcher: Pairwise Sequence Alignment (available online) http://searchlauncher.bcm.tmc.edu/seq search/alignment.html). Oligonucleotides were synthesized with a 5′ biotin TEG (Synthegen Tex.) and coupled on the surface of beads in presence of 0.5 M NaCl. Bead's were immobilized on the surface of a chip by LEAPS.

| EXON MUTATIONS SEQUENCE | | | |
|---|---|---|---|
| 3 | G85E | CCC CTA AAT ATA AAA AGA TTC | (SEQ ID NO: 110) |
| | G85E-X | CCC CTA AAT ATA AAA AGA TTT | (SEQ ID NO: 111) |
| 4 | 1148 | ATT CTC ATC TCC ATT CCA A | (SEQ ID NO: 112) |
| | 1148-X | ATT CTC ATC TCC ATT GCA G | (SEQ ID NO: 113) |
| | 621 + 1G>T | TGT GTG CAA GGA AGT AAT AC | (SEQ ID NO: 114) |
| | 621 + 1G>T − X | TGT GTG CAA GGA AGT ATT AA | (SEQ ID NO: 115) |
| | R117H | TAG ATA AAT CGC GAT AGA GC | (SEQ ID NO: 116) |
| | R117H-X | TAG ATA AAT CGC GAT AGA GT | (SEQ ID NO: 117) |
| 5 | 711 + 1G>T | TAA ATC AAT AGG TAC ATA G | (SEQ ID NO: 118) |
| | | TAA ATC AAT AGG TAC ATA A | (SEQ ID NO: 119) |
| 7 | R334W | ATG GTG GTG AAT ATT TTC CG | (SEQ ID NO: 120) |
| | R334W-X | ATG GTG GTG AAT ATT TTC CA | (SEQ ID NO: 121) |
| | R347P | ATT GCC GAG TGA CCG CCA TGC | (SEQ ID NO: 122) |
| | R347P-X | ATT GCC GAG TGA CCG CCA TGG | (SEQ ID NO: 123) |
| | 1078delT | CAC AGA TAA AAA CAC CAC AAA | (SEQ ID NO: 124) |
| | 1078delT-X | CAC AGA TAA AAA CAC CAC AA | (SEQ ID NO: 125) |
| | 1078delT-X-2 | CAC AGA TAA AAA CAC CAC A | (SEQ ID NO: 126) |
| 9 | A455E | TCC AGT GGA TCC AGC AAC CG | (SEQ ID NO: 127) |
| | A455E-X | TCC AGT GGA TCC AGC AAC CT | (SEQ ID NO: 128) |
| 10 | 508 | CAT AGG AAA CAC CAA AGA T | (SEQ ID NO: 129) |
| | 1507 | CAT AGG AAA CAC CAA A | (SEQ ID NO: 130) |
| | F508 | CAT AGG AAA CAC CAA T | (SEQ ID NO: 131) |
| 11 | 1717 − 1G>A | CTG CAA ACT TGG AGA TGT CC | (SEQ ID NO: 132) |
| | 1717 − 1G>A | CTG CAA ACT TGG AGA TGT CT | (SEQ ID NO: 133) |
| | 551D | TTC TTG CTC GTT GAC | (SEQ ID NO: 134) |
| | 551D-X | TTC TTG CTC GTT GAT | (SEQ ID NO: 135) |
| | R553 | TAAAGAAATTCTTGCTCG | (SEQ ID NO: 136) |
| | R553X | TAAAGAAATTCTTGCTCA | (SEQ ID NO: 137) |
| | R560 | ACCAATAATTAGTTATTCACC | (SEQ ID NO: 138) |
| | R560X | ACCAATAATTAGTTATTCACG | (SEQ ID NO: 139) |
| | G542 | GTGTGATTCCACCTTCTC C | (SEQ ID NO: 140) |
| | G542X | GTGTGATTCCACCTTCTC A | (SEQ ID NO: 141) |
| INT-12 | 1898 | AGG TAT TCA AAG AAC ATA C | (SEQ ID NO: 142) |
| | 1898-X | AGG TAT TCA AAG AAC ATA T | (SEQ ID NO: 143) |
| 13 | 2183deLA | TGT CTG TTT AAA AGA TTG T | (SEQ ID NO: 144) |
| | 2183deLA-X | TGT CTG TTT AAA AGA TTG C | (SEQ ID NO: 145) |
| INT 14B | 2789 | CAA TAG GAC ATG GAA TAC | (SEQ ID NO: 146) |
| | 2789-X | CAA TAG GAC ATG GAA TAC T | (SEQ ID NO: 147) |
| NT 16 | 3120 | ACT TAT TTT TAC ATA C | (SEQ ID NO: 148) |
| | 3120-X | ACT TAT TTT TAC ATA T | (SEQ ID NO: 149) |
| 18 | D1152 | ACT TAC CAA GCT ATC CAC ATC | (SEQ ID NO: 150) |
| | D1152 | ACT TAC CAA GCT ATC CAC ATG | (SEQ ID NO: 151) |
| INT 19 | 3849 + 10 kbC>T-WT1 | CCT TTC Agg GTG TCT TAC TCG | (SEQ ID NO: 152) |
| | 3849 + 10 kbG>T-M1 | CCT TTC Agg GTG TCT TAC TCA | (SEQ ID NO: 153) |
| 19 | R1162 | AAT GAA CTT AAA GAC TCG | (SEQ ID NO: 154) |
| | R1162-X | AAT GAA CTT AAA GAC TCA | (SEQ ID NO: 155) |
| | 3659delC-WT1 | GTA TGG TTT GGT TGA CTT GG | (SEQ ID NO: 156) |
| | 3659delCX-M1 | GTA TGG TTT GGT TGA CTT GTA | (SEQ ID NO: 157) |
| | 3659delC-WT2 | GTA TGG TTT GGT TGA CTT GGT A | (SEQ ID NO: 158) |
| | 3659delCX-M2 | GTA TGG TTT GGT TGA CTT GT A | (SEQ ID NO: 159) |
| 20 | W1282 | ACT CCA AAG GCT TTC CTC | (SEQ ID NO: 160) |
| | W1282-X | CTC CAA AGG CTT TCC TT | (SEQ ID NO: 161) |
| 21 | N1303K | TGT TCA TAG GGA TCC AAG | (SEQ ID NO: 162) |
| | N1303K-X | TGT TCA TAG GGA TCC AAC | (SEQ ID NO: 163) |
| b | .beta. Actin | AGG ACT CCA TGC CCA G | (SEQ ID NO: 164) |

Probes were attached, in the presence of 0.5 M NaCl, to differentially encoded beads, stained either pure blue or blue-green Beads were immobilized on the surface of a chip using LEAPS. A synthetic 48 base Human β-actin gene (Accession #X00351) was included in each reaction as an internal positive control.

Array Design—In a preferred embodiment, the 25 CF mutations were divided into four different groups so as to minimize sequence homologies between members of each group. That is, mutations were sorted into separate groups so as to minimize overlap between probe sequences in any such group and thereby to minimize cross-hybridization under conditions of multiplexed analysis. Each group, displayed on color-encoded beads, was assembled into a separate array. (Results for this 4-chip array design are described in the following Example). Alternative robust array designs also are disclosed herein.

### Example 11

### Multiplexed CF Mutation Analysis by Probe Elongation Using READ

Genomic DNA, extracted from several patients, was amplified with corresponding probes in a multiplex PCR (mPCR) reaction using the method described in L. McCurdy, Thesis, Mount Sinai School of Medicine, 2000, which is incorporated by reference. This mPCR reaction uses chimeric primers tagged with a universal sequence at the 5′ end. Antisense primers were phosphorylated at the 5′ end (Synthegen, TX). Twenty eight amplification cycles were performed using a Perkin Elmer 9600 thermal cycler, each cycle comprising a 10 second denaturation step at 94° C. with a 48 second ramp, a 10 second annealing step at 60° C. with a 36 second ramp and a 40 second extension step at 72° C. with a 38 second ramp, each reaction (50 μl) containing 500 ng genomic DNA, 1×PCR buffer (10 mM Tris HCL, 50 mM KCL, 0.1% Triton X-100), 1.5 mM MgC₁₋₂, 200 μM each of PCR grade dNTPs and 5 units Taq DNA polymerase. Optimal probe concentrations were determined for each probe pair. Following amplification, products were purified to remove all reagents using a commercially available kit (Qiagen). DNA concentration was determined by spectrophotometric analysis.

PCR products were amplified with antisense 5′-phosphorylated primers. To produce single-stranded DNA templates, PCR reaction products were incubated with 2.5 units of λ exonuclease in 1× buffer at 37° C. for 20 min, followed by enzyme inactivation by heating to 75° C. for 10 min. Under these conditions, the enzyme digests one strand of duplex DNA from the 5′-phosphorylated end and releases 5′-phosphomononucleotides (J. W. Little, et al., 1967). Single-stranded targets also can be produced by other methods known in the art.

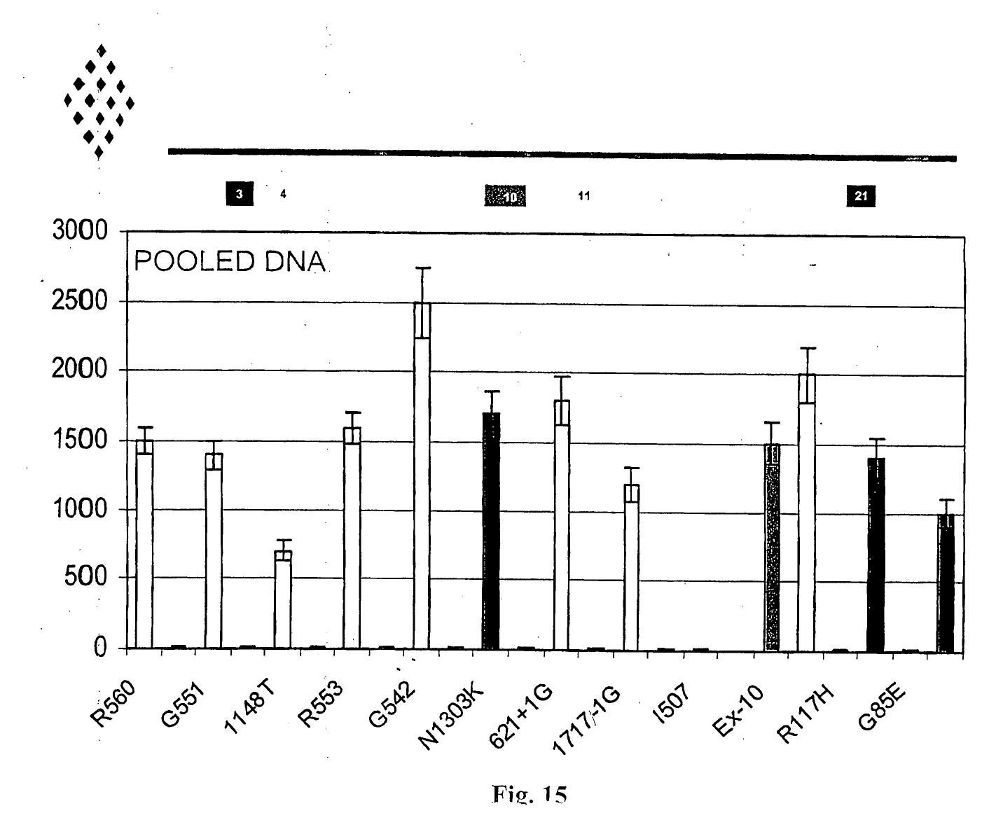

Single or pooled PCR products (20 ng each) were added to an annealing mixture containing 10 mM Tris-HCL (pH 7.4) 1 mM EDTA, 0.2 M NaCl, 0.1% Triton X-100. The annealing mixture was placed in contact with the encoded array of bead-displayed CF probes (of Example 0.10) and incubated at 37-55° C. for 20 minutes. The extension mixture—containing 3 U of Thermo Sequenase (Amersham Pharmacia Biotech NJ), X enzyme buffer with either Fluorescein-labeled or TAMRA-labeled deoxynucleotide (dNTP) analogs (NEN Life Sciences) and 1 μmole of each type of unlabeled dNTP—was then added, and the elongation reaction was allowed to proceed for 3 minutes at 60° C. The bead array was washed with deionized, sterilized water (dsH₂O) for 5-15 minutes. An image containing the fluorescence signal from each bead within the array was recorded using a fluorescence microscope equipped with a CCD camera. Images were analyzed to determine the identity of each of the elongated probes. The results are shown in FIG. 15.

### Example 12

### Use of Covering Probes

Several SNPs have been identified within exon 10 of the CFTR gene. The polymorphisms in exon 10 are listed at the end of this Example. The following nine SNPs have been identified in the sequence of Δ508, the most common mutation in the CFTR gene (Single Nucleotide Polymorphisms for Biomedical Research, SNP Consortium, Ltd. http://snp.cshl.org)
dbSNP213450 A/G
dbSNP180001 C/T
dbSNP1800093 G/T
1648 A/G
dbSNP100092 C/G
dbSNP1801178 A/G
dbSNP 1800094 A/G
dbSNP1800095 G/A

Probes are designed to accommodate all possible SNPs are synthesized and coupled to color-encoded beads. The primers for target amplification (described in Example 11) are also modified to take into account all possible SNPs. The PCR-amplified target mediates the elongation of terminally matched probes. The information collected from the analysis is twofold: identification of mutations and SNPs.

| EXON 10 POLYMORPHISMS | |
|---|---|
| (SEQ ID NO: 165) | |
| cactgtagct gtactacctt ccatctcctc aacctattcc aactatctga atcatgtgcc | 60 |
| cttctctgtg aacctctatc ataatacttg tcacactgta ttgtaattgt ctcttttact | 120 |
| ttcccttgta tcttttgtgc atagcagagt acctgaaaca ggaagtattt taaatatttt | 180 |
| gaatcaaatg agttaataga atctttacaa ataagaatat acacttctgc ttaggatgat | 240 |
| aattggaggc aagtgaatcc tgagcgtgat ttgataatga cctaataatg atgggtttta | 300 |
| tttccagact tcaCttctaa tgAtgattat gggagaactg gagccttcag agggtaaaat | 360 |
| taagcacagt ggaagaattt cattctgttc tcagttttcc tggattatgc ctggcaccat | 420 |
| taaagaaaat AtCAtctTtg gtgtttccta tgatgaatat agatacagaa gcgtcatcaa | 480 |
| agcatgccaa ctagaAgagG taagaaacta tgtgaaaact ttttgattat gcatatgaac | 540 |
| ccttcacact acccaaatta tatatttggc tccatattca atcggttagt ctacatatat | 600 |
| ttatgtttcc tctatgggta agctactgtg aatggatcaa ttaataaaac acatgaccta | 660 |
| tgctttaaga agcttgcaaa cacatgaaat aaatgcaatt tattttttaa ataatgggtt | 720 |
| catttgatca caataaatgc attttatgaa atggtgagaa ttttgttcac tcattagtga | 780 |
| gacaaacgtc tcaatggtta tttatatggc atgcatatag tgatatgtgg t | 831 |

### Example 13

### CF Mutation Analysis—on-Bead Probe Elongation with Model System

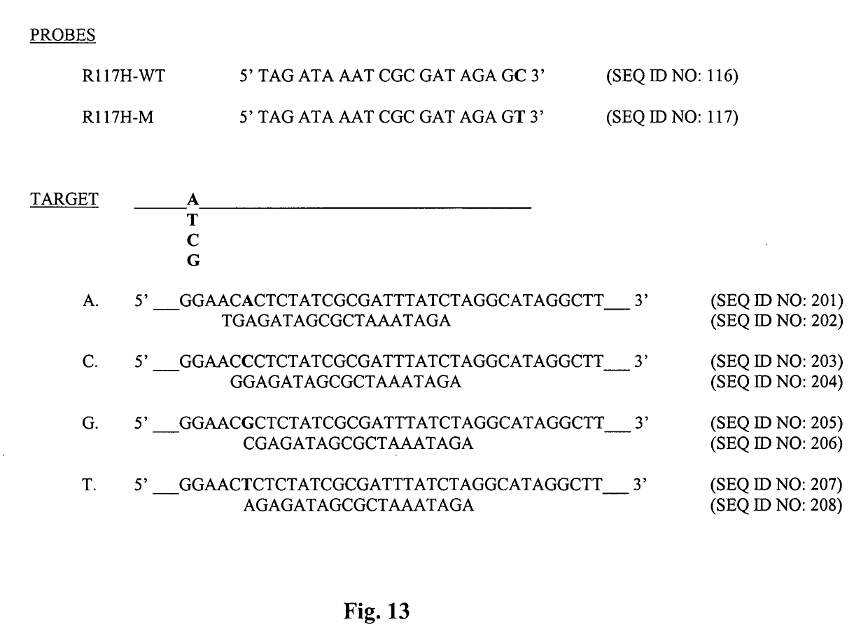

FIG. 13 provides an overview of detection of CF gene mutation R117H. The target was amplified by PCR as described in Example 11. Two 17-base probes variable at their 3′ ends were immobilized on color coded beads. The target nucleic acid sequence was added along with TAMRA-labeled dCTP, unlabeled dNTPs and thermostable DNA-polymerase.

Complementary 17-mer oligonucleotide probes variable at the 3′ end were were synthesized by a commercial vendor (Synthegen TX) to contain 5′ biotin attached by way of a 12-C spacer (Biotin-TEG) and were purified by reverse phase HPLC. Probes were immobilized on color encoded beads. Probes were attached to color-encoded beads. A synthetic 48-mer oligonucleotide also was provided to contain either A, T, C or G at a designated variable site, corresponding to a cystic fibrosis gene mutation at exon 4 (R117H).

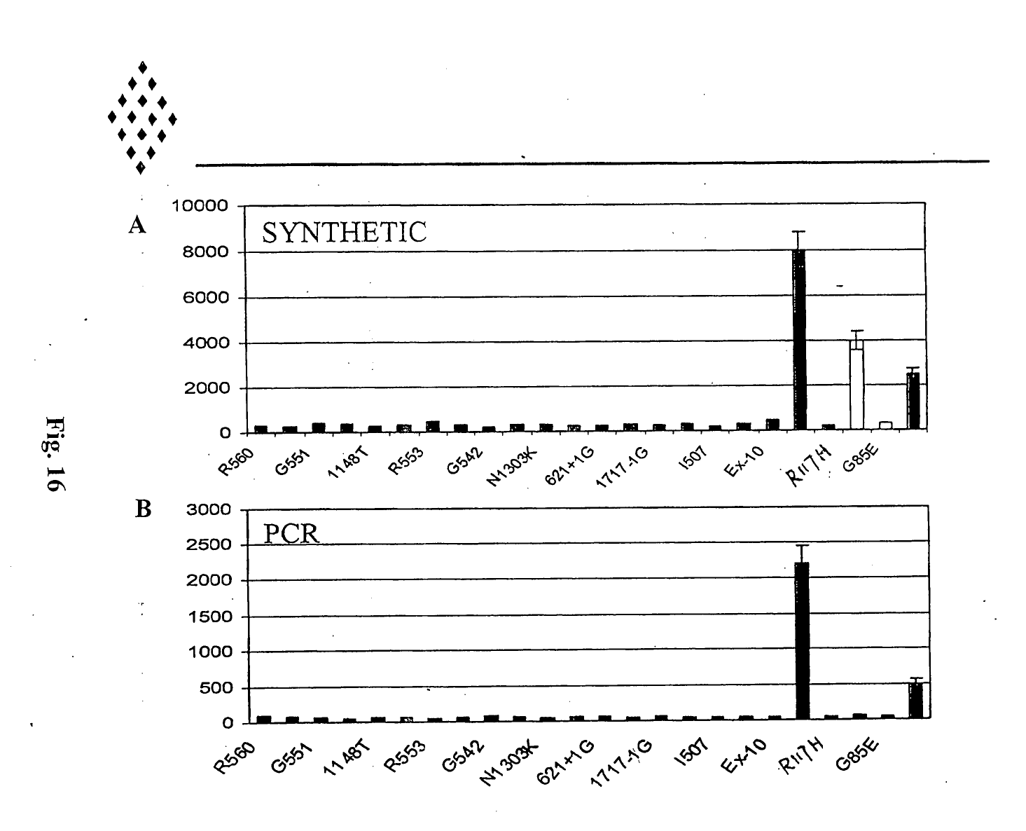

1 μM of synthetic target was added to an annealing mixture containing 10 mM Tris-HCL (pH 7.4) 1 mM EDTA, 0.2 M NaCl, 0.1% Triton X-100. The annealing mixture was placed in contact with the encoded bead array and incubated at 37° C. for 20 minutes. An elongation mixture containing 3 U of Thermo Sequenase (Amersham Pharmacia Biotech NJ), 1× enzyme buffer with TAMRA-labeled deoxynucleotide (dNTP) analogs (NEN Life Sciences) and 1 μM of each type of unlabeled dNTP was then added, and the elongation reaction was allowed to proceed for 3 minutes at 60° C. The bead array was then washed with dsH₂O for 5-15 minutes and an image containing the fluorescence signal from each bead within the array was recorded using a fluorescence microscope equipped with a CCD camera. Images were analyzed to determine the identity of each of the elongated probes. The signal was analyzed by capturing the image by a CCD camera and comparing signal intensity between two probes that can be decoded by the bead color. The wild-type probe exactly matched the added target and therefore yielded an elongation product, whereas no elongation was observed for the mutant probe. The results are shown in FIG. 16a.

### Example 14

### CF Mutation Analysis —PCR with Bead-Tagged Primers and Integrated Detection

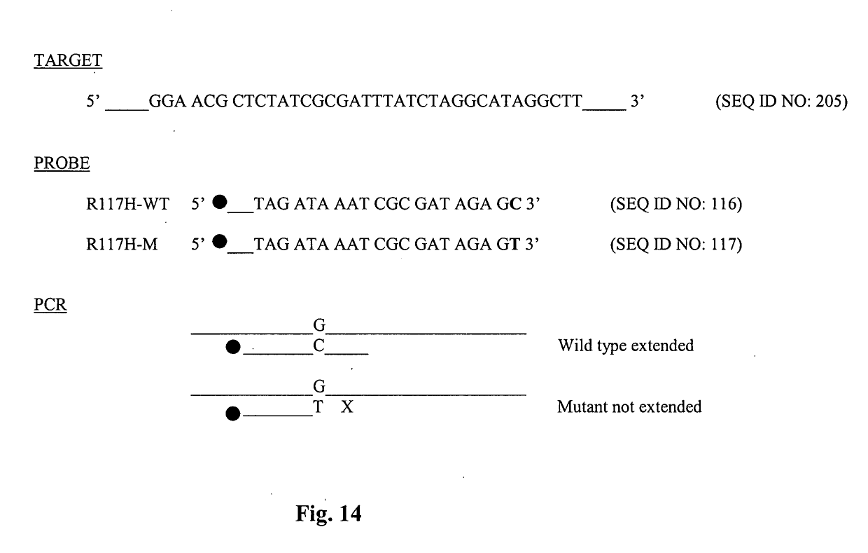

This example illustrates probe elongation on the surface of beads in suspension, followed by assembly of and immobilization of beads on the surface of a chip for image analysis. Oligonucleotides corresponding to CFTR gene mutation R117H were designed with variable 3′ ends (FIG. 14) and were synthesized to contain a 5′ biotin-TEG with a 12 C spacer (Synthegen, Texas). The probes were attached to blue stained beads as follows: 2 μM of probe were added to a bead solution in 1×TE (100 mM Tris-HCl, 10 mM EDTA), 500 mM NaCl and reacted for 45 min at room temperature. Beads were washed with 1×TE, 150 mM of NaCl for 3×, and suspended in 50 μl of the same solution. One μl of each type of bead was added to PCR mix containing 1× buffer (100 mM Tris-HCl, pH. 9.0, 1.5 mM MgCl₂, 500 mM KCl), 40 μM Cy5-labeled dCTP (Amersham Pharmacia Biotech NJ), and 80 μM of the other three types of dNTPs, and 3 U of Taq DNA polymerase (Amersham Pharmacia Biotech NJ). Wild type complementary target (40 ng) was added to the PCR mix just before amplification. Eleven cycles of PCR amplification were performed in a Perkin Elmer 9600 thermal cycler, each cycle consisting of denaturation for 30 s at 90° C., annealing for 30 s at 55° C., and elongation at 72° C. for 20 s After amplification, beads were washed four times by centrifugation in 1×TE buffer. and placed on the chip surface. Images were recorded as in previous Examples and analyzed using the software described in WO 01/98765. The results show specific amplification for beads coupled with the wild-type probe, but no amplification for beads coupled with the mutant probe. The results are shown in FIG. 16b.

This example demonstrates the integration of multiplexed PCR using bead-tagged probes with subsequent assembly of beads on planar surfaces for instant imaging analysis. In a preferred embodiment, a microfluidically connected multicompartment device may be used for template amplification as described here. For example, a plurality of compartments capable of permitting temperature cycling and housing, in each compartment, one mPCR reaction producing a subset of all desired amplicons may be used as follows: (1) perform PCR with different probe pairs in each of four compartments, using encoded bead-tagged primers as described in this Example; (2) following completion of all PCR reactions, pool the amplicon-displaying beads; (3) assemble random array; and (4) record image and analyze the data. Array assembly may be accomplished by one of several methods of the prior art including LEAPS.

### Example 15

### CF Mutation Analysis—One-Step Annealing and Elongation in Temperature-Controlled Reactor

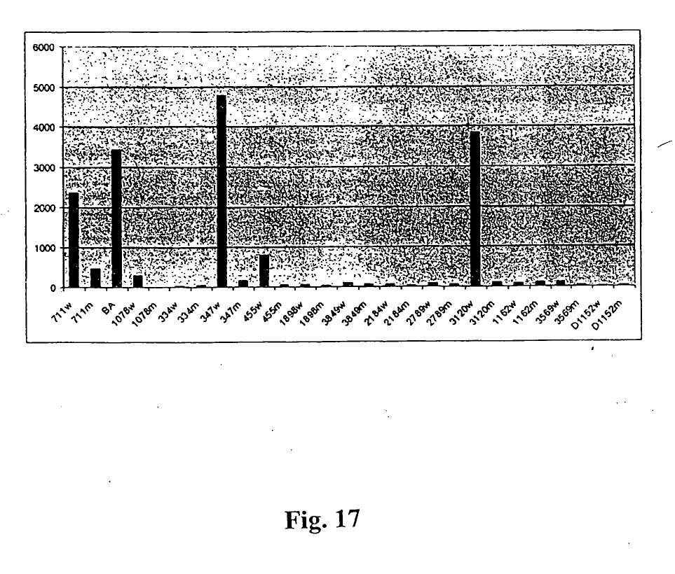

Genomic DNA, extracted from several patients, was amplified with corresponding primers in a multiplexed PCR (mPCR) reaction, as described in Example 11. Following amplification, products were purified to remove all reagents using a commercially available kit (Qiagen). DNA concentration was determined by spectrophotometric analysis. Single or pooled PCR products (20 ng each) were added to an annealing mixture containing 10 mM Tris-HCL (pH 7.4) 1 mM EDTA, 0.2 M NaCl, 0.1% Triton X-100. The annealing mixture was mixed with elongation mixture containing 3 U of Thermo Sequenase (Amersham Pharmacia Biotech, NJ), 1× enzyme buffer with either fluorescein-labeled or TAMRA-labeled deoxynucleotide (dNTP) analogs (NEN Life Sciences) and 1-10 μmole of each type of unlabeled dNTP and placed in contact with an array of oligonucleotide probes displayed on a color-encoded array. Oligonucleotides were designed and synthesized as in previous Examples. The annealing- and elongation reactions were allowed to proceed in a temperature controlled cycler. The temperature steps were as follows: three minutes each at 65° C., 60° C., 55° C., 50° C. and 45° C., with a ramp between temperatures of less than 30 seconds. The bead array was then washed with dsH₂O for 5 to 15 min. and an image containing the fluorescence signal from each bead within the array was recorded using a fluorescence microscope equipped with a CCD camera. Images were analyzed to determine the identity of each of the elongated probes. Typical results are shown in FIG. 17.

### Example 16

### Pooling of Covering Probes

To analyze designated polymorphisms, 20-mer oligonucleotide elongation probes of 30-50% G+C base composition were designed to contain a variable site (G/T) at the 3′ end, to be aligned with the designated polymorphic site. Two non-designated polymorphic sites were anticipated at position 10 (C/A) and at 15 (T/G). A summary of the design follows:

Wild-type probe sequence:

| | |
|---|---|
| Oligo 1: | “G” at position 20, “C” at 10, and “T” at 15. |
| Oligo 2: | “G” at position 20, “C” at 10, and “G” at 15. |
| Oligo 3: | “G” at position 20, “A” at 10, and “T” at 15. |
| Oligo 4: | “G” at position 20, “A” at 10, and “G” at 15. |

Mutant Probe Sequence:

| | |
|---|---|
| Oligo 1: | “T” at position 20, “C” at 10, and “T” at 15. |
| Oligo 2: | “T” at position 20, “C” at 10, and “G” at 15. |
| Oligo 3: | “T” at position 20, “A” at 10, and “T” at 15. |
| Oligo 4: | “T” at position 20, “A” at 10, and “G” at 15. |

All of the probes were pooled and attached to a single type of color-coded bead using protocols of previous Examples. When single-stranded target is added to these beads displaying pooled probes, one of the probes will yield elongation product as long as it is perfectly aligned with the designated polymorphism.

### Example 17

### Designated Polymorphisms in Heterozygous and Homozygous Configurations

To distinguish between heterozygous and homozygous configurations, the design of the previous Example is augmented to contain a second set of probes to permit the identification of the C/A designated polymorphism aligned with the probes' 3′ ends, and to permit calling of heterozygous versus homozygous mutations.

As in the previous example, two non-designated polymorphic sites are anticipated at positions 10 (C/A) and 15 (T/G). A summary of the design follows:

| | |
|---|---|
| Set #1: | |
| Oligo 1: | “C” at position 20, “C” at 10, and “T” at 15. |
| Oligo 2: | “C” at position 20, “C” at 10, and “G” at 15. |
| Oligo 3: | “C” at position 20, “A” at 10, and “T” at 15. |
| Oligo 4: | “C” at position 20, “A” at 10, and “G” at 15. |
| Set #2: | |
| Oligo 5: | “A” at position 20, “C” at 10, and “T” at 15. |
| Oligo 6: | “A” at position 20, “C” at 10, and “G” at 15. |
| Oligo 7: | “A” at position 20, “A” at 10, and “T” at 15. |
| Oligo 8: | “A” at position 20, “A” at 10, and “G” at 15. |

Oligonucleotides from set #1 are pooled and attached to a single type of color (e.g. green) coded bead using protocols of previous Examples. Oligonucleotides from set # 2 were pooled and attached to a second type of color (e.g. orange) coded bead using protocols of previous Examples. Beads were pooled and immobilized on the surface of chip as described earlier. Next, target was introduced, and on-chip reactions performed as described in previous Examples. If probes on green beads only are elongated, the individual has a normal (or wild-type) allele. If probes on orange beads only are elongated, the individual is homozygous for the mutation. I If probes on green as well as origan beads are elongated, the individual is heterozygous for that allele. This design is useful for the identification of known and unknown mutations.

### Example 18

### Confirmatory Sequencing (“Resequencing”)

The design of the present invention can be used for re-sequencing of a specific area. This test can be used when on-chip probe elongation reaction requires confirmation, as in the case of reflex tests for 1506V, 1507V, F508C and 7T in the CF mutation panel. The sequence in question, here 20 bases to 30 bases in length, is sequenced on-chip by multiplexed interrogation of all variable sites. This is accomplished by designing specific probes for ambiguous locations, and by probe-pooling as described in Examples 16 and 17.

### Example 19

### Elongation with One Labeled dNTP and Three Unlabeled dNTPs

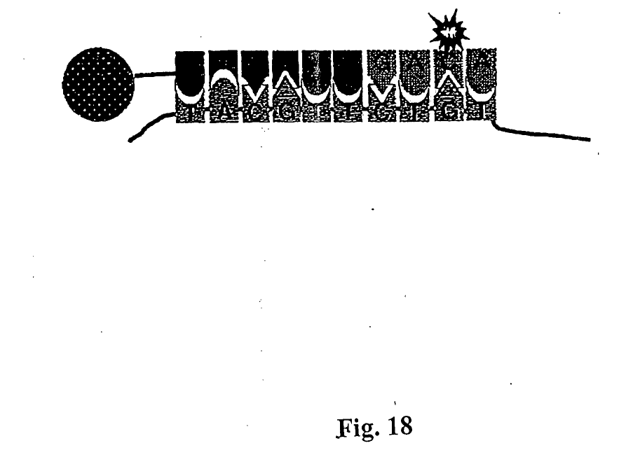

By way of incorporating at least one labeled dNTP, all elongation products are detected in real-time and identified by their association with coded solid phase carriers. Using assay conditions described in connection with Examples 6 and 7, tetramethylrhodamine-6-dCTP and unlabeled dATP, dTTP and dGTP were provided in an elongation reaction to produce a fluorescently labeled elongation product as illustrated FIG. 18. Other dye labeling of dNTPs (as in BODIPY-labeled dUTP and Cy5-labeled dUTP) may be used. Similarly, any other labeled dNTP can be used. The length of the elongation product depends on the amount of labeled dNTP tolerated by the DNA polymerase. Available enzymes generally exhibit a higher tolerance for strand-modifying moieties such as biotin and digoxigenin which may then be reacted in a second step with labeled avidins or antibodies to accomplish indirect labeling of elongation products. When using these small molecules, elongation products measuring several hundred bases in length are produced.

### Example 20

### Extension with One Labeled ddNTP, Three Unlabeled dNTPs

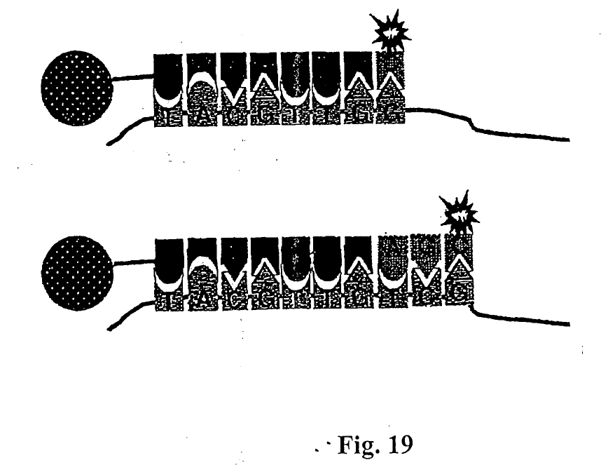

TAMRA-labeled ddCTP may be incorporated to terminate the extension reaction, as illustrated in FIG. 19. On-chip reactions using TAMRA-labeled ddCTP were performed as described in Examples 6 and 7. In a reaction mixture containing TAMRA-ddCTP and unlabeled dTTP, dATP and dGTP, following annealing of the target to the matching probe, the extension reaction terminates when it completes the incorporation of the first ddCTP. This may occur with the very first base incorporated, producing a single base extension product, or it may occur after a number of unlabeled dNTPs have been incorporated.

### Example 21

### Elongation with Four Unlabeled dNTPs, Detection by Hybridization of Labeled Probe

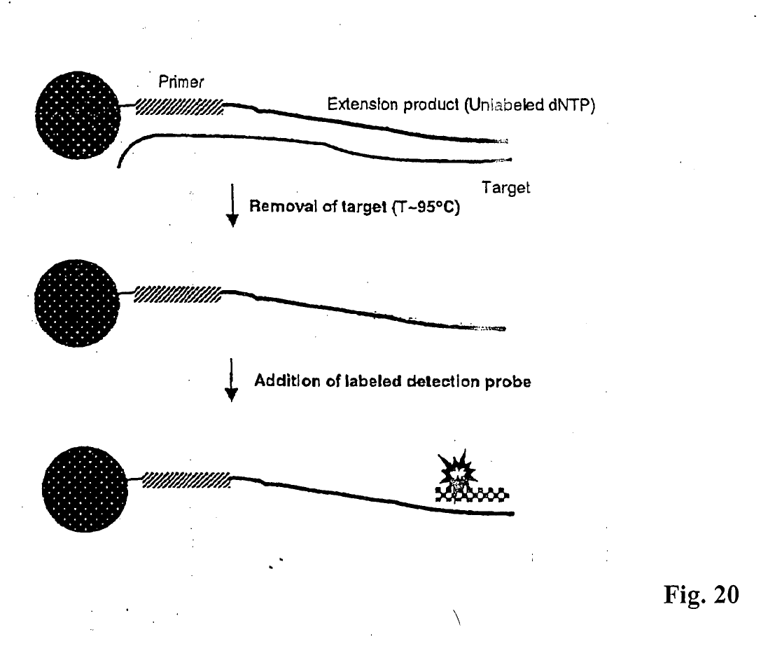

Probes are elongated using a full set of four types of unlabeled dNTPs, producing, under these “native” conditions for the polymerase, elongation products measuring several hundred bases in length, limited only by the length of the annealed template and on-chip reaction conditions. The clongation product is detected, following denaturation at high temperature, in a second step by hybridization with a labeled oligonucleotide probe whose sequence is designed to be complementary to a portion of the elongation product This process is illustrated in FIG. 20.

### Example 22

### Elongation with Four Unlabeled dNTPs, Detection via Labeled Template

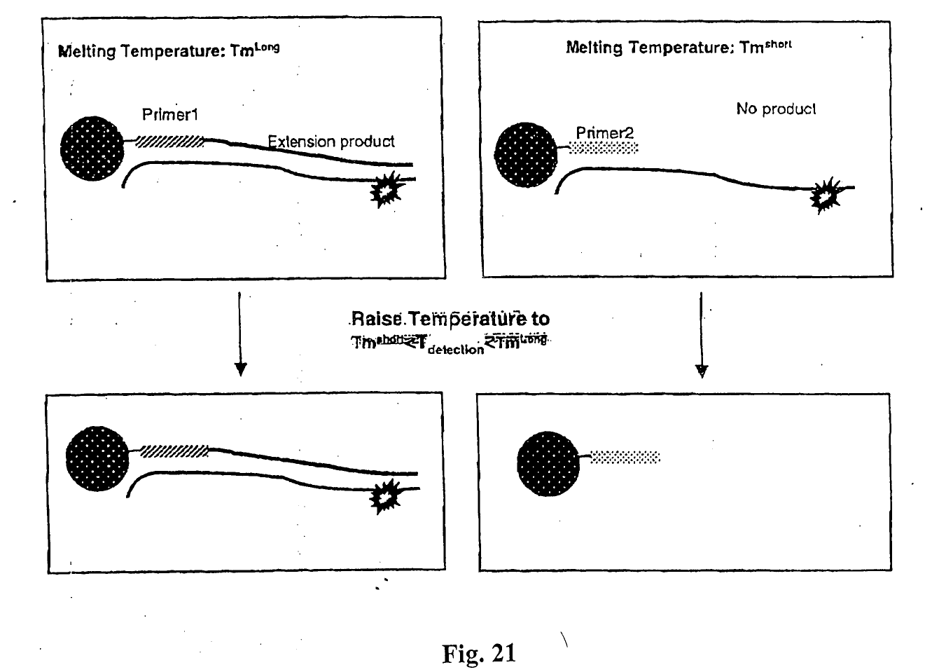

As with standard protocols in routine use in multiplexed hybridization assays, the DNA target to be analyzed can itself be labeled in the course of PCR by incorporation of labeled probes. Under conditions such as those described in Examples 6 and 7, a labeled target is annealed to probes. Matching probes are elongated using unlabeled dNTPs. Following completion of the elongation reaction, detection is performed by setting the temperature (T_{det}) to a value above the melting temperature (T_{non-match}) of the complex formed by target and non-matched probe, but below the melting temperature (T_{match}) of the complex formed by target and matched, and hence elongated, probe. The latter complex, displaying a long stretch of duplex region, will be significantly more stable than the former so that (T_{non-match})<T<(T_{match}). Typical values for T are in the range of 70° C. to 80° C. Under these conditions, only the complex formed by target and elongated probe will stable, while the complex formed by target and non-matching probe, and hence the fluorescence signal from the corresponding solid phase carrier, will be lost. That is, in contrast to other designs, it is the decrease of signal intensity associated with the non-matching probe which is detected, rather than the increase in intensity associated the matching probe. FIG. 21 illustrates the design which eliminates the need for labeled dNTPs or ddNTPs. This is useful in the preferred embodiments of this invention, where labeled dNTPs or ddNTPs can absorb non-specifically to encoded particles, thereby increasing the background of the signal and decreasing the discriminatory power of the assays. In addition, by using a labeled target, this protocol is directly compatible with methods of polymorphism analysis by hybridization of sequence-specific oligonucleotides.

### Example 23

### Real-Time on-Chip Signal Amplification

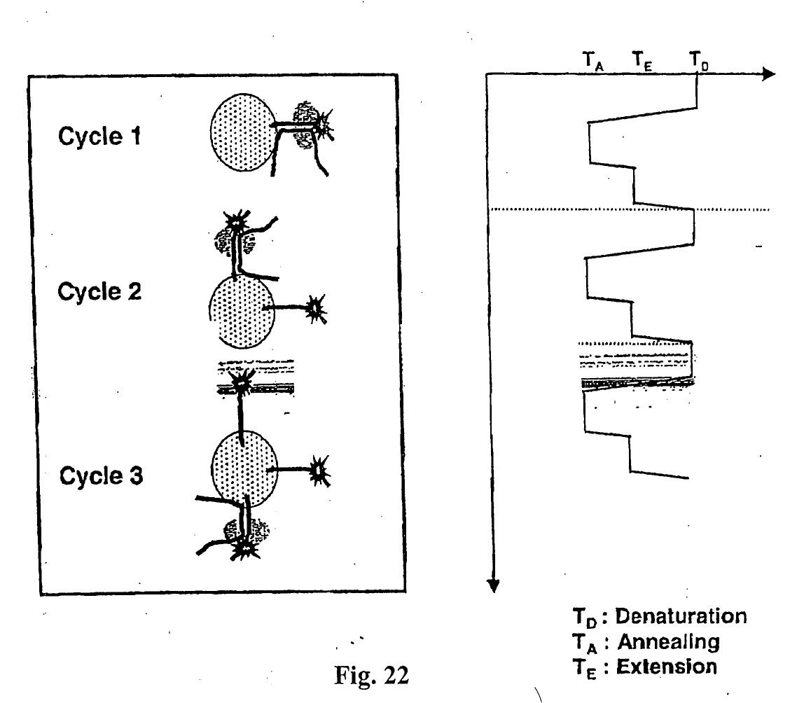

A standard temperature control apparatus used with a planar geometry such as that illustrated in FIG. 22 permits the application of programmed temperature profiles to a multiplexed-extension of SSPs. Under conditions of Examples 6 and 7, a given template mediates the elongation of one probe in each of multiple repeated “denature-anneal-extend” cycles. In the first cycle, a target molecule binds to a probe and the probe is elongated or extended. In the next cycle, the target molecule disassociates from the first probe in the “denature” phase (at a typical temperature of 95° C.), then anneals with another probe molecule in the “anneal” phase (at a typical temperature of 55° C.) and mediates the extension of the probe in the “extend” phase (at a typical temperature of 72° C.). In N cycles, each template mediates the extension of N probes, a protocol corresponding to linear amplification (FIG. 30). In a preferred embodiment of this invention, in which planar arrays of encoded beads are used to display probes in a multiplexed extension reaction, a series of temperature cycles is applied to the reaction mixture contained between two planar, parallel substrates. One substrate permits direct optical access and direct imaging of an entire array of encoded beads. The preferred embodiment provides for real-time amplification by permitting images of the entire bead array to be recorded instantly at the completion of each cycle.

Genomic, mitochondrial or other enriched DNA can be used for direct detection using on-chip linear amplification without sequence specific amplification. This is possible when an amount of DNA sufficient for detection is provided in the sample. In the bead array format, if 104 fluorophores are required for detection of signal from each bead, 30 cycles of linear amplification will reduce the requisite number to ˜300. Assuming the use of 100 beads of the requisite type within the array, the requisite total number of fluorophores would be ˜10⁵, a number typically available in clinical samples. For example, typical PCR reactions for clinical molecular typing of HLA are performed with 0.1 to 1 μg of genomic DNA. One μg of human genomic DNA corresponds to approximately 10⁻¹⁸ moles, thus, 6×10⁵ copies of the gene of interest This small amount of sample required by the miniaturized bead array platform and on-chip amplification makes the direct use of pre-PCR samples possible. This not only simplifies sample preparation but, more importantly, eliminates the complexity of multiplexed PCR, frequently a rate limiting step in the development of multiplexed genetic analysis.

### Example 24

### Construction of a Probe Library for Designated and Unselected Polymorphisms for CF Mutation Analysis

To increase the specificity of elongation probes and avoid false positives; elongation probes were designed to accommodate all known polymorphisms present in a target sequence. In addition, PCR primers were designed taking into consideration designated and non-designated polymorphisms.

The G/C mutation at position 1172-[[.]] of R347P on Exon 7 within the CFTR gene, one of 25 mutations within the standard population carrier screening panel for cystic fibrosis, was selected as a designated polymorphism. There are 3 CF mutations within Exon 7 included in the mutation panel for general population carrier screening (American College of Medical Genetics http://www.faseb.org/genetics/acmg). A polymorphism G/T/A at the same site has been reported (Cystic Fibrosis Mutation Database http://www.genet.sickkids.on.ca/cftr), and in addition, non-designated polymorphisms have been reported at positions 1175, 1178, 1186, 1187 and 1189. All of these polymorphisms can interfere with desired probe elongation.

The construction of a set of degenerate probes for eMAP is illustrated below for R347P (indicated by the bold-faced G) which is surrounded by numerous non-designated polymorphisms, indicated by capital letters:

| | | |
|---|---|---|
| | 5′ 3′ | |
| Normal Target Sequence for Elongation: | Gca Tgg Cgg tca ctC GgG a | (SEQ ID NO: 166) |
| Degenerate Elongation Probe Set: | Ngt Ycc Ycc agt gaY RcY t | (SEQ ID NO: 167) |
| | 3′ 5′ | |

where N=a, c, g or t; R (puRines)=a or g and Y (pyrimidines)=c or t, implying a degeneracy of 128 for the set.

Primer Pooling for Mutation Analysis—The principal objective in the construction of a degenerate set is to provide at least one probe sequence to match the target sequence sufficiently closely to ensure probe annealing and elongation. While this is always attainable in principle by providing the entire set of possible probe sequences associated with the designated polymorphism, as in the preferred mode of constructing covering sets, the degree of degeneracy of that set, 128 in the example, would lead to a corresponding reduction in assay signal intensity by two orders of magnitude if all probes were to be placed onto a single bead type for complete probe pooling. Splitting pools would improve the situation by distributing the probe set over multiple bead types, but only at the expense of increasing array complexity.

First, the probe pool was split into a minimum of two or more pools, each pool providing the complementary composition, at probe position M (i.e., the probe's 3′ terminus), for each of the possible compositions of the designated polymorphic site. In the example, four such pools are required for a positive identification of the designated target composition. Next, non-designated polymorphic sites were examined successively in the order of distance from the designated site. Among these, positions within the TEI region are of special importance to ensure elongation. That is, each pool is constructed to contain all possible probe compositions for those non-designated sites that fall within the TEI region. Finally, as with the construction of degenerate probes for cloning and sequencing of variable genes, the degeneracy of the set is minimized by placing neutral bases such as inosine into those probe positions which are located outside the TEI region provided these are known never to be juxtaposed to G in the target. In the example, non-designated polymorphisms in probe positions M-16 and M-18 qualify. That is, the minimal degeneracy of each of the four pools would increase to four, producing a corresponding reduction in signal intensity. As an empirical guideline, signal reduction preferably will be limited to a factor of eight.

In total, four pools, each uniquely assigned to one bead type and containing eight degenerate probe sequences, will cover the target sequence. These sequences are analogous to those shown below for pools variable at M:

Probe pool for CF mutation R347P

| | | |
|---|---|---|
| R347P | Cgt Acc Gcc agt gaG GgC | (SEQ ID NO: 168) |
| | 3′ 5′ | |
| POOL 1 | Cgt Acc Gcc agt gaG IgI | (SEQ ID NO: 169) |
| | Cgt Acc Gcc agt gaC IgI | (SEQ ID NO: 170) |
| | Cgt Acc Ccc agt gaG IgI | (SEQ ID NO: 171) |
| | Cgt Acc Ccc agt gaC IgI | (SEQ ID NO: 172) |
| | Cgt Tcc Gcc agt gaG IgI | (SEQ ID NO: 173) |
| | Cgt Tcc Gcc agt gaC IgI | (SEQ ID NO: 174) |
| | Cgt Tcc Ccc agt gaG IgI | (SEQ ID NO: 175) |
| | Cgt Tcc Ccc agt gaC IgI | (SEQ ID NO: 176) |
| POOL 2 | Ggt Acc Gcc agt gaG IgI | (SEQ ID NO: 177) |
| | Ggt Acc Gcc agt gaC IgI | (SEQ ID NO: 178) |
| | Ggt Acc Ccc agt gaG IgI | (SEQ ID NO: 179) |
| | Ggt Acc Ccc agt gaC IgI | (SEQ ID NO: 180) |
| | Ggt Tcc Gcc agt gaG IgI | (SEQ ID NO: 181) |
| | Ggt Tcc Gcc agt gaC IgI | (SEQ ID NO: 182) |
| | Ggt Tcc Ccc agt gaG IgI | (SEQ ID NO: 183) |
| | Ggt Tcc Ccc agt gaC IgI | (SEQ ID NO: 184) |
| POOL 3 | Agt Acc Gcc agt gaG IgI | (SEQ ID NO: 185) |
| | Agt Acc Gcc agt gaC IgI | (SEQ ID NO: 186) |
| | Agt Acc Ccc agt gaG IgI | (SEQ ID NO: 187) |
| | Agt Acc Ccc agt gaC IgI | (SEQ ID NO: 188) |
| | Agt Tcc Gcc agt gaG IgI | (SEQ ID NO: 189) |
| | Agt Tcc Gcc agt gaC IgI | (SEQ ID NO: 190) |
| | Agt Tcc Ccc agt gaG IgI | (SEQ ID NO: 191) |
| | Agt Tcc Ccc agt gaC IgI | (SEQ ID NO: 192) |
| POOL 4 | Tgt Acc Gcc agt gaG IgI | (SEQ ID NO: 193) |
| | Tgt Acc Gcc agt gaC IgI | (SEQ ID NO: 194) |
| | Tgt Acc Ccc agt gaG IgI | (SEQ ID NO: 195) |
| | Tgt Acc Ccc agt gaC IgI | (SEQ ID NO: 196) |
| | Tgt Tcc Gcc agt gaG IgI | (SEQ ID NO: 197) |
| | Tgt Tcc Gcc agt gaC IgI | (SEQ ID NO: 198) |
| | Tgt Tcc Ccc agt gaG IgI | (SEQ ID NO: 199) |
| | Tgt Tcc Ccc agt gaC IgI | (SEQ ID NO: 200) |

In general, the type of non-designated polymorphisms on the antisense strand may differ from that on the sense strand, and it may then be advantageous to construct degenerate probe sets for the antisense strand. As with the construction of degenerate elongation probes, degenerate hybridization probe sets may be constructed by analogous rules to minimize the degeneracy.

### Example 25

### “Single Tube” CF Mutation Analysis by eMAP

This example is concerned with methods and compositions for performing an eMAP assay, wherein the annealing and elongation steps occur in the reactor. This embodiment is useful because it obviates the need for sample transfer between reactors as well as purification or extraction procedures, thus simplifying the assay and reducing the possibility of error. A non-limiting exemplary protocol follows.

Genomic DNA extracted from several patients was amplified with corresponding primers in a multiplex PCR (mPCR) reaction. The PCR conditions and reagent compositions were as follows.

PRIMER DESIGN: Sense primers were synthesized without any modification and antisense primers with “Phosphate” at the 5′ end. Multiplex PCR was performed in two groups. Group one amplification includes exon 5, 7, 9, 12, 13, 14B, 16, 18 and 19. Amplifications for group 2 includes primers for exon 3, 4, 10, 11, 20, 21 and intron 19. The 5′ phosphate group modification on exon 5, 7, and 11 was included on forward primer to use antisense target for probe elongation. While sense target was used for all other amplicons by placing phosphate group on reverse primer.

### PCR Master Mix Composition

For 10 ul reaction/sample:

| Components | Volume (μl) |
|---|---|
| 10X PCR buffer | 1.0 |
| 25 mM MgCl₂ | 0.7 |
| dNTPs (2.5 mM) | 2.0 |
| Primer mix (Multiplex 10x) | 1.5 |
| Taq DNA polymerase | 0.3 |
| ddH2O | 1.5 |
| DNA | 3.0 |
| Total | 10 |

PCR Cycling
94° C. 5 min, 94° C. 10 sec., 60° C. 10 sec., 72° C. 40 sec
72° C. 5 min., Number of cycles: 28-35

The reaction volume can be adjusted according to experimental need. Amplifications are performed using a Perkin Elmer 9600 thermal cycler. Optimal primer concentrations were determined for each primer pair. Following amplifications, 5 ul of the product was removed for gel electrophoresis. Single stranded DNA targets were generated as follows: Two microliters of exonuclease was added to 5 pt of PCR product, incubated at 37° C. for 15 minutes and enzyme was denatured at 80° C. for 15 minutes. After denaturation, 1 μl of 10× exonuclease buffer was added with 1 μl of λ exonuclease (5 U/μl) and incubated at 37° C. for 20 minutes and the reaction was stopped by heating at 75° C. for 10 minutes.

### On Chip Elongation

Wild type and mutant probes for 26 CF mutations were coupled on the bead surface and assembled on the chip array. The probes were also divided into two groups. A third group was assembled for reflex test including 5T/7T/9T polymorphisms.

Elongation Group 1, total 31 groups on the chip surface.

| Bead cluster # | Mutation |
|---|---|
| 1 | G85E-WT |
| 2 | G85E-M |
| 3 | 621 + 1G > T-WT |
| 4 | 621 + 1G > T-M |
| 5 | R117H-WT |
| 6 | R117H-M |
| 7 | β Actin |
| 8 | I148T-WT |
| 9 | I148T-M |
| 10 | 508-WT |
| 11 | F508 |
| 12 | I507 |
| 13 | G542X-WT |
| 14 | G542X-M |
| 15 | G551D-WT |
| 16 | G551D-M |
| 17 | R553X-WT |
| 18 | R553X-M |
| 19 | BIOTIN |
| 20 | 1717 − 1G > A-WT |
| 21 | 1717 − 1G > A-M |
| 22 | R560T-WT |
| 23 | R560T-M |
| 24 | 3849 + 10kbT-WT |
| 25 | 3849 + 10kbT-M |
| 26 | W1282X-WT |
| 27 | W1282X-M |
| 28 | N1303K-WT |
| 29 | N1303K-M |
| 30 | OLIGO-C |

| | |
|---|---|
| Cluster # | Mutation |

Elongation Group 2, total 28 groups on the chip surface.

| | |
|---|---|
| 1 | 711 + 1G > T-WT |
| 2 | 711 + 1G > T-M |
| 3 | R334W-WT |
| 4 | R334W-M |
| 5 | 1078delT-WT |
| 6 | 1078delT-M |
| 7 | β Actin |
| 8 | R347P-WT |
| 9 | R347P-M |
| 10 | A455E-WT |
| 11 | A455E-M |
| 12 | 1898 + 1G > A-WT |
| 13 | 1898 + 1G > A-WT |
| 14 | 2184delA-WT |
| 15 | 2184delA-M |
| 16 | 2789 + 5G-WT |
| 17 | 2789 + 5G-M |
| 18 | BIOTIN |
| 19 | 3120 + 1G > A-WT |
| 20 | 3120 + 1G > A-WT |
| 21 | R1162X-WT |
| 22 | R1162X-M |
| 23 | 3659delC-WT |
| 24 | 3659delC-M |
| 25 | D1152-WT |
| 26 | D1152-M |
| 27 | OLIGO-C |

mPCR group 2:
Elongation Group 3, total 6 groups

| | |
|---|---|
| 1 | β Actin |
| 1 | Oligo C |
| 2 | 5T |
| 3 | 7T |
| 4 | 9T |
| 5 | Biotin |

Elongation reaction buffer has been optimized for use in uniplex and/or multiplex target elongation assays and composed of, Tris-HCL (pH 8.5) 1.2 mM, EDTA 1 μM, DTT 10 μM, KCl 1 μM, MgCl₂ 13 μM, 2-Mercaptoethanol 10 μM, Glycerol 0.5%, Tween-20 0.05%, and Nonidet 0.05%. Ten microliters of elongation reaction mixture was added on each chip containing 1× Reaction buffer 0.1 μM of Labeled dNTP, 1.0 μM of dNTPs mix, 3 U of DNA polymerase and 5 μl (˜5 ng) of target DNA (patient sample). The reaction mix was added on the chip surface and incubated at 53° C. for 15 min and then at 60° C. for 3 min. The chip was washed with wash buffer containing 0.01% SDS, covered with a clean cover slip and analyzed using a Bioarray Solutions imaging system. Images are analyzed to determine the identity of each of the elongated probes.

### Example 26

### CF Mutation Analysis—Single Tube Single Chip-One Step Elongation

Probes for 26 CF mutations and controls were coupled on the surface of 51 types of beads. Probe coupled beads were assembled on the surface of a single chip. Genomic DNA was extracted from several patients and was amplified with corresponding primers in a multiplexed PCR (mPCR) reaction, as described in the previous example. Following amplification, single stranded DNA products were produced using λ exonuclease. Single or pooled PCR products (˜5 ng) were added to a reaction mixture containing reaction buffer, deoxynucleotide (dNTP) analogs (NEN Life Sciences), each type of unlabeled dNTP, and DNA polymerase (Amersham Pharmacia Biotech, NJ). The annealing/elongation reaction was allowed to proceed in a temperature controlled cycler. The temperature steps were as follows: 20 minutes at 53° C., and 3 minutes at 60° C. The bead array was then washed with dsH₂O containing 0.01% SDS for 5 to 15 minutes. An image containing the fluorescent signal form each bead within the array was recorded using a fluorescence microscope and a CCD camera. Images were analyzed to determine the identity of each of the elongated probes.

The composition of bead chip containing 26 CF mutations is provided below.

Elongation Group 4, total 51 groups

| Cluster # | Mutation |
|---|---|
| 1 | β Actin |
| 2 | G85E-WT |
| 3 | G85E-M |
| 4 | 621 + 1G > T-WT |
| 5 | 621 + 1G > T-M |
| 6 | R117H-WT |
| 7 | R117H-M |
| 8 | I148T-WT |
| 9 | I148T-M |
| 10 | 711 + 1G > T-WT |
| 11 | 711 + 1G > T-M |
| 12 | A455E-WT |
| 13 | A455E-M |
| 14 | 508-WT |
| 15 | F508 |
| 16 | I507 |
| 17 | R533-WT |
| 18 | R533-M |
| 19 | G542-WT |
| 20 | G542-M |
| 21 | G551D-WT |
| 22 | G551D-M |
| 23 | R560-WT |
| 24 | R560-M |
| 25 | 1898 + 1G-WT |
| 26 | 1898 + 1G-M |
| 27 | 2184delA-WT |
| 28 | 2184delA-M |
| 29 | 2789 + 5G > A-WT |
| 30 | 2789 + 5G > A-M |
| 31 | 3120 + 1G-WT |
| 32 | 3120 + 1G-WT |
| 33 | D1152-WT |
| 34 | D1152-M |
| 35 | R1162-WT |
| 36 | R1162-M |
| 37 | OLIGO-C |
| 38 | W1282X-WT |
| 39 | W1282-M |
| 40 | N1303K-WT |
| 41 | N1303-M |
| 42 | R334-WT |
| 43 | R334-M |
| 44 | 1078delT-WT |
| 45 | 1078delT-M |
| 46 | 3849 − 10kb-WT |
| 47 | 3849 − 10kb-M |
| 49 | 1717 − 1G > A-WT |
| 50 | 1717 − 1G > A-WT |
| 51 | Biotin |

### Example 27

### Identification of Three or More Base Deletions and/or Insertions by eMAP

Elongation was used to analyze mutations with more than 3 base deletions or insertions. Probes were designed by placing mutant bases 3-5 base before 3′ end. The wild type probes were designed to either include or exclude mutant bases (terminating before mutations). The following is an example of mutations caused by a deletion of ATCTC and/or insertion of AGGTA. The probe designs are as follows:

| | |
|---|---|
| 1. | WT1- ------------------------ATCTCgca |
| 2. | WT2- ------------------------ |
| 3. | M1 ------------------------gca (deletion only) |
| 4. | M2 ------------------------AGGTAgca (deletion |
| | and insertion) |

Wild type probes were either coupled on the surface of differentially encoded beads or pooled as described in this invention. Probes for mutation 1 (M1: deletion) and 2 (M2: insertion) were coupled on different beads. Both wild type probes provide similar information, while the mutant probes can show the type of mutation identified in a specific sample.

### Example 28

### Hairpin Probes

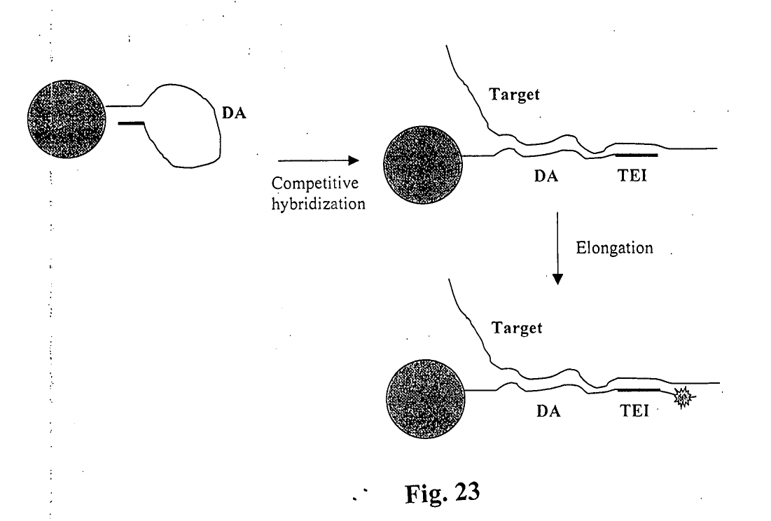

In certain embodiments of this invention, bead-displayed priming probes form hairpin structures. A hairpin structure may include a sequence fragment at the 5′ end that is complementary to the TEI region and the DA sequence, as shown in FIG. 23. During a competitive hybridization reaction, the hairpin structure opens whenever the DA region preferentially hybridizes with the target sequence. Under this condition, the TEI region will align with the designated polymorphic site and the elongation reaction will occur. The competitive nature of the reaction can be used to control tolerance level of probes.

### Example 29 Analysis of Cystic Fibrosis and Ashkenazi Jewish Disease Mutations by Multiplexed Elongation of Allele Specific Oligonucleotides Displayed on Custom Bead Arrays

A novel assay for the high throughput multiplexed analysis of mutations has been evaluated for ACMG+ panel of Cystic Fibrosis mutations. In addition, an Ashkenazi Jewish disease panel also has been developed to detect common mutations known to cause Tay-Sachs, Canavan, Gaucher, Niemann-Pick, Bloom Syndrome, Fancomi Anemia, Familial Dysautonomia, and mucolipodosis IV.

In elongated-mediated multiplexed analysis of polymorphisms (eMAP), allele specific oligonucleotides (ASO) containing variable 3′ terminal sequences are attached to color-encoded beads which are in turn arrayed on silicon chips. Elongation products for normal and mutant sequences are simultaneously detected by instant imaging of fluorescence signals from the entire array.

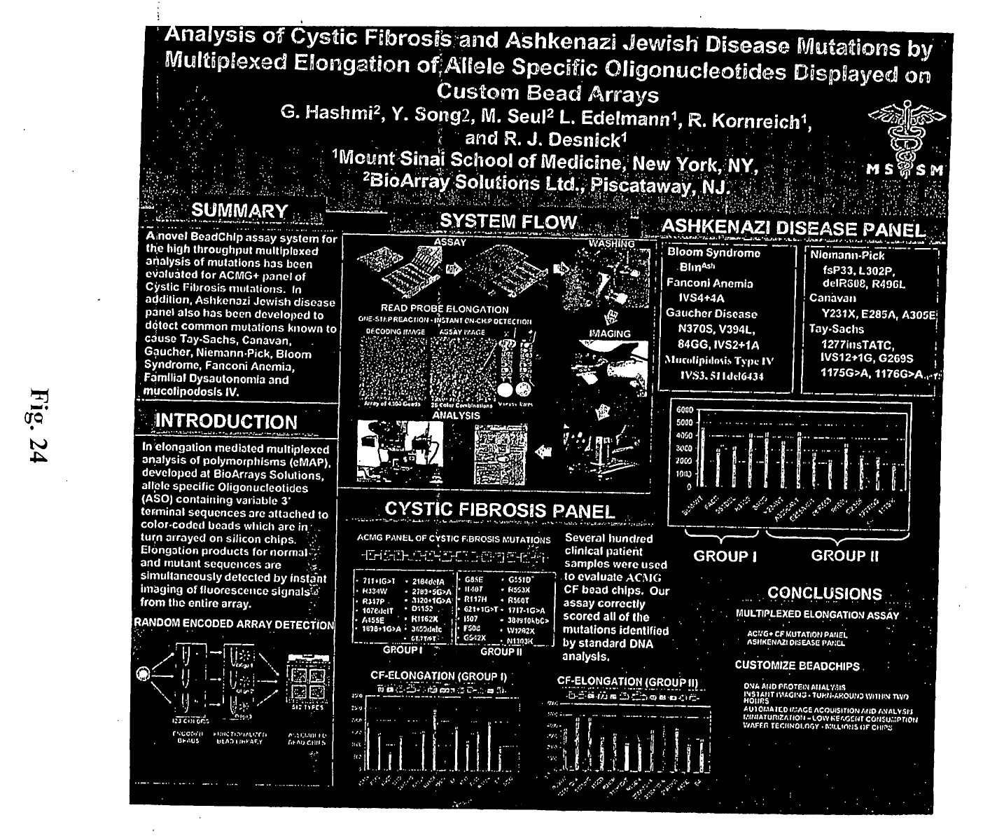

In this example, several hundred clinical patient samples were used to evaluate ACMG CF bead chips. As shown in FIG. 24, the assay correctly scored all of the mutations identified by standard DNA analysis.

In summary, a multiplexed elongation assay comprising customized beads was used to study mutations corresponding to ACMG+ and Ashkenazi disease panels. The customized beads can be used for DNA and protein analysis. The use of these customized beads are advantageous for several reasons including (1) instant imaging—the turnaround time for the assay is within two hours (2) automated image acquisition and analysis (3) miniaturization, which means low reagent consumption, and (4) the bead chips are synthesized using wafer technology, so that millions of chips can be mass-produced, if desired.

1-69. (canceled)

70. A method of identifying one or more nucleotides at each of two or more designated sites in one or more targets, the method comprising the following steps:
a) providing a set of oligonucleotide primer pairs, each pair capable of annealing with complementary polynucleotide strands to delineate a region of the corresponding target which includes a designated polymorphic site;
b) contacting said set of oligonucleotide primers with said targets under conditions allowing formation of pairs of complementary amplicon strands including, designated polymorphic sites corresponding to designated polymorphic sites in corresponding targets;
c) selecting a set of encoded probes wherein differently encoded probes have different nucleotide sequences, selected such that different probes are differently encoded and probes of a first type are complementary, in whole or in substantial part, to a subsequence of an amplicon sense strand, where said amplicon sense strand is in molar excess over its complementary antisense amplicon strand;
and encoded probes of at least a second type are complementary, in whole or in substantial part, to a subsequence of an amplicon antisense strand, where the amplicon antisense strand is provided in molar excess over its complementary sense amplicon strand;
d) associating said set of probes with a set of encoded carriers, such that the encoding indicates which types of probes are associated with which types of encoded carrier, and wherein more than one type of probe is associated with one type of encoded carrier;
e) contacting the set of encoded probes with said amplicons under conditions permitting the formation of a probe elongation product, following annealing of encoded probes to amplicons, and wherein probes are capable of annealing to an amplicon such that an interrogation site within a probe is in alignment with a designated polymorphic site in said amplicon; and
f) detecting probe elongation products.

71-106. (canceled)

107. The method of claim 70 wherein the interrogation site is at the 3′ terminus of the probe.

108. (canceled)

109. The method of claim 70 wherein the encoded probe set includes a subset of four different types of probes each, with a different nucleotide which aligns with a designated polymorphic site.

110. The method of claim 70 wherein said target is an mRNA, cDNA or a double-stranded polynucleotide including DNA.

111. The method of claim 70 wherein encoding of probes is by associating probes with different sequences to carriers, including beads, having different optical signatures.

112. The method of claim 111 wherein the encoding is with color.

113. The method of claim 70 wherein the elongation of the probes comprises adding one or more types of deoxyribonucleotide triphosphates or di-deoxyribonucleotide triphosphates for elongating the set of probes.

114. The method of claim 70 wherein only one type of deoxyribonucleotide triphosphates or di-deoxyribonucleotide triphosphate is involved in elongating the set of probes.

115. The method of claim 113 wherein a fraction of at least one type of deoxyribonucleotide triphosphate or di-deoxyribonucleotide triphosphate is labeled so as to generate an optically detectable signature associated with the elongation product following its incorporation into the probe.

116. The method of claim 113 wherein all types of deoxyribonucleotide triphosphate or di-deoxyribonucleotide triphosphate are labeled so as to generate an optically detectable signature associated with the elongation product following its incorporation into the probe.

117. The method of claim 116 wherein a polymerase is included for mediating the elongation of the probes.

118. The method of claim 117 wherein the polymerase lacks 3′->5′ exonuclease activity.

119. The method of claim 70 wherein one of the complementary strands of each amplicon pair is selectively removed by digesting it with an enzyme.

120. The method of claims 119 wherein an amplicon is preselected for digestion by phosphorylating the primer incorporated in it.

121. The method of claim 70 wherein the 3 base segment at the 3′ terminus of a probe is perfectly complementary to the subsequence including the designated polymorphism of the complementary amplicon strand.

122. The method of claim 70 wherein the excess of the amplicon sense strand is produced by digestion of said complementary amplicon antisense strand.

123. The method of claim 70 wherein the excess of the amplicon anisense strand is produced by digestion of said complementary amplicon sense strand.

124. A method for differentiating alleles which are differentiated by different nucleotides at a variable site of one or more nucleic acid sequences, comprising:
providing, for each designated variable site of a nucleic acid sequence, pairs of labeled primers, with members of a pair being differently labeled from each other and from other members of other pairs, one member having a subsequence complementary to a first subsequence which is identical to the 5′ terminal subsequence of one allele, and the other member of the pair being identical to said one member but for the nucleotide at its 3′ terminus, wherein, following annealing of said complementary subsequences on a primer with their respective complementary subsequences, said primers are capable, under appropriate conditions, of being extended to form elongation products by addition of dNTPs to their respective 3′ ends and wherein said labels are detectable in the elongation products;
providing, for each anticipated elongation product, an oligonucleotide probe optically detectably-labeled having a subsequence complementary to a subsequence of a particular elongation-product;
providing conditions for generation of elongation products;
combining elongation products with oligonucleotide probes under conditions permitting annealing of elongation products and oligonucleotide probes having complementary subsequences;
detecting elongation products by detecting the presence of the optically detectable labels; and
identifying different elongation products by detecting and identifying the labels on the detected elongation products.

125. The method of claim 124 wherein the optically detectable label is a fluorescent molecule.

126. The method of claim 124 wherein the subsequence of particular primers which is to the 5′ terminal subsequence of an allele is identical to said 5′ terminal subsequence on either the sense or the antisense strand, said particular primers selected so as to maximize the degree of complementary between said primer subsequence and said 5′ terminal subsequence.

127. The method of claim 124 wherein the oligonucleotide probe subsequence has a nucleotide aligned with the nucleotide on the elongation product which is complementary to the variable site.

128. The method of claim 124 wherein an elongation probe, on annealing, is aligned such that its 3′-end aligns with a base immediately adjacent to the base complementary to the variable site.

129. The method of claim 124 wherein extension of the primer to form an elongation product is catalyzed by a DNA polymerase or by a reverse transcriptase

130. The method of claim 124 wherein extension of the primer to form an elongation product is with a mixture of both dNTPs and ddNTPs

131. The method of claim 124 wherein the variable sites comprise single nucleotide polymorphism, insertions and deletions.


SEQUENCE LISTING (208 sequences)

SEQ ID NO: 1 - DNA, 20 bp, Homo sapiens
ttcttgtggcagcttaagtt

SEQ ID NO: 2 - DNA, 20 bp, Homo sapiens
ttcttggagtactctacgtc

SEQ ID NO: 3 - DNA, 20 bp, Homo sapiens
ttcttggagcaggttaaaca

SEQ ID NO: 4 - DNA, 20 bp, Homo sapiens
ttcttggagcaggttaaacc

SEQ ID NO: 5 - DNA, 20 bp, Homo sapiens
ttcctgtggcagggtaagta

SEQ ID NO: 6 - DNA, 20 bp, Homo sapiens
ttcctgtggcagggtaaata

SEQ ID NO: 7 - DNA, 20 bp, Homo sapiens
ttcttggagtactctacggg

SEQ ID NO: 8 - DNA, 20 bp, Homo sapiens
ttcttggagtactctagggg

SEQ ID NO: 9 - DNA, 20 bp, Homo sapiens
ttcttggagtactctacggc

SEQ ID NO: 10 - DNA, 20 bp, Homo sapiens
ttcttggagtactctatggg

SEQ ID NO: 11 - DNA, 20 bp, Homo sapiens
ttcttgaagcaggataagtt

SEQ ID NO: 12 - DNA, 20 bp, Homo sapiens
ttcttggaggaggttaagtt

SEQ ID NO: 13 - DNA, 20 bp, Homo sapiens
ttcttggagcaggctacaca

SEQ ID NO: 14 - DNA, 20 bp, Homo sapiens
ttcttggagttccttaagtc

SEQ ID NO: 15 - DNA, 20 bp, Homo sapiens
ttcctgtggcagcctaagag

SEQ ID NO: 16 - DNA, 20 bp, Homo sapiens
ttcttggagctgcgtaagtc

SEQ ID NO: 17 - DNA, 20 bp, Homo sapiens
ttcttggagctgtgtaagtc

SEQ ID NO: 18 - DNA, 20 bp, Homo sapiens
ttctcggagctgcgtaagtc

SEQ ID NO: 19 - DNA, 20 bp, Homo sapiens
ttcttggagctgcttaagtc

SEQ ID NO: 20 - DNA, 20 bp, Homo sapiens
ttcttgcagctgcttaagtc

SEQ ID NO: 21 - DNA, 20 bp, Homo sapiens
ttcttggagcaggctaagtg

SEQ ID NO: 22 - DNA, 20 bp, Homo sapiens
ttcttgcagcaggataagta

SEQ ID NO: 23 - DNA, 20 bp, Homo sapiens
ttcttggagtactctacgtc

SEQ ID NO: 24 - DNA, 20 bp, Homo sapiens
ttcttggagtgccttaagtc

SEQ ID NO: 25 - DNA, 20 bp, Homo sapiens
ttcttggagctgcgtaagtc

SEQ ID NO: 26 - DNA, 20 bp, Homo sapiens
ttcttggagctgtgtaagtc

SEQ ID NO: 27 - DNA, 20 bp, Homo sapiens
ttctcggagctgcgtaagtc

SEQ ID NO: 28 - DNA, 20 bp, Homo sapiens
ttcttggagctgcttaagtc

SEQ ID NO: 29 - DNA, 20 bp, Homo sapiens
ttcttgcagctgcttaagtc

SEQ ID NO: 30 - DNA, 20 bp, Homo sapiens
ttcttgtggcagcttaagtt

SEQ ID NO: 31 - DNA, 20 bp, Homo sapiens
ttcttgaagcaggataagtt

SEQ ID NO: 32 - DNA, 20 bp, Homo sapiens
ttcttggaggaggttaagtt

SEQ ID NO: 33 - DNA, 20 bp, Homo sapiens
ttcttggagcaggttaaaca

SEQ ID NO: 34 - DNA, 20 bp, Homo sapiens
ttcttggagcaggctacaca

SEQ ID NO: 35 - DNA, 20 bp, Homo sapiens
ttcttggagcaggttaaacc

SEQ ID NO: 36 - DNA, 20 bp, Homo sapiens
ttcctgtggcagggtaagta

SEQ ID NO: 37 - DNA, 20 bp, Homo sapiens
ttcttgcagcaggataagta

SEQ ID NO: 38 - DNA, 20 bp, Homo sapiens
ttcctgtggcagggtaaata

SEQ ID NO: 39 - DNA, 20 bp, Homo sapiens
ttcttggagtactctacggg

SEQ ID NO: 40 - DNA, 20 bp, Homo sapiens
ttcttggagtactctagggg

SEQ ID NO: 41 - DNA, 20 bp, Homo sapiens
ttcttggagtactctatggg

SEQ ID NO: 42 - DNA, 20 bp, Homo sapiens
ttcttggagtactctacggc

SEQ ID NO: 43 - DNA, 20 bp, Homo sapiens
ttcctgtggcagcctaagag

SEQ ID NO: 44 - DNA, 20 bp, Homo sapiens
ttcttggagcaggctaagtg

SEQ ID NO: 45 - DNA, 20 bp, Homo sapiens
ttcttggagctgcttaagtc

SEQ ID NO: 46 - DNA, 20 bp, Homo sapiens
ttcttggagttccttaagtc

SEQ ID NO: 47 - DNA, 20 bp, Homo sapiens
ttcttggagctgcgtaagtc

SEQ ID NO: 48 - DNA, 20 bp, Homo sapiens
ttcttggagctgtgtaagtc

SEQ ID NO: 49 - DNA, 20 bp, Homo sapiens
ttctcggagctgcgtaagtc

SEQ ID NO: 50 - DNA, 20 bp, Homo sapiens
ttcttgcagctgcttaagtc

SEQ ID NO: 51 - DNA, 17 bp, Homo sapiens
gacatcctggaagacga

SEQ ID NO: 52 - DNA, 17 bp, Homo sapiens
gacttcctggaagacga

SEQ ID NO: 53 - DNA, 17 bp, Homo sapiens
gacctcctggaagacga

SEQ ID NO: 54 - DNA, 12 bp, Homo sapiens
agccagaaggac

SEQ ID NO: 55 - DNA, 19 bp, Artificial Sequence
  Description of Artificial Sequence Synthetic probe
aaggacatcctggaagacg

SEQ ID NO: 56 - DNA, 19 bp, Artificial Sequence
  Description of Artificial Sequence Synthetic probe
aaggacatcctggaagaca

SEQ ID NO: 57 - DNA, 18 bp, Artificial Sequence
  Description of Artificial Sequence Synthetic probe
ataaccaggaggagttcc

SEQ ID NO: 58 - DNA, 18 bp, Artificial Sequence
  Description of Artificial Sequence Synthetic probe
ataaccaggaggagttcg

SEQ ID NO: 59 - DNA, 33 bp, Artificial Sequence
  Description of Artificial Sequence Synthetic probe
gtcgaagcgcaggaactcctcctggttatggaa

SEQ ID NO: 60 - DNA, 33 bp, Artificial Sequence
  Description of Artificial Sequence Synthetic probe
gtcgaagcgcacgaactcctcctggttatagaa

SEQ ID NO: 61 - DNA, 33 bp, Artificial Sequence
  Description of Artificial Sequence Synthetic probe
ggcccgctcgtcttccaggatgtccttctggct

SEQ ID NO: 62 - DNA, 33 bp, Artificial Sequence
  Description of Artificial Sequence Synthetic probe
ggcccgcttgtcttccaggatgtccttctggct

SEQ ID NO: 63 - DNA, 24 bp, Artificial Sequence
  Description of Artificial Sequence Synthetic primer
gatccttcgtgtccccacagcacg

SEQ ID NO: 64 - DNA, 22 bp, Artificial Sequence
  Description of Artificial Sequence Synthetic primer
gccgctgcactgtgaagctctc

SEQ ID NO: 65 - DNA, 18 bp, Artificial Sequence
  Description of Artificial Sequence Synthetic probe
acggagcgggtgcggttg

SEQ ID NO: 66 - DNA, 17 bp, Artificial Sequence
  Description of Artificial Sequence Synthetic probe
gctgtcgaagcgcacgg

SEQ ID NO: 67 - DNA, 19 bp, Artificial Sequence
  Description of Artificial Sequence Synthetic probe
cgctgtcgaagcgcacgtt

SEQ ID NO: 68 - DNA, 23 bp, Artificial Sequence
  Description of Artificial Sequence Synthetic probe
gttatggaagtatctgtccaggt

SEQ ID NO: 69 - DNA, 23 bp, Artificial Sequence
  Description of Artificial Sequence Synthetic probe
acgtttcttggagcaggttaaac

SEQ ID NO: 70 - DNA, 25 bp, Artificial Sequence
  Description of Artificial Sequence Synthetic probe
cgtttcctgtggcagggtaagtata

SEQ ID NO: 71 - DNA, 19 bp, Artificial Sequence
  Description of Artificial Sequence Synthetic probe
tcgctgtcgaagcgcacga

SEQ ID NO: 72 - DNA, 23 bp, Artificial Sequence
  Description of Artificial Sequence Synthetic probe
cgtttcttggagtactctacggg

SEQ ID NO: 73 - DNA, 20 bp, Artificial Sequence
  Description of Artificial Sequence Synthetic probe
tctgcagtaggtgtccacca

SEQ ID NO: 74 - DNA, 19 bp, Artificial Sequence
  Description of Artificial Sequence Synthetic probe
cacgtttcttggagctgcg

SEQ ID NO: 75 - DNA, 18 bp, Artificial Sequence
  Description of Artificial Sequence Synthetic probe
ggagtaccgggcggtgag

SEQ ID NO: 76 - DNA, 23 bp, Artificial Sequence
  Description of Artificial Sequence Synthetic probe
gtgtctgcagtaattgtccacct

SEQ ID NO: 77 - DNA, 19 bp, Artificial Sequence
  Description of Artificial Sequence Synthetic probe
ctgttccaggactcggcga

SEQ ID NO: 78 - DNA, 23 bp, Artificial Sequence
  Description of Artificial Sequence Synthetic probe
ctctccacaaccccgtagttgta

SEQ ID NO: 79 - DNA, 22 bp, Artificial Sequence
  Description of Artificial Sequence Synthetic probe
cgtttcctgtggcagcctaaga

SEQ ID NO: 80 - DNA, 15 bp, Artificial Sequence
  Description of Artificial Sequence Synthetic probe
caccgcggcccgcgc

SEQ ID NO: 81 - DNA, 18 bp, Artificial Sequence
  Description of Artificial Sequence Synthetic probe
gctgtcgaagcgcaagtc

SEQ ID NO: 82 - DNA, 18 bp, Artificial Sequence
  Description of Artificial Sequence Synthetic probe
gctgtcgaagcgcacgta

SEQ ID NO: 83 - DNA, 18 bp, Artificial Sequence
  Description of Artificial Sequence Synthetic probe
aaaaaaaaaaaaaaaaaa

SEQ ID NO: 84 - DNA, 17 bp, Artificial Sequence
  Description of Artificial Sequence Synthetic probe
gctgtcgaagcgcacgg

SEQ ID NO: 85 - DNA, 19 bp, Artificial Sequence
  Description of Artificial Sequence Synthetic probe
tcgctgtcgaagcgcacga

SEQ ID NO: 86 - DNA, 19 bp, Artificial Sequence
  Description of Artificial Sequence Synthetic probe
cgctgtcgaagcgcacgtt

SEQ ID NO: 87 - DNA, 18 bp, Artificial Sequence
  Description of Artificial Sequence Synthetic probe
gctgtcgaagcgcaagtc

SEQ ID NO: 88 - DNA, 18 bp, Artificial Sequence
  Description of Artificial Sequence Synthetic probe
gctgtcgaagcgcacgta

SEQ ID NO: 89 - DNA, 18 bp, Artificial Sequence
  Description of Artificial Sequence Synthetic probe
cactccacgcacgtgcca

SEQ ID NO: 90 - DNA, 18 bp, Artificial Sequence
  Description of Artificial Sequence Synthetic probe
gcgcaggtcctcgttcaa

SEQ ID NO: 91 - DNA, 18 bp, Artificial Sequence
  Description of Artificial Sequence Synthetic probe
ctccaggtaggctctcaa

SEQ ID NO: 92 - DNA, 18 bp, Artificial Sequence
  Description of Artificial Sequence Synthetic probe
ctccaggtaggctctctg

SEQ ID NO: 93 - DNA, 16 bp, Artificial Sequence
  Description of Artificial Sequence Synthetic probe
gcccgtccacgcaccg

SEQ ID NO: 94 - DNA, 17 bp, Artificial Sequence
  Description of Artificial Sequence Synthetic probe
ggtatctgcggagcccg

SEQ ID NO: 95 - DNA, 19 bp, Artificial Sequence
  Description of Artificial Sequence Synthetic probe
catccaggtaggctctcaa

SEQ ID NO: 96 - DNA, 18 bp, Artificial Sequence
  Description of Artificial Sequence Synthetic probe
gccggagtattgggacga

SEQ ID NO: 97 - DNA, 18 bp, Artificial Sequence
  Description of Artificial Sequence Synthetic probe
tggatagagcaggagggt

SEQ ID NO: 98 - DNA, 19 bp, Artificial Sequence
  Description of Artificial Sequence Synthetic probe
gaccaggagacacggaata

SEQ ID NO: 99 - DNA, 17 bp, Artificial Sequence
  Description of Artificial Sequence Synthetic probe
ccgcgcgctccagcgtg

SEQ ID NO: 100 - DNA, 20 bp, Artificial Sequence
  Description of Artificial Sequence Synthetic probe
ccactccatgaggtatttcc

SEQ ID NO: 101 - DNA, 18 bp, Artificial Sequence
  Description of Artificial Sequence Synthetic probe
ctccaacttgcgctggga

SEQ ID NO: 102 - DNA, 18 bp, Artificial Sequence
  Description of Artificial Sequence Synthetic probe
cgccacgagtccgaggaa

SEQ ID NO: 103 - DNA, 18 bp, Artificial Sequence
  Description of Artificial Sequence Synthetic probe
gtcgtaggcgtcctggtc

SEQ ID NO: 104 - DNA, 18 bp, Artificial Sequence
  Description of Artificial Sequence Synthetic probe
taccagcgcgctccagct

SEQ ID NO: 105 - DNA, 17 bp, Artificial Sequence
  Description of Artificial Sequence Synthetic probe
agcaggaggggccggaa

SEQ ID NO: 106 - DNA, 19 bp, Artificial Sequence
  Description of Artificial Sequence Synthetic probe
cgtcgcagccatacatcca

SEQ ID NO: 107 - DNA, 18 bp, Artificial Sequence
  Description of Artificial Sequence Synthetic probe
gcgccgtggatagagcaa

SEQ ID NO: 108 - DNA, 17 bp, Artificial Sequence
  Description of Artificial Sequence Synthetic probe
gccgcgagtccgaggac

SEQ ID NO: 109 - DNA, 20 bp, Artificial Sequence
  Description of Artificial Sequence Synthetic probe
gaccggaacacacagatctt

SEQ ID NO: 110 - DNA, 21 bp, Homo sapiens
cccctaaatataaaaagattc

SEQ ID NO: 111 - DNA, 21 bp, Homo sapiens
cccctaaatataaaaagattt

SEQ ID NO: 112 - DNA, 19 bp, Homo sapiens
attctcatctccattccaa

SEQ ID NO: 113 - DNA, 19 bp, Homo sapiens
attctcatctccattccag

SEQ ID NO: 114 - DNA, 20 bp, Homo sapiens
tgtgtgcaaggaagtattac

SEQ ID NO: 115 - DNA, 20 bp, Homo sapiens
tgtgtgcaaggaagtattaa

SEQ ID NO: 116 - DNA, 20 bp, Homo sapiens
tagataaatcgcgatagagc

SEQ ID NO: 117 - DNA, 20 bp, Homo sapiens
tagataaatcgcgatagagt

SEQ ID NO: 118 - DNA, 19 bp, Homo sapiens
taaatcaataggtacatac

SEQ ID NO: 119 - DNA, 19 bp, Homo sapiens
taaatcaataggtacataa

SEQ ID NO: 120 - DNA, 20 bp, Homo sapiens
atggtggtgaatattttccg

SEQ ID NO: 121 - DNA, 20 bp, Homo sapiens
atggtggtgaatattttcca

SEQ ID NO: 122 - DNA, 21 bp, Homo sapiens
attgccgagtgaccgccatgc

SEQ ID NO: 123 - DNA, 21 bp, Homo sapiens
attgccgagtgaccgccatgg

SEQ ID NO: 124 - DNA, 21 bp, Homo sapiens
cacagataaaaacaccacaaa

SEQ ID NO: 125 - DNA, 20 bp, Homo sapiens
cacagataaaaacaccacaa

SEQ ID NO: 126 - DNA, 19 bp, Homo sapiens
cacagataaaaacaccaca

SEQ ID NO: 127 - DNA, 20 bp, Homo sapiens
tccagtggatccagcaaccg

SEQ ID NO: 128 - DNA, 20 bp, Homo sapiens
tccagtggatccagcaacct

SEQ ID NO: 129 - DNA, 19 bp, Homo sapiens
cataggaaacaccaaagat

SEQ ID NO: 130 - DNA, 16 bp, Homo sapiens
cataggaaacaccaaa

SEQ ID NO: 131 - DNA, 16 bp, Homo sapiens
cataggaaacaccaat

SEQ ID NO: 132 - DNA, 20 bp, Homo sapiens
ctgcaaacttggagatgtcc

SEQ ID NO: 133 - DNA, 20 bp, Homo sapiens
ctgcaaacttggagatgtct

SEQ ID NO: 134 - DNA, 15 bp, Homo sapiens
ttcttgctcgttgac

SEQ ID NO: 135 - DNA, 15 bp, Homo sapiens
ttcttgctcgttgat

SEQ ID NO: 136 - DNA, 18 bp, Homo sapiens
taaagaaattcttgctcg

SEQ ID NO: 137 - DNA, 18 bp, Homo sapiens
taaagaaattcttgctca

SEQ ID NO: 138 - DNA, 21 bp, Homo sapiens
accaataattagttattcacc

SEQ ID NO: 139 - DNA, 21 bp, Homo sapiens
accaataattagttattcacg

SEQ ID NO: 140 - DNA, 19 bp, Homo sapiens
gtgtgattccaccttctcc

SEQ ID NO: 141 - DNA, 19 bp, Homo sapiens
gtgtgattccaccttctca

SEQ ID NO: 142 - DNA, 19 bp, Homo sapiens
aggtattcaaagaacatac

SEQ ID NO: 143 - DNA, 19 bp, Homo sapiens
aggtattcaaagaacatat

SEQ ID NO: 144 - DNA, 19 bp, Homo sapiens
tgtctgtttaaaagattgt

SEQ ID NO: 145 - DNA, 19 bp, Homo sapiens
tgtctgtttaaaagattgc

SEQ ID NO: 146 - DNA, 18 bp, Homo sapiens
caataggacatggaatac

SEQ ID NO: 147 - DNA, 19 bp, Homo sapiens
caataggacatggaatact

SEQ ID NO: 148 - DNA, 16 bp, Homo sapiens
acttatttttacatac

SEQ ID NO: 149 - DNA, 16 bp, Homo sapiens
acttatttttacatat

SEQ ID NO: 150 - DNA, 21 bp, Homo sapiens
acttaccaagctatccacatc

SEQ ID NO: 151 - DNA, 21 bp, Homo sapiens
acttaccaagctatccacatg

SEQ ID NO: 152 - DNA, 21 bp, Homo sapiens
cctttcagggtgtcttactcg

SEQ ID NO: 153 - DNA, 21 bp, Homo sapiens
cctttcagggtgtcttactca

SEQ ID NO: 154 - DNA, 18 bp, Homo sapiens
aatgaacttaaagactcg

SEQ ID NO: 155 - DNA, 18 bp, Homo sapiens
aatgaacttaaagactca

SEQ ID NO: 156 - DNA, 20 bp, Homo sapiens
gtatggtttggttgacttgg

SEQ ID NO: 157 - DNA, 21 bp, Homo sapiens
gtatggtttggttgacttgta

SEQ ID NO: 158 - DNA, 22 bp, Homo sapiens
gtatggtttggttgacttggta

SEQ ID NO: 159 - DNA, 21 bp, Homo sapiens
gtatggtttggttgacttgta

SEQ ID NO: 160 - DNA, 18 bp, Homo sapiens
actccaaaggctttcctc

SEQ ID NO: 161 - DNA, 17 bp, Homo sapiens
ctccaaaggctttcctt

SEQ ID NO: 162 - DNA, 18 bp, Homo sapiens
tgttcatagggatccaag

SEQ ID NO: 163 - DNA, 18 bp, Homo sapiens
tgttcatagggatccaac

SEQ ID NO: 164 - DNA, 16 bp, Homo sapiens
aggactccatgcccag

SEQ ID NO: 165 - DNA, 831 bp, Homo sapiens
cactgtagctgtactaccttccatctcctcaacctattccaactatctgaatcatgtgcccttctctgtgaacctctatcataatacttgtcacactgtattgtaattgtctcttttactttcccttgtatcttttgtgcatagcagagtacctgaaacaggaagtattttaaatattttgaatcaaatgagttaatagaatctttacaaataagaatatacacttctgcttaggatgataattggaggcaagtgaatcctgagcgtgatttgataatgacctaataatgatgggttttatttccagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggtaagaaactatgtgaaaactttttgattatgcatatgaacccttcacactacccaaattatatatttggctccatattcaatcggttagtctacatatatttatgtttcctctatgggtaagctactgtgaatggatcaattaataaaacacatgacctatgctttaagaagcttgcaaacacatgaaataaatgcaatttattttttaaataatgggttcatttgatcacaataaatgcattttatgaaatggtgagaattttgttcactcattagtgagacaaacgtctcaatggttatttatatggcatgcatatagtgatatgtggt

SEQ ID NO: 166 - DNA, 19 bp, Homo sapiens
gcatggcggtcactcggca

SEQ ID NO: 167 - DNA, 19 bp, Artificial Sequence
  Description of Artificial Sequence Synthetic probe
tycryagtgaccyccytgn

SEQ ID NO: 168 - DNA, 18 bp, Artificial Sequence
  Description of Artificial Sequence Synthetic probe
cgggagtgaccgccatgc

SEQ ID NO: 169 - DNA, 18 bp, Artificial Sequence
  Description of Artificial Sequence Synthetic probe
cgtaccgccagtgagngn

SEQ ID NO: 170 - DNA, 18 bp, Artificial Sequence
  Description of Artificial Sequence Synthetic probe
cgtaccgccagtgacngn

SEQ ID NO: 171 - DNA, 18 bp, Artificial Sequence
  Description of Artificial Sequence Synthetic probe
cgtacccccagtgagngn

SEQ ID NO: 172 - DNA, 18 bp, Artificial Sequence
  Description of Artificial Sequence Synthetic probe
cgtacccccagtgacngn

SEQ ID NO: 173 - DNA, 18 bp, Artificial Sequence
  Description of Artificial Sequence Synthetic probe
cgttccgccagtgagngn

SEQ ID NO: 174 - DNA, 18 bp, Artificial Sequence
  Description of Artificial Sequence Synthetic probe
cgttccgccagtgacngn

SEQ ID NO: 175 - DNA, 18 bp, Artificial Sequence
  Description of Artificial Sequence Synthetic probe
cgttcccccagtgagngn

SEQ ID NO: 176 - DNA, 18 bp, Artificial Sequence
  Description of Artificial Sequence Synthetic probe
cgttcccccagtgacngn

SEQ ID NO: 177 - DNA, 18 bp, Artificial Sequence
  Description of Artificial Sequence Synthetic probe
ggtaccgccagtgagngn

SEQ ID NO: 178 - DNA, 18 bp, Artificial Sequence
  Description of Artificial Sequence Synthetic probe
ggtaccgccagtgacngn

SEQ ID NO: 179 - DNA, 18 bp, Artificial Sequence
  Description of Artificial Sequence Synthetic probe
ggtacccccagtgagngn

SEQ ID NO: 180 - DNA, 18 bp, Artificial Sequence
  Description of Artificial Sequence Synthetic probe
ggtacccccagtgacngn

SEQ ID NO: 181 - DNA, 18 bp, Artificial Sequence
  Description of Artificial Sequence Synthetic probe
ggttccgccagtgagngn

SEQ ID NO: 182 - DNA, 18 bp, Artificial Sequence
  Description of Artificial Sequence Synthetic probe
ggttccgccagtgacngn

SEQ ID NO: 183 - DNA, 18 bp, Artificial Sequence
  Description of Artificial Sequence Synthetic probe
ggttcccccagtgagngn

SEQ ID NO: 184 - DNA, 18 bp, Artificial Sequence
  Description of Artificial Sequence Synthetic probe
ggttcccccagtgacngn

SEQ ID NO: 185 - DNA, 18 bp, Artificial Sequence
  Description of Artificial Sequence Synthetic probe
agtaccgccagtgagngn

SEQ ID NO: 186 - DNA, 18 bp, Artificial Sequence
  Description of Artificial Sequence Synthetic probe
agtaccgccagtgacngn

SEQ ID NO: 187 - DNA, 18 bp, Artificial Sequence
  Description of Artificial Sequence Synthetic probe
agtacccccagtgagngn

SEQ ID NO: 188 - DNA, 18 bp, Artificial Sequence
  Description of Artificial Sequence Synthetic probe
agtacccccagtgacngn

SEQ ID NO: 189 - DNA, 18 bp, Artificial Sequence
  Description of Artificial Sequence Synthetic probe
agttccgccagtgagngn

SEQ ID NO: 190 - DNA, 18 bp, Artificial Sequence
  Description of Artificial Sequence Synthetic probe
agttccgccagtgacngn

SEQ ID NO: 191 - DNA, 18 bp, Artificial Sequence
  Description of Artificial Sequence Synthetic probe
agttcccccagtgagngn

SEQ ID NO: 192 - DNA, 18 bp, Artificial Sequence
  Description of Artificial Sequence Synthetic probe
agttcccccagtgacngn

SEQ ID NO: 193 - DNA, 18 bp, Artificial Sequence
  Description of Artificial Sequence Synthetic probe
tgtaccgccagtgagngn

SEQ ID NO: 194 - DNA, 18 bp, Artificial Sequence
  Description of Artificial Sequence Synthetic probe
tgtaccgccagtgacngn

SEQ ID NO: 195 - DNA, 18 bp, Artificial Sequence
  Description of Artificial Sequence Synthetic probe
tgtacccccagtgagngn

SEQ ID NO: 196 - DNA, 18 bp, Artificial Sequence
  Description of Artificial Sequence Synthetic probe
tgtacccccagtgacngn

SEQ ID NO: 197 - DNA, 18 bp, Artificial Sequence
  Description of Artificial Sequence Synthetic probe
tgttccgccagtgagngn

SEQ ID NO: 198 - DNA, 18 bp, Artificial Sequence
  Description of Artificial Sequence Synthetic probe
tgttccgccagtgacngn

SEQ ID NO: 199 - DNA, 18 bp, Artificial Sequence
  Description of Artificial Sequence Synthetic probe
tgttcccccagtgagngn

SEQ ID NO: 200 - DNA, 18 bp, Artificial Sequence
  Description of Artificial Sequence Synthetic probe
tgttcccccagtgacngn

SEQ ID NO: 201 - DNA, 36 bp, Artificial Sequence
  Description of Artificial Sequence Synthetic oligonucleotide
ggaacactctatcgcgatttatctaggcataggctt

SEQ ID NO: 202 - DNA, 19 bp, Artificial Sequence
  Description of Artificial Sequence Synthetic oligonucleotide
tgagatagcgctaaataga

SEQ ID NO: 203 - DNA, 36 bp, Artificial Sequence
  Description of Artificial Sequence Synthetic oligonucleotide
ggaaccctctatcgcgatttatctaggcataggctt

SEQ ID NO: 204 - DNA, 19 bp, Artificial Sequence
  Description of Artificial Sequence Synthetic oligonucleotide
ggagatagcgctaaataga

SEQ ID NO: 205 - DNA, 36 bp, Artificial Sequence
  Description of Artificial Sequence Synthetic oligonucleotide
ggaacgctctatcgcgatttatctaggcataggctt

SEQ ID NO: 206 - DNA, 19 bp, Artificial Sequence
  Description of Artificial Sequence Synthetic oligonucleotide
cgagatagcgctaaataga

SEQ ID NO: 207 - DNA, 36 bp, Artificial Sequence
  Description of Artificial Sequence Synthetic oligonucleotide
ggaactctctatcgcgatttatctaggcataggctt

SEQ ID NO: 208 - DNA, 19 bp, Artificial Sequence
  Description of Artificial Sequence Synthetic oligonucleotide
agagatagcgctaaataga